(19) United States
(12) Patent Application Publication
(10) Pub. No.: US 2025/0186410 A1
(43) Pub. Date: Jun. 12, 2025

(54) DUAL INHIBITORS OF TRYPTOPHAN DIOXYGENASES (IDO1 AND TDO) AND THEIR USE IN THERAPY

(71) Applicant: ANTIDO THERAPEUTICS INTERNATIONAL SÀRL, Basel (CH)

(72) Inventors: Michael John BICKERDIKE, Auckland (NZ); Medhi WANGPAICHITR, Miami, FL (US)

(21) Appl. No.: 18/843,511

(22) PCT Filed: Mar. 4, 2023
(86) PCT No.: PCT/IB2023/052028

§ 371 Date: Sep. 3, 2024

Related U.S. Application Data
(60) Provisional application No. 63/316,740, filed on Mar. 4, 2022.

(51) Int. Cl. A61K 31/437; A61K 33/243; A61K 39/00; A61K 45/06; A61P 35/00; C07K 16/28

(52) U.S. Cl. CPC A61K 31/437; A61K 33/243; A61K 45/06; A61P 35/00; C07K 16/2818; A61K 2039/505

(57) ABSTRACT

The present invention generally relates to the use of a dual inhibitor of indoleamine 2,3-dioxygenase 1 (IDO1) and tryptophan-2,3-dioxygenase (TDO), either alone or in combination with other agents, and pharmaceutical compositions comprising the same in the treatment or prevention of refractory cancer and in the manufacture of a medicament for treating or preventing refractory cancer.

### CROSS-REFERENCE TO RELATED APPLICATIONS

This application is a U.S. National Entry of International Patent Application No. PCT/IB2023/052028, filed Mar. 4, 2023, which claims the benefit of priority of U.S. Provisional Patent Application No. 63/316,740, filed Mar. 4, 2022.

### TECHNICAL FIELD

The present invention generally relates to the use of a dual inhibitor of indoleamine 2,3-dioxygenase 1 (IDO1) and tryptophan-2,3-dioxygenase (TDO), either alone or in combination with other agents, and pharmaceutical compositions comprising the same in the treatment or prevention of refractory cancer and in the manufacture of a medicament for treating or preventing refractory cancer.

### BACKGROUND TO THE INVENTION

The essential amino acid tryptophan is principally broken down by two closely related enzymes, indoleamine 2,3-dioxygenase 1 (IDO1) and tryptophan-2,3-dioxygenase (TDO). Both enzymes catalyse the rate-limiting conversion of tryptophan to N-formylkynurenine, with subsequent degradation to kynurenine, in the kynurenine pathway.

Indoleamine 2,3-dioxygenase 1 (IDO1) has a low level of natural expression throughout the body, but is expressed in a broad range of cancers to suppress the immune system (Uyttenhove et al., J. Nat. Med. 2003, 9, 1269). High IDO1 expression in clinical tumours has been shown to correlate with poor patient prognosis in a wide range of cancers including lung, colorectal, breast, melanoma and gynaecologic cancers. Silencing of the IDO1 gene in a murine melanoma cell line resulted in reduced capacity of the cells to form tumours when implanted into mice (Zheng et al., J. Immunol. 2006, 177, 5639), supporting IDO1 inhibition as a method of cancer intervention. A number of groups have pursued the development of small molecule inhibitors of IDO1 as an approach for restoring tumour immunity in cancer patients.

Unlike IDO1, tryptophan-2,3-dioxygenase (TDO) has a high level of natural expression, predominantly in the liver. However, like IDO1, it also has been shown to have raised levels in tumours and offer a further target for anticancer agents (Pilotte et al PNAS 2012, 109, 2497).

Common treatments for cancer such as radiation, chemotherapy and immunotherapy are often limited in efficacy due to the development of resistance, and there remains a number of cancers with extremely poor prognosis. There is an ongoing need for methods of treating and preventing the development of such refractory cancers.

It is an object of the present invention to go some way to meeting this need, and/or at least to provide the public with a useful choice.

Other objects of the invention may become apparent from the following description which is given by way of example only.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### SUMMARY OF THE INVENTION

In a first particular aspect, the present invention broadly consists of a method of treating refractory cancer or preventing the development of refractory cancer in a subject, the method comprising administering to the subject in need thereof a therapeutically effective amount of a dual inhibitor of indoleamine-2,3-dioxygenase (IDO1) and tryptophan-2,3-dioxygenase (TDO).

In a second particular aspect, the present invention broadly consists of the use of a dual inhibitor of IDO1 and TDO in the manufacture of a medicament for treating refractory cancer or preventing the development of refractory cancer in a subject.

In a third particular aspect, the present invention broadly consists of a dual inhibitor of IDO1 and TDO for use in the treatment of refractory cancer or preventing the development of refractory cancer in a subject.

In a fourth particular aspect, the present invention broadly consists of a method of treating a refractory cancer cell or preventing the development of a refractory cancer cell, the method comprising administering a dual inhibitor of IDO1 and TDO to said cancer cell.

In a fifth particular aspect, the present invention broadly consists of the use of a dual inhibitor of IDO1 and TDO in the manufacture of a medicament for treating a refractory cancer cell or preventing the development of a refractory cancer cell.

In a sixth particular aspect, the present invention broadly consists in a dual inhibitor comprising dual inhibitor of IDO1 and TDO for use in treating a refractory cancer cell or preventing the development of a refractory cancer cell.

In a seventh particular aspect, the present invention provides a kit comprising a dual inhibitor of IDO1 and TDO; and optionally one or more additional therapeutic agents; and instructions for using the dual inhibitor in the method of the first or fourth aspect.

The following embodiments and preferences may relate alone or in any combination of any two or more to any of the above aspects.

In various embodiments, the method, use or dual inhibitor is for treating refractory cancer in a subject in need thereof. In various embodiments, the method, use or dual inhibitor is for preventing the development of refractory cancer in a subject in need thereof.

In various embodiments, the method, use or dual inhibitor is for treating a refractory cancer cell. In various embodiments, the method, use or dual inhibitor is for preventing the development of a refractory cancer cell.

In various embodiments, the refractory cancer cell is in vitro or in vivo.

In various embodiments, the refractory cancer or cancer cell is refractory to an anticancer agent.

In various embodiments, the refractory cancer or cancer cell is refractory to a cancer therapy. For example, in various embodiments, the cancer therapy comprises a step of administering one or more anticancer agent and/or a radiotherapy.

In various embodiments, the subject or refractory cancer cell has previously been administered an anticancer agent, and the refractory cancer or cancer cell is refractory to said anticancer agent.

In various embodiments, the refractory cancer or cancer cell is refractory to a cancer therapy. In various embodiments, the subject or refractory cancer cell has previously been administered a cancer therapy and the refractory cancer or cancer cell is refractory to said cancer therapy. In various embodiments, the cancer therapy comprises a step of administering one or more anticancer agent. In various embodiments, the cancer therapy is radiotherapy.

In various embodiments, the anticancer agent is selected from: a chemotherapeutic agent or a radiotherapeutic agent.

In various embodiments, the chemotherapeutic agent is a platinum-based chemotherapeutic agent. In various embodiments, the platinum-based chemotherapeutic agent is selected from: carboplatin, cisplatin, lobaplatin, oxaliplatin, picoplatin, nedaplatin, phenanthriplatin and satraplatin. In various embodiment, the platinum-based chemotherapeutic agent is cisplatin, carboplatin or oxaliplatin. In various embodiment, the platinum-based chemotherapeutic agent is cisplatin or oxaliplatin.

In various embodiment, the anticancer agent induces ROS accumulation within cancer cells.

In various embodiment, the anticancer agent induces ROS accumulation within cancer cells, and the refractory cancer or a refractory cancer cell is resistant to said ROS accumulation within the cancer cells. For example, in various embodiment, the refractory cancer or refractory cancer cell is resistant to ROS-induced cell death. For example, in various embodiments, the refractory cancer or refractory cancer cell is resistant to ROS dependent programmed cell death (PCD) in cancer cells.

In various embodiments, the refractory cancer or refractory cancer cell is colorectal cancer, breast cancer, melanoma, reproductive organ cancer, respiratory tract cancer, brain cancer, digestive tract cancer, urinary tract cancer, eye cancer, liver cancer, skin cancer, head and neck cancer, thyroid cancer, parathyroid cancer and/or their distant metastases.

In various embodiments, the refractory cancer or refractory cancer cell is lymphoma, sarcoma or leukemia.

In various embodiments, the refractory cancer or refractory cancer cell is selected from the group consisting of: refractory breast cancer selected from: invasive ductal carcinoma, invasive lobular carcinoma, ductal carcinoma in situ, and lobular carcinoma in situ; refractory cancer of the respiratory tract selected from: small-cell and non-small-cell lung carcinoma, bronchial adenoma and pleuropulmonary blastoma. In various embodiments, the refractory cancer is refractory brain cancer selected from: glioblastoma, brain stem and hypophthalmic glioma, cerebellar and cerebral astrocytoma, medulloblastoma, ependymoma, neuroectodermal and pineal tumor; refractory tumor of the male reproductive organs selected from: prostate and testicular cancer; refractory tumor of the female reproductive organs selected from: endometrial, cervical, ovarian, ovarian adenocarcinoma, vaginal, and vulvar cancer and sarcoma of the uterus; refractory tumor of the digestive tract selected from: anal, colon, colorectal, esophageal, gallblader, gastric, pancreatic, rectal, small-intestine, and salivary gland cancer; refractory tumors of the urinary tract selected from: bladder, penile, kidney, renal pelvis, ureter, and urethral cancer; refractory eye cancer selected from: intraocular melanoma and retinoblastoma; refractory liver cancer selected from: hepatocellular carcinoma (liver cell carcinomas with or without fibrolamellar variant), cholangiocarcinoma (intrahepatic bile duct carcinoma), and mixed hepatocellular cholangiocarcinoma; refractory skin cancer selected from: squamous cell carcinoma, Kaposi's sarcoma, malignant melanoma, Merkel cell skin cancer, and non-melanoma skin cancer; refractory head-and-neck cancer selected from laryngeal/hypopharyngeal/nasopharyngeal/oropharyngeal cancer, and lip and oral cavity cancer; refractory lymphoma selected from: AIDS-related lymphoma, non-Hodgkin's lymphoma, cutaneous T-cell lymphoma, Hodgkin's disease, and lymphoma of the central nervous system; refractory sarcoma selected from: sarcoma of the soft tissue, osteosarcoma, malignant fibrous histiocytoma, lymphosarcoma, and rhabdomyosarcoma; and refractory leukemia selected from: acute myeloid leukemia, acute lymphoblastic leukemia, chronic lymphocytic leukemia, chronic myelogenous leukemia, and hairy cell leukemia, and/or their distant metastases.

In various embodiments, the refractory cancer or refractory cancer cell is selected from lung cancer, pancreatic cancer, breast cancer and ovarian cancer.

In various embodiments, the anticancer agent is a platinum-based chemotherapeutic agent and the refractory cancer is selected from: lung cancer, pancreatic cancer, breast cancer and ovarian cancer. For example, in various embodiments, the anticancer agent is selected from cisplatin, carboplatin or oxaliplatin.

The refractory cancer may be a solid tumor or a liquid tumor. In various embodiments, the refractory cancer is a solid tumor.

In various embodiments, the dual inhibitors of IDO1 and TDO that may be used in any of the methods or uses (including in any use in the manufacture of a medicament as described herein and any inhibitor of IDO1 and TDO for use as described herein) as described herein, are described in the numbered paragraphs (1) to (49) below.
(1) A compound of Formula I or a pharmaceutically acceptable salt thereof, wherein the compound is a dual inhibitor of IDO1 and TDO, and wherein:

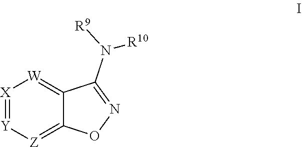

W is CR¹, N or N-oxide;
X is CR², N or N-oxide;
Y is CR³, N or N-oxide;
Z is CR⁴, N or N-oxide;
and where at least one of W, X, Y, and Z is N or N-oxide;
R¹, R², R³ and R⁴ are each independently selected from the following groups: H, halo, R, —OH, —OR, —OC(O)H, —OC(O)R, —OC(O)NH₂, —OC(O)NHR, —OC(O)NRR, —OP(O)(OH)₂, —OP(O)(OR)₂, —NO₂, —NH₂, —NHR, —NRR, —NHC(O)H, —NHC(O)R, —NRC(O)R, —NHC(O)NH₂, —NHC(O)NRR, —NRC(O)NHR, —SH, —SR, —S(O)H, —S(O)R, —SO₂R, —SO₂NH₂, —SO₂NHR, —SO₂NRR, —CF₃, —CHF₂, —CH₂F, —OCF₃, —OCHF₂, —CN, —C—CH, —C—CR, —CH═CHR, —CH═CRR, —CR═CHR, —CR═CRR, —CO₂H, —CO₂R, —CHO, —C(O)R, —C(O)NH₂, —C(O)NHR, —C(O)NRR, —CONHSO₂H, —CONHSO₂R, —CONRSO₂R, cyclic C₃-C₇ alkylamino, imidazolyl, C₁-C₆-alkylpiperazinyl, morpholinyl and thiomorpholinyl;
or R¹ and R² taken together, or R² and R³ taken together, or R³ and R⁴ taken together can form a saturated or a partially saturated or a fully unsaturated 5- or 6-membered ring of carbon atoms optionally including 1 to 3 heteroatoms selected from O, N and S, and the ring is optionally substituted independently with 1 to 4 substituents selected from R;
each R is independently selected from any of the groups defined in paragraphs (a) and (b) below:
(a) an optionally substituted C₁₋₆ alkyl group, an optionally substituted C₂₋₆ alkenyl group, an optionally substituted C₂₋₆ alkynyl group and an optionally substituted C₃₋₇ cyclic alkyl group; wherein the one or more optional substituents for each of said alkyl, alkenyl, alkynyl and cyclic alkyl groups are each independently selected from the following groups: halo, —OH, —OR⁵, —OC(O)R⁵, —OC(O)NH₂, —OC(O)NHR⁵, —OC(O)NR⁵R⁵, —OP(O)(OH)₂, —OP(O)(OR⁵)₂, —NO₂, —NH₂, —NHR⁵, —NR⁵R⁵, —N⁺(O⁻)R⁵R⁵, —NHC(O)H, —NHC(O)R⁵, —NR⁵C(O)R⁵, —NHC(O)NH₂, —NHC(O)NR⁵R⁵, —NR⁵C(O)NHR⁵, —SH, —SR⁵, —S(O)H, —S(O)R⁵, —SO₂R⁵, —SO₂NH₂, —SO₂NHR⁵, —SO₂NR⁵R⁵, —CF₃, —CHF₂, —CH₂F, —OCF₃, —OCHF₂, —CN, —CO₂H, —CO₂R⁵, —CHO, —C(O)R⁵, —C(O)NH₂, —C(O)NHR⁵, —C(O)NR⁵R⁵, —CONHSO₂H, —C(O)NHSO₂R⁵, —C(O)NR⁵SO₂R⁵, cyclic C₃-C₇ alkylamino, imidazolyl, piperazinyl, morpholinyl, thiomorpholinyl, piperidinyl, azepanyl, pyrrolidinyl and azetidinyl; wherein each of the groups imidazolyl, piperazinyl, morpholinyl, thiomorpholinyl, piperidinyl, azepanyl, pyrrolidinyl and azetidinyl are optionally substituted by one or more of the following groups: C₁₋₆ alkyl, C₂₋₆ alkenyl, C₂₋₆ alkynyl, C₃₋₇ cyclic alkyl, halo, —OH, —OR⁷, —OC(O)R⁷, —OC(O)NH₂—OC(O)NHR⁷, —OC(O)NR⁷R⁷, —OP(O)(OH)₂, —OP(O)(OR⁷)₂, —NO₂, —NH₂, —NHR⁷, —NR⁷R⁷, —N⁺(O⁻) R⁷R⁷, —NHC(O)H, —NHC(O)R⁷, —NR⁷C(O)R⁷, —NHC(O)NH₂, —NHC(O)NR⁷R⁷, —NR⁷C(O)NHR⁷, —SH, —SR⁷, —S(O)H, —S(O)R⁷, —SO₂R⁷, —SO₂NH₂, —SO₂NHR⁷, —SO₂NR⁷R⁷, —CF₃, —CHF₂, —CH₂F, —OCF₃, —OCHF₂, —CN, —CO₂H, —CO₂R⁷, —CHO, —C(O)R⁷, —C(O)NH₂, —C(O)NHR⁷, —C(O)NR⁷R⁷, —CONHSO₂H, —C(O)NHSO₂R⁷, —C(O)NRSO₂R⁷, an optionally substituted aryl, and an optionally substituted heteroaryl group having up to 12 carbon atoms and having one or more heteroatoms in its ring system which are each independently selected from O, N and S; and wherein the one or more optional substituents for each of said aryl and heteroaryl groups are each independently selected from the following groups: C₁₋₆ alkyl, C₂₋₆ alkenyl, C₂₋₆ alkynyl or C₃₋₇ cyclic alkyl, halo, —OH, —OR⁸, —OC(O)R⁸, —OC(O)NH₂, —OC(O)NHR⁸, —OC(O)NR⁸R⁸, —OP(O)(OH)₂, —OP(O)(OR⁸)₂, —NO₂, —NH₂, —NHR⁸, —NR⁸R⁸, —N⁺(O)R⁸R⁸, —NHC(O)H, —NHC(O)R⁸, —NR⁸C(O)R⁸, —NHC(O)NH₂, —NHC(O)NR⁸R⁸, —NR⁸C(O)NHR⁸, —SH, —SR⁸, —S(O)H, —S(O)R⁸, —SO₂R⁸, —SO₂NH₂, —SO₂NHR⁸, —SO₂NR⁸R⁸, —CF₃, —CHF₂, —CH₂F, —OCF₃, —OCHF₂, —CN, —CO₂H, —CO₂R⁸, —CHO, —C(O)R⁸, —C(O)NH₂, —C(O)NHR⁸, —C(O)NR⁸R⁸, —CONHSO₂H, —C(O)NHSO₂R⁸, and —C(O)NR⁸SO₂R⁸; wherein each R⁵, R⁷ and R⁸ is independently selected from a C₁₋₆ alkyl group, a C₂₋₆ alkenyl group, a C₂₋₆ alkynyl group and a C₃₋₇ cyclic alkyl group; and
(b) an optionally substituted aryl, and an optionally substituted heteroaryl group having up to 12 carbon atoms and having one or more heteroatoms in its ring system which are each independently selected from 0, N and S; and wherein the one or more optional substituents are each independently selected from the same optional substituents as those defined in (a) above for R;
R⁹ and R¹⁰ are each independently selected from any of the groups defined in paragraphs (a) and (b) below:
(a) H, an optionally substituted C₁₋₆ alkyl group, an optionally substituted C₂₋₆ alkenyl group, an optionally substituted C₂₋₆ alkynyl group, and an optionally substituted C₃₋₇ cyclic alkyl group; wherein the one or more optional substituents for each of said alkyl, alkenyl, alkynyl and cyclic alkyl are each independently selected from the following groups: halo, —OH, —OR¹¹, —OC(O)R¹¹, —OC(O)NH₂, —OC(O)NHR¹¹, —OC(O)NR¹¹R¹¹, —OP(O)(OH)₂, —OP(O)(OR¹¹)₂, —NO₂, —NH₂, —NHR¹¹, —NR¹¹R¹¹, —N⁺(O)R¹¹R¹¹, —NHC(O)H, —NHC(O)R¹¹, —NR¹¹C(O)R¹¹, —NHC(O)NH₂, —NHC(O)NR¹¹R¹¹, —NR¹¹C(O)NHR¹¹, —SH, —SR¹¹, —S(O)H, —S(O)R¹¹, —SO₂R¹¹, —SO₂NH₂, —SO₂NHR¹¹, —SO₂NR¹¹R¹¹, —CF₃, —CHF₂, —CH₂F, —OCF₃, —OCHF₂, —CN, —CO₂H, —CO₂R¹¹, —CHO, —C(O)R¹¹, —C(O)NH₂, —C(O)NHR¹¹, —C(O)NR¹¹R¹¹, —CONHSO₂H, —C(O)NHSO₂R¹¹, —C(O)NR¹¹SO₂R¹¹, cyclic C₃-C₇ alkylamino, imidazolyl, piperazinyl, morpholinyl, thiomorpholinyl, piperidinyl, azepanyl, pyrrolidinyl and azetidinyl; wherein each of the groups cyclic C₃-C₇ alkylamino, imidazolyl, piperazinyl, morpholinyl, thiomorpholinyl, piperidinyl, azepanyl, pyrrolidinyl and azetidinyl are optionally substituted by one or more of the following groups: C₁₋₆ alkyl, C₂₋₆ alkenyl, C₂₋₆ alkynyl, C₃₋₇ cyclic alkyl, halo, —OH, —OR¹³, —OC(O)R¹³, —OC(O)NH₂, —OC(O)NHR¹³, —OC(O)NR¹³R¹³, —OP(O)(OH)₂, —OP(O)(OR¹³)₂, —NO₂, —NH₂, —NHR¹³, —NR¹³R¹³—N⁺(—O)R¹³R¹³, —NHC(O)H, —NHC(O)R¹³, —NR¹³C(O)R¹³, —NHC(O)NH₂, —NHC(O)NR¹³R¹³, —NR¹³C(O)NHR¹³, —SH, —SR¹³, —S(O)H, —S(O)R¹³, —SO₂R¹³, —SO₂NH₂, —SO₂NHR¹³, —SO₂NR¹³R¹³, —CF₃, —CH F₂, —CH₂F, —OCF₃, —OCHF₂, —CN, —CO₂H, —CO₂R¹³, —CHO, —C(O)R¹³, —C(O)NH₂, —C(O)NHR¹³, —C(O)NR¹³R¹³, —CONHSO₂H, —C(O)NHSO₂R¹³, and —C(O)NR¹³SO₂R¹³; wherein each R¹¹ and R¹³ is independently selected from a C₁₋₆ alkyl group, a C₂₋₆ alkenyl group, a C₂₋₆ alkynyl group and a C₃₋₇ cyclic alkyl group; and
(b) an optionally substituted aryl, and an optionally substituted heteroaryl group having up to 12 carbon atoms and having one or more heteroatoms in its ring system which are each independently selected from 0, N and S; and wherein the one or more optional substituents for each of said aryl and heteroaryl are each independently selected from the same optional substituents as those defined in (a) above for R⁹ and R¹⁰;
or
(c) R⁹ and R¹⁰ taken together can form a partially saturated or a fully unsaturated 5- or 6-membered ring of carbon atoms optionally including 1 to 3 heteroatoms selected from O, N and S, and the ring can be optionally substituted independently with 1 to 5 substituents selected from the same optional substituents as those defined in (a) above for R⁹ and R¹⁰.
(2). A compound of Formula I or a pharmaceutically acceptable salt thereof, wherein W, X, Y, and Z are all as defined above in (1), and wherein:
R¹, R², R³ and R⁴ are each independently selected from the following groups: H, halo, R, —OH, —OR, —OC(O)H, —OC(O)R, —OC(O)NH₂, —OC(O)NHR, —OC(O)NRR, —OP(O)(OH)₂, —OP(O)(OR)₂, —NO₂, —NH₂, —NHR, —NRR, —NHC(O)H, —NHC(O)R, —NRC(O)R, —NHC(O)NH₂, —NHC(O)NRR, —NRC(O)NHR, —SH, —SR, —S(O)H, —S(O)R, —SO₂R, —SO₂NH₂, —SO₂NHR, —SO₂NRR, —CF₃, —OCF₃, —OCHF₂, —CN, —C—CH, —C—CR, —CH═CHR, —CH═CRR, —CR═CHR, —CR═CRR, —CO₂H, —CO₂R, —CHO, —C(O)R, —C(O)NH₂, —C(O)NHR, —C(O)NRR, —CONHSO₂H, —CONHSO₂R, —CONRSO₂R, cyclic C₃-C₇ alkylamino, imidazolyl, C₁-C₆ alkylpiperazinyl, morpholinyl and thiomorpholinyl;
or R¹ and R² taken together, or R² and R³ taken together, or R³ and R⁴ taken together can form a saturated or a partially saturated or a fully unsaturated 5- or 6-membered ring of carbon atoms optionally including 1 to 3 heteroatoms selected from O, N and S, and the ring is optionally substituted independently with 1 to 4 substituents selected from R;
each R is independently selected from the following groups defined in paragraphs (a) and (b) below:
   (a) an optionally substituted C₁₋₆ alkyl group, an optionally substituted C₂₋₆ alkenyl group, an optionally substituted C₂₋₆ alkynyl group and an optionally substituted C₃₋₇ cyclic alkyl group; wherein the one or more optional substituents for each of said alkyl, alkenyl, alkynyl and cyclic alkyl groups are each independently selected from the following groups: halo, —OH, —OR⁵, —OC(O)R⁵, —OC(O)NH₂, —OC(O)NHR⁵, —OC(O)NR⁵R⁵, —OP(O)(OH)₂, —OP(O)(OR⁵)₂, —NO₂, —NH₂, —NHR⁵, —NR⁵R⁵, —N⁺(O⁻)R⁵R⁵, —NHC(O)H, —NHC(O)R⁵, —NR⁵C(O)R⁵, —NHC(O)NH₂, —NHC(O)NR⁵R⁵, —NR⁵C(O)NHR⁵, —SH, —SR⁵, —S(O)H, —S(O)R⁵, —SO₂R⁵, —SO₂NH₂, —SO₂NHR⁵, —SO₂NR⁵R⁵, —CF₃, —OCF₃, —OCHF₂, —CN, —CO₂H, —CO₂R⁵, —CHO, —C(O)R⁵, —C(O)NH₂, —C(O)NHR⁵, —C(O)NR⁵R⁵, —CONHSO₂H, —C(O)NHSO₂R⁵, —C(O)NR⁵SO₂R⁵, cyclic C₃-C₇ alkylamino, imidazolyl, piperazinyl, morpholinyl, thiomorpholinyl, piperidinyl, azepanyl, pyrrolidinyl and azetidinyl; wherein each of the groups imidazolyl, piperazinyl, morpholinyl, thiomorpholinyl, piperidinyl, azepanyl, pyrrolidinyl and azetidinyl are optionally substituted by one or more of the following groups: C₁₋₆ alkyl, C₂₋₆ alkenyl, C₂₋₆ alkynyl, C₃₋₇ cyclic alkyl, halo, —OH, —OR⁷, —OC(O)R⁷, —OC(O)NH₂—OC(O)NHR⁷, —OC(O)NR⁷R⁷, —OP(O)(OH)₂, —OP(O)(OR⁷)₂, —NO₂, —NH₂, —NHR⁷, —NR R⁷, —N⁺(O⁻) R⁷R⁷, —NHC(O)H, —NHC(O)R⁷, —NR⁷C(O)R⁷, —NHC(O)NH₂, —NHC(O)NR⁷R⁷, —NR⁷C(O)NHR⁷, —SH, —SR⁷, —S(O)H, —S(O)R⁷, —SO₂R⁷, —SO₂NH₂, —SO₂NHR⁷, —SO₂NR⁷R⁷, —CF₃, —OCF₃, —OCHF₂, —CN, —CO₂H, —CO₂R⁷, —CHO, —C(O)R⁷, —C(O)NH₂, —C(O)NHR⁷, —C(O)NR⁷R⁷, —CONHSO₂H, —C(O)NHSO₂R⁷, —C(O)NR⁷SO₂R⁷ an optionally substituted aryl, and an optionally substituted heteroaryl group having up to 12 carbon atoms and having one or more heteroatoms in its ring system which are each independently selected from O, N and S; and wherein the one or more optional substituents for each of said aryl and heteroaryl groups are each independently selected from the following groups: C₁₋₆ alkyl, C₂₋₆ alkenyl, C₂₋₆ alkynyl, C₃₋₇ cyclic alkyl, halo, —OH, —OR⁸, —OC(O)R⁸, —OC(O)NH₂, —OC(O)NHR⁸, —OC(O)NR⁸R⁸, —OP(O)(OH)₂, —OP(O)(OR⁸)₂, —NO₂, —NH₂, —NHR⁸, —NR⁸R⁸, —N⁺(O)R⁸R⁸, —NHC(O)H, —NHC(O)R⁸, —NR⁸C(O)R⁸, —NHC(O)NH₂, —NHC(O)NR⁸R⁸, —NR⁸C(O)NHR⁸, —SH, —SR⁸, —S(O)H, —S(O)R⁸, —SO₂R⁸, —SO₂NH₂, —SO₂NHR⁸, —SO₂NR⁸R⁸, —CF₃, —OCF₃, —OCHF₂, —CN, —CO₂H, —CO₂R⁸, —CHO, —C(O)R⁸, —C(O)NH₂, —C(O)NHR⁸, —C(O)NR⁸R⁸, —CONHSO₂H, —C(O)NHSO₂R⁸, and —C(O)NR⁸SO₂R⁸;
wherein each R⁵, R¹ and R⁸ is independently selected from a C₁₋₆ alkyl group, a C₂₋₆ alkenyl group. a C₂₋₆ alkynyl group and a C₃₋₇ cyclic alkyl group; and
   (b) an optionally substituted aryl, and an optionally substituted heteroaryl group having up to 12 carbon atoms and having one or more heteroatoms in its ring system which are each independently selected from O, N and S; and wherein the one or more optional substituents for each aryl and heteroaryl are each independently selected from the same optional substituents as those defined in (a) above for R;
R⁹ and R¹⁰ are each independently selected from the following groups defined in paragraphs (a) and (b) below:
   (a) H, an optionally substituted C₁₋₆ alkyl group, an optionally substituted C₂₋₆ alkenyl group, an optionally substituted C₂₋₆ alkynyl group, and an optionally substituted C₃₋₇ cyclic alkyl group; wherein the one or more optional substituents for each of said alkyl, alkenyl, alkynyl and cyclic alkyl are each independently selected from the following groups: halo, —OH, —OR¹¹, —OC(O)R¹¹, —OC(O)NH₂, —OC(O)NHR¹¹, —OC(O)NR¹¹R¹¹, —OP(O)(OH)₂, —OP(O)(OR¹¹)₂, —NO₂, —NH₂, —NHR¹¹, —NR¹¹R¹¹, —N⁺(O)R¹¹R¹¹, —NHC(O)H, —NHC(O)R¹¹, —NR¹¹C(O)R¹¹, —NHC(O)NH₂, —NHC(O)NR¹¹R¹¹, —NR¹¹C(O)NHR¹¹, —SH, —SR¹¹, —S(O)H, —S(O)R¹¹, —SO₂R¹¹, —SO₂NH₂, —SO₂NHR¹¹, —SO₂NR¹¹R¹¹, —CF₃, —OCF₃, —OCHF₂, —CN, —CO₂H, —CO₂R¹¹, —CHO, —C(O)R¹¹, —C(O)NH₂, —C(O)NHR¹¹, —C(O)NR¹¹R¹¹, —CONHSO₂H, —C(O)NHSO₂R¹¹, —C(O)NR¹¹SO₂R¹¹, cyclic C₃-C₇ alkylamino, imidazolyl, piperazinyl, morpholinyl, thiomorpholinyl, piperidinyl, azepanyl, pyrrolidinyl and azetidinyl; wherein each of the groups cyclic C₃-C₇ alkylamino, imidazolyl, piperazinyl, morpholinyl, thiomorpholinyl, piperidinyl, azepanyl, pyrrolidinyl and azetidinyl are optionally substituted by one or more of the following groups: C₁₋₆ alkyl, C₂₋₆ alkenyl, C₂₋₆ alkynyl, C₃₋₇ cyclic alkyl, halo, —OH, —OR¹³, —OC(O)R¹³, —OC(O)NH₂, —OC(O)NHR¹³, —OC(O)NR¹³R¹³, —OP(O)(OH)₂, —OP(O)(OR¹³)₂, —NO₂, —NH₂, —NHR¹³, —NR¹³R¹³—N⁺(O)R¹³R¹³, —NHC(O)H, —NHC(O)R¹³, —NR¹³C(O)R¹³, —NHC(O)NH₂, —NHC(O)NR¹³R¹³, —NR¹³C(O)NHR¹³, —SH, —SR¹³, —S(O)H, —S(O)R¹³, —SO₂R¹³, —SO₂NH₂, —SO₂NHR¹³, —SO₂NR¹³R¹³, —CF₃, OCF₃, —OCHF₂, —CN, —CO₂H, —CO₂R¹³, —CHO, —C(O)R¹³, —C(O)NH₂, —C(O)NHR¹³, —C(O)NR¹³R¹³, —CONHSO₂H, —C(O)NHSO₂R¹³, and —C(O)NR¹³SO₂R¹³; wherein each R¹¹ and R¹³ is independently selected from a C₁₋₆ alkyl group, a C₂₋₆ alkenyl group, a C₂₋₆ alkynyl group and a C₃₋₇ cyclic alkyl group; and
   (b) an optionally substituted aryl, and an optionally substituted heteroaryl group having up to 12 carbon atoms and having one or more heteroatoms in its ring system which are each independently selected from O, N and S; and wherein the one or more optional substituents for each of said aryl and heteroaryl are each independently selected from the same optional substituents as those defined in (a) above for R⁹ and R¹⁰; or
   (c) R⁹ and R¹⁰ taken together can form a partially saturated or a fully unsaturated 5- or 6-membered ring of carbon atoms optionally including 1 to 3 heteroatoms selected from O, N and S, and the ring can be optionally substituted independently with 1 to 5 substituents selected from the same optional substituents as those defined in (a) above for R⁹ and R¹⁰.
(3). A compound as defined in paragraph (1) or (2), wherein when R⁹ and R¹⁰ are each independently selected from the following groups: an optionally substituted C₁₋₆ alkyl group, an optionally substituted C₂₋₆ alkenyl group, an optionally substituted C₂₋₆ alkynyl group, and an optionally substituted C₃₋₇ cyclic alkyl group; then the one or more optional substituents for each of said alkyl, alkenyl, alkynyl and cyclic alkyl are each independently selected from the following groups: halo, —OH, —OR¹¹, —OC(O)R¹¹, —OC(O)NH₂, —OC(O)NHR¹¹, —OC(O)NR¹¹R¹¹, —OP(O)(OH)₂, —OP(O)(OR¹¹)₂, —NO₂, —NH₂, —NHR¹¹, —NR¹¹R¹¹, —N⁺(O)R¹¹R¹¹, —NHC(O)H, —NHC(O)R¹¹, —NR¹¹C(O)R¹¹, —NHC(O)NH₂, —NHC(O)NR¹¹R¹¹, —NR¹¹C(O)NHR¹¹, —SH, —SR¹¹, —S(O)H, —S(O)R¹¹, —SO₂R¹¹, —SO₂NH₂, —SO₂NHR¹¹, —SO₂NR¹¹R¹¹, —CF₃, —CHF₂, —CH₂F, —OCF₃, —OCHF₂, —CN, —CO₂H, —CO₂R¹¹, —CHO, —C(O)R¹¹, —C(O)NH₂, —C(O)NHR¹¹, —C(O)NR¹¹R¹¹, —CONHSO₂H, —C(O)NHSO₂R¹¹, and —C(O)NR¹¹SO₂R¹¹; wherein each R¹¹ is independently selected from a C₁₋₆ alkyl group, a C₂₋₆ alkenyl group, a C₂₋₆ alkynyl group and a C₃₋₇ cyclic alkyl group.
(4). A compound as defined in any one of paragraphs (1) to (3), wherein Z is N or N-oxide, such as N, W is CR¹, X is CR² and Y is CR³.
(5). A compound as defined in any one of paragraphs (1) to (3), wherein X is N or N-oxide, such as N, W is CR¹, Y is CR³ and Z is CR⁴.
(6). A compound as defined in any one of paragraphs (1) to (3), wherein X and Z are both N or N-oxide, such as N, W is CR¹ and Y is CR³.
(7). A compound as defined in any one of paragraphs (1) to (6), wherein R¹, R², R³ and R⁴, where present, are each independently selected from the group consisting of H, halo, optionally substituted C₁-C₆ alkyl, —O—R wherein R is optionally substituted C₁-C₆ alkyl, an optionally substituted aryl, such as substituted phenyl, and an optionally substituted heteroaryl group having up to 12 carbon atoms and having one or more heteroatoms in its ring system which are each independently selected from O, N and S.
(8). A compound as defined in any one of paragraphs (1) to (6), wherein R¹, R², R³ and R⁴, where present, are each independently selected from the group consisting of H, halo, optionally substituted C₁-C₆ alkyl, —O—R wherein R is selected from optionally substituted C₁-C₆ alkyl and optionally substituted aryl (such as phenyl), —NHR wherein R is optionally substituted aryl, an optionally substituted aryl, and an optionally substituted heteroaryl group having up to 12 carbon atoms and having one or more heteroatoms in its ring system which are each independently selected from O, N and S.
(9). A compound as defined in paragraph (8), wherein R¹, R², R³ and R⁴, where present, are each independently selected from the group consisting of H, halogen, —CF₃, —CHF₂, —OCF₃, —OCHF₂, C₁₋₆ alkyl, such as methyl, substituted aryl, substituted heteroaryl, —OR wherein R is optionally substituted aryl, and —NHR wherein R is optionally substituted aryl.
(10). A compound as defined in paragraph (8), wherein one or two of R¹, R², R³ and R⁴, where present, is H, and the others of R¹, R² R³ and R⁴ that are not H are independently selected from the group consisting of halogen, —CF₃, —CHF₂, —OCF₃, —OCHF₂, C₁₋₆ alkyl, such as methyl, substituted aryl, substituted heteroaryl, —OR wherein R is optionally substituted aryl, and —NHR wherein R is optionally substituted aryl.
(11). A compound as defined in any one of paragraphs (8) to (10), wherein R³ is present and selected from the group consisting of halogen, —OR wherein R is optionally substituted aryl, and —NHR wherein R is optionally substituted aryl.
(12). A compound as defined in paragraph (4), wherein Z is N or N-oxide, such as N, W is CR¹, X is CR² and Y is CR³, and R³ is selected from the group consisting of halogen, —O—R wherein R is optionally substituted aryl, and —NHR wherein R is optionally substituted aryl.
(13). A compound as defined in paragraph (4), wherein Z is N or N-oxide, such as N, W is CR¹, X is CR² and Y is CR³, R¹ is H, and one or both of R² and R³ are other than H, for example, both R² and R³ are other than H, or R² is H and R³ is other than H, or R³ is H and R² is other than H.
(14). A compound as defined in paragraph (13), wherein each of R² and R³ that is other than H is independently selected from the group consisting of halogen, optionally substituted C₁-C₆ alkyl, —OR wherein R is selected from optionally substituted C₁-C₆ alkyl and optionally substituted aryl, —NHR wherein R is optionally substituted aryl; an optionally substituted aryl, such as substituted phenyl, and an optionally substituted heteroaryl group.
(15). A compound as defined in paragraph (14), wherein each of R² and R³ that is other than H is independently selected from the group consisting of halogen, —CF₃, —CHF₂, —OCF₃, —OCHF₂, C₁₋₆ alkyl such as methyl, substituted aryl, substituted heteroaryl, —OR wherein R is optionally substituted aryl, and —NHR wherein R is optionally substituted aryl.
(16). A compound as defined in any one of paragraphs (1) to (6), wherein R¹ and R² taken together, or R² and R³ taken together, or R³ and R⁴ taken together form a saturated or a partially saturated or a fully unsaturated 5- or 6-membered ring of carbon atoms optionally including 1 to 3 heteroatoms selected from O, N or S and the ring is optionally substituted with 1 to 4 substituents independently selected from R, and those of R¹, R², R³ and R⁴ that are not part of the ring, are independently selected from: H, halo, optionally substituted C₁-C₆ alkyl, O—R wherein R is optionally substituted C₁-C₆ alkyl, an optionally substituted aryl and an optionally substituted heteroaryl group having up to 12 carbon atoms and having one or more heteroatoms in its ring system which are each independently selected from O, N and S.
(17). A compound as defined in any one of paragraphs (1) to (16), wherein R⁹ and R¹⁰ are independently selected from H, an optionally substituted C₁₋₆ alkyl group, an optionally substituted aryl, such as substituted phenyl, and an optionally substituted heteroaryl group having up to 12 carbon atoms and having one or more heteroatoms in its ring system which are each independently selected from O, N and S.
(18). A compound as defined in any one of paragraphs (1) to (17), wherein R⁹ and R¹⁰ are both H.
(19). A compound as defined in paragraph (4), wherein Z is N or N-oxide, such as N, W is CR¹, X is CR² and Y is CR³, and R⁹ and R¹⁰ are both H.
(20) A compound as defined in paragraph (19), wherein R¹, R² and R³ are each independently selected from the group consisting of H, halogen, —CF₃, —CHF₂, —OCF₃, —OCHF₂, C₁₋₆ alkyl, such as methyl, substituted aryl, substituted heteroaryl, —OR wherein R is optionally substituted aryl, and —NHR wherein R is optionally substituted aryl.
(21). A compound as defined in paragraph (19), wherein R¹, R² and R³ are each independently selected from the group consisting of H, halogen, C₁₋₆ alkyl, such as methyl, substituted aryl, and substituted heteroaryl.
(22). A compound as defined in paragraph (19), wherein one or two of R¹, R² and R³ is H, and the others of R¹, R² and R³ that are not H are independently selected from the group consisting of halogen, —CF₃, —CHF₂, —OCF₃, —OCHF₂, C₁₋₆ alkyl, such as methyl, substituted aryl, substituted heteroaryl, —OR wherein R is optionally substituted aryl, and —NHR wherein R is optionally substituted aryl.
(23). A compound as defined in paragraph (19), wherein R¹ is H, and one or both of R² and R³ is other than H, wherein each of R² and R³ that is not H is independently selected from the group consisting of halogen, —CF₃, —CHF₂, —OCF₃, —OCHF₂, C₁₋₆ alkyl, such as methyl, substituted aryl, substituted heteroaryl, —OR wherein R is optionally substituted aryl, and —NHR wherein R is optionally substituted aryl.
(24). A compound as defined in any one of paragraphs (19) to (23), wherein R³ is selected from the group consisting of halogen, —OR wherein R is optionally substituted aryl, and —NHR wherein R is optionally substituted aryl.
(25). A compound as defined in paragraph (19), wherein R¹ is H, and R² and R³ form a saturated or a partially saturated or a fully unsaturated 5- or 6-membered ring of carbon atoms optionally including 1 to 3 heteroatoms selected from O, N and S and the ring is optionally substituted with 1 to 4 substituents independently selected from R.
(26) A compound as defined in any one of paragraphs (1) to (6) wherein W is CR¹ and R¹ is selected from H, halo, —CHF₂, —CF₃ or methyl; and Z is N or N-oxide, such as N, X is CR² and Y is CR³; X is N or N-oxide, such as N, Y is CR³ and Z is CR⁴; or X and Z are both N or N-oxide, such as N, and Y is CR³.
(27) A compound as defined in any one of paragraphs (1) to (6) wherein W is CR¹ and R¹ is selected from H, halo, —CHF₂, —CF₃ or methyl; R⁹ and R¹⁰ are each H; and Z is N or N-oxide, such as N, X is CR² and Y is CR³; X is N or N-oxide, such as N, Y is CR³ and Z is CR⁴; or X and Z are both N or N-oxide, such as N, and Y is CR³.
(28) A compound as defined in any one of paragraphs (1) to (6) wherein W is CR¹ and R¹ is selected from H, halo, —CHF₂, —CF₃ or methyl; R⁹ and R¹⁰ are each H; Z is N or N-oxide, such as N, X is CR² and Y is CR³; and R² and R³ are each independently selected from: H, halogen, —CF₃, —CHF₂, —OCF₃, —OCHF₂, —NO₂, C₁₋₆ alkyl, such as methyl, optionally substituted aryl, optionally substituted heteroaryl, —OR wherein R is optionally substituted aryl, and —NHR wherein R is optionally substituted aryl; or R² and R³ taken together can form a saturated or a partially saturated or a fully unsaturated 5- or 6-membered ring of carbon atoms and the ring is optionally substituted with 1 to 4 substituents independently selected from R.
(29) A compound as defined in any one of paragraphs (1) to (6) wherein W is CR¹ and R¹ is selected from H, halo, —CHF₂, —CF₃ or methyl.
(30) A compound as defined in paragraphs (1) to (6) and (26) to (29) wherein W is CR¹ and R¹ is H, F, —CHF₂, —CF₃ or methyl.
(31) A compound as defined in paragraph (1) to (6) and (26) or (30) wherein W is CR¹ and R¹ is H or F.
(32) A compound as defined in any one of paragraphs (1) to (6) and (26) to (30) wherein W is CR¹ and R¹ is H or methyl.
(33) A compound as defined in any one of paragraphs (1) to (6) and (26) to (32) wherein W is CR¹ and R¹ is H.
(34) A compound as defined any one of paragraphs (1) to (6) and (29) to (33) wherein R⁹ and R¹⁰ are independently selected from H, an optionally substituted C₁₋₆ alkyl group, an optionally substituted aryl, such as substituted phenyl, and an optionally substituted heteroaryl group having up to 12 carbon atoms and having one or more heteroatoms in its ring system which are each independently selected from O, N and S.
(35) A compound as defined in any one of paragraphs (1) to (6) and (29) to (34) wherein R⁹ and R¹⁰ are each H.
(36) A compound as defined in any one of paragraphs (26) to (35), wherein Z is N or N-oxide, such as N, X is CR² and Y is CR³.
(37) A compound as defined in any one of paragraphs (26) to (35), wherein X is N or N-oxide, such as N, Y is CR³ and Z is CR⁴.
(38) A compound as defined in any one of paragraphs (26) to (35), wherein X and Z are both N or N-oxide, such as N, and Y is CR³.
(39) A compound as defined in any one of paragraphs (1) to (6) and (29) to (36) wherein R² and R³ are each independently selected from: H, halogen, —CF₃, —CHF₂, —OCF₃, —OCHF₂, —NO₂, C₁₋₆ alkyl, such as methyl, optionally substituted aryl, optionally substituted heteroaryl, —OR wherein R is optionally substituted aryl, and —NHR wherein R is optionally substituted aryl; or R² and R³ taken together can form a saturated or a partially saturated or a fully unsaturated 5- or 6-membered ring of carbon atoms and the ring is optionally substituted with 1 to 4 substituents independently selected from R.
(40) A compound as defined in any one of paragraphs (1) to (6), (28) to (36), and (39) wherein R² and R³ are each independently selected from: H, halogen, —CF₃, —CHF₂, —OCF₃, —OCHF₂, —NO₂, C₁₋₆ alkyl, such as methyl, optionally substituted aryl, optionally substituted heteroaryl, —OR wherein R is optionally substituted aryl, and —NHR wherein R is optionally substituted aryl; or R² and R³ taken together can form a saturated or a partially saturated or a fully unsaturated 6-membered ring of carbon atoms and the ring is optionally substituted with 1 to 4 substituents independently selected from R.
(41) A compound as defined in any one of paragraphs (1) to (6), (28) to (36), (39), and (40) wherein R² and R³ are each independently selected from: H, halogen, —CF₃, —CHF₂, —OCF₃, —OCHF₂, —NO₂, C₁₋₆ alkyl, such as methyl, optionally substituted aryl, optionally substituted heteroaryl, —OR wherein R is optionally substituted aryl, and —NHR wherein R is optionally substituted aryl; or R² and R³ taken together can form a saturated or a partially saturated or a fully unsaturated 6-membered ring of carbon atoms.
(42) A compound as defined in any one of paragraphs (1) to (6), (28) to (36), and (39) to (41) wherein R² and R³ are each independently selected from: H, halo, —CF₃, —CHF₂, —NO₂, and methyl.
(43) A compound as defined in any one of paragraphs (1) to (6), (28) to (36), and (39) to (42) wherein R² and R³ are each independently selected from: H, halo, —CF₃, and —NO₂.
(44) A compound as defined in any one of paragraphs (1) to (6), (28) to (35), and (38) to (43) wherein one of R² and R³ is H and the other is a group other than H.
(45) A compound as defined in any one of paragraphs (1) to (3), wherein W is CR¹ and R¹ is H or F; R⁹ and R¹⁰ are each H; Z is N or N-oxide, such as N, X is CR² and Y is CR³; and R² and R³ are each independently selected from: H, halo, —CF₃, —CHF₂, —NO₂, and methyl.
(45) A compound as defined in any one of paragraphs (1) to (3), wherein W is CR¹ and R¹ is H; R⁹ and R¹⁰ are each H; Z is N or N-oxide, such as N, X is CR² and Y is CR³; and R² and R³ are each independently selected from: H, halo, —CF₃, and —NO₂.
(46) A compound as defined in any one of paragraphs (1) to (3), (26), (27), (29) to (35) and (37), wherein X is N or N-oxide, such as N, Y is CR³ and Z is CR⁴; and R³ and R⁴ are each independently selected from the group of substituents listed for R² and R³ in any one of paragraphs (39) to (43).
(47) A compound as defined in any one of paragraphs (1) to (3), X and Z are both N or N-oxide, such as N, and Y is CR³; and R³ is selected from the group of substituents listed for R² and R³ in any one of paragraphs (39) to (43).
(46). A compound as defined in paragraph (1), selected from the group consisting of:
5-Bromo-4,6-dimethylisoxazolo[5,4-b]pyridin-3-amine (1)
Isoxazolo[5,4-b]pyridin-3-amine (2)
5-Chloro-4,6-dimethylisoxazolo[5,4-b]pyridin-3-amine (3)
4,6-Dimethylisoxazolo[5,4-b]pyridin-3-amine (4)
4,5,6-Trimethylisoxazolo[5,4-b]pyridin-3-amine (5)
5-Bromoisoxazolo[5,4-b]pyridin-3-amine (6)
6-Methylisoxazolo[5,4-b]pyridin-3-amine (7)
5-Chloroisoxazolo[5,4-b]pyridin-3-amine (8)
Isoxazolo[5,4-b]quinolin-3-amine (9)
5,6,7,8-Tetrahydroisoxazolo[5,4-b]quinolin-3-amine (10)
6-Chloroisoxazolo[5,4-b]pyridin-3-amine (11)
Isoxazolo[5,4-d]pyrimidin-3-amine (12)
5-Fluoroisoxazolo[5,4-b]pyridin-3-amine (14)
6-Phenylisoxazolo[5,4-b]pyridin-3-amine (15)
5-Iodoisoxazolo[5,4-b]pyridin-3-amine (16)
Isoxazolo[4,5-c]pyridin-3-amine (17)
N⁶,N⁶-Dimethylisoxazolo[5,4-b]pyridine-3,6-diamine (18)
N⁴,N⁴-Dimethylisoxazolo[5,4-b]pyridine-3,4-diamine (19)
5-(3-Methoxyphenyl)isoxazolo[5,4-b]pyridin-3-amine (22)
5-(2-Methoxyphenyl)isoxazolo[5,4-b]pyridin-3-amine (23)
5-Phenylisoxazolo[5,4-b]pyridin-3-amine (24)
5-(Pyridin-3-yl)isoxazolo[5,4-b]pyridin-3-amine (26)
5-(Pyridin-4-yl)isoxazolo[5,4-b]pyridin-3-amine (27)
2-(3-Aminoisoxazolo[5,4-b]pyridin-5-yl)phenol (28)
4-(3-Aminoisoxazolo[5,4-b]pyridin-5-yl)phenol (29)
5-(4-Fluorophenyl)isoxazolo[5,4-b]pyridin-3-amine (30)
5-(3-Fluorophenyl)isoxazolo[5,4-b]pyridin-3-amine (31)
5-(2,4-difluorophenyl)isoxazolo[5,4-b]pyridin-3-amine (32)
5-(3,5-Difluoro-2-methoxyphenyl)isoxazolo[5,4-b]pyridin-3-amine (33)
5-(2,4-Dichlorophenyl)isoxazolo[5,4-b]pyridin-3-amine (34)
5-(2,3,4-Trichlorophenyl)isoxazolo[5,4-b]pyridin-3-amine (35)
5-(4-(Trifluoromethylphenyl)isoxazolo[5,4-b]pyridin-3-amine (36)
5-(3-Aminophenyl)isoxazolo[5,4-b]pyridin-3-amine (37)
Methyl 3-(3-aminoisoxazolo[5,4-b]pyridin-5-yl)benzoate (38)
5-(6-Fluoropyridin-3-yl)isoxazolo[5,4-b]pyridin-3-amine (39)
5-(2-Chloro-4-(trifluoromethyl)phenyl)isoxazolo[5,4-b]pyridin-3-amine (40)
6-Methoxyisoxazolo[5,4-b]pyridin-3-amine (41)
6-Chloro-4-methylisoxazolo[5,4-b]pyridin-3-amine (42)
Isoxazolo[5,4-b]pyridine-3,6-diamine (43)
5-Methylisoxazolo[5,4-b]pyridin-3-amine (44)
5,6-Dimethylisoxazolo[5,4-b]pyridin-3-amine (45)
6-Methyl-4-(trifluoromethyl)isoxazolo[5,4-b]pyridin-3-amine (46)
6-(Trifluoromethyl)isoxazolo[5,4-b]pyridin-3-amine (47)
6-Isopropylisoxazolo[5,4-b]pyridin-3-amine (48)
5-Nitroisoxazolo[5,4-b]pyridin-3-amine (49)
Ethyl 3-amino-6-(trifluoromethyl)isoxazolo[5,4-b]pyridine-5-carboxylate (50)
4-Methoxyisoxazolo[5,4-b]pyridin-3-amine (51)
5-(Difluoromethoxy)-4,6-dimethylisoxazolo[5,4-b]pyridin-3-amine (52)
Ethyl 3-amino-6-methylisoxazolo[5,4-b]pyridine-5-carboxylate (53)
Ethyl 3-amino-6-(difluoromethyl)isoxazolo[5,4-b]pyridine-5-carboxylate (54)
5-Fluoro-6-morpholinoisoxazolo[5,4-b]pyridin-3-amine (55)
6-(Furan-2-yl)isoxazolo[5,4-b]pyridin-3-amine (58)
6,7,8,9-Tetrahydro-5H-cyclohepta[b]isoxazolo[4,5-e]pyridin-3-amine (60)
6,6-Dimethyl-5,6,7,8-tetrahydroisoxazolo[5,4-b]quinolin-3-amine (61)
7,8-Dihydro-5H-isoxazolo[5,4-b]pyrano[3,4-e]pyridin-3-amine (62)
6-(Methylthio)isoxazolo[5,4-d]pyrimidin-3-amine (63)
6-Methylisoxazolo[5,4-d]pyrimidin-3-amine (64)
6-Chloro-5-fluoroisoxazolo[5,4-b]pyridin-3-amine (66)
5,6-Dichloroisoxazolo[5,4-b]pyridin-3-amine (67)
6-Chloro-4-(trifluoromethyl)isoxazolo[5,4-b]pyridin-3-amine (68)
5-(3-Methoxyprop-1-yn-1-yl)isoxazolo[5,4-b]pyridin-3-amine (69)
6-(4-Fluorophenyl)isoxazolo[5,4-b]pyridin-3-amine (71)
6-(2,4-Difluorophenyl)isoxazolo[5,4-b]pyridin-3-amine (73)
6-(2-Thienyl)isoxazolo[5,4-b]pyridin-3-amine (74)
6-(Methylthio)isoxazolo[5,4-b]pyridin-3-amine (79)
6-(Methylsulfonyl)isoxazolo[5,4-b]pyridin-3-amine (80)
Methyl 3-aminoisoxazolo[5,4-b]pyridine-6-carboxylate (82)
6-Phenoxyisoxazolo[5,4-b]pyridin-3-amine (83)
6-(2-Chlorophenoxy)isoxazolo[5,4-b]pyridin-3-amine (84)
6-(3-Chlorophenoxy)isoxazolo[5,4-b]pyridin-3-amine (85)
6-(4-Chlorophenoxy)isoxazolo[5,4-b]pyridin-3-amine (86)
6-(2-(Trifluoromethoxy)phenoxy)isoxazolo[5,4-b]pyridin-3-amine (87)
6-(3-(Trifluoromethoxy)phenoxy)isoxazolo[5,4-b]pyridin-3-amine (88)
6-(4-(Trifluoromethoxy)phenoxy)isoxazolo[5,4-b]pyridin-3-amine (89)
6-(2-Methoxyphenoxy)isoxazolo[5,4-b]pyridin-3-amine (90)
6-(3-Methoxyphenoxy)isoxazolo[5,4-b]pyridin-3-amine (91)
6-(4-Methoxyphenoxy)isoxazolo[5,4-b]pyridin-3-amine (92)
6-(3-(Trifluoromethyl)phenoxy)isoxazolo[5,4-b]pyridin-3-amine (93)
N⁶-Phenylisoxazolo[5,4-b]pyridine-3,6-diamine (94)
N⁶-(3-Methoxyphenyl)isoxazolo[5,4-b]pyridine-3,6-diamine (95) and
N⁶-(4-Methoxyphenyl)isoxazolo[5,4-b]pyridine-3,6-diamine (96),
   and pharmaceutically acceptable salts thereof.
   (47) A compound as defined in paragraph (1), selected from the group consisting of:
6-Chloro-5-fluoroisoxazolo[5,4-b]pyridin-3-amine (66)
5-Bromoisoxazolo[5,4-b]pyridin-3-amine (6)
5-Chloroisoxazolo[5,4-b]pyridin-3-amine (8)
6-Chloroisoxazolo[5,4-b]pyridin-3-amine (11)
5-Iodoisoxazolo[5,4-b]pyridin-3-amine (16)
5-Nitroisoxazolo[5,4-b]pyridin-3-amine (49)
5,6-Dichloroisoxazolo[5,4-b]pyridin-3-amine (67)
5-Chloro-4,6-dimethylisoxazolo[5,4-b]pyridin-3-amine (3)
4,6-Dimethylisoxazolo[5,4-b]pyridin-3-amine (4)
4,5,6-Trimethylisoxazolo[5,4-b]pyridin-3-amine (5)
6-Methylisoxazolo[5,4-b]pyridin-3-amine (7)
5-Phenylisoxazolo[5,4-b]pyridin-3-amine (24)
5-Bromo-4,6-dimethylisoxazolo[5,4-b]pyridin-3-amine (1)
Isoxazolo[5,4-b]quinolin-3-amine (9)
5-(4-Fluorophenyl)isoxazolo[5,4-b]pyridin-3-amine (30)
5,6,7,8-Tetrahydroisoxazolo[5,4-b]quinolin-3-amine (10)
Isoxazolo[5,4-d]pyrimidin-3-amine (12)
5-Fluoroisoxazolo[5,4-b]pyridin-3-amine (14)
6-Phenylisoxazolo[5,4-b]pyridin-3-amine (15)
6-(2-Thienyl)isoxazolo[5,4-b]pyridin-3-amine (74)
6-Methoxyisoxazolo[5,4-b]pyridin-3-amine (41)
6-(Trifluoromethyl)isoxazolo[5,4-b]pyridin-3-amine (47)
6-Chloro-4-methylisoxazolo[5,4-b]pyridin-3-amine (42)
5,6-Dimethylisoxazolo[5,4-b]pyridin-3-amine (45)
5-Methylisoxazolo[5,4-b]pyridin-3-amine (44)
6-(2-Chlorophenoxy)isoxazolo[5,4-b]pyridin-3-amine (84)
6-(4-(Trifluoromethoxy)phenoxy)isoxazolo[5,4-b]pyridin-3-amine (89)
N⁶-(3-Methoxyphenyl)isoxazolo[5,4-b]pyridine-3,6-diamine (95),
   and pharmaceutically acceptable salts thereof.
   (48) A compound as defined in paragraph (1), selected from the group consisting of:
6-Chloro-5-fluoroisoxazolo[5,4-b]pyridin-3-amine (66)
5-Bromoisoxazolo[5,4-b]pyridin-3-amine (6)
5-Chloroisoxazolo[5,4-b]pyridin-3-amine (8)
6-Chloroisoxazolo[5,4-b]pyridin-3-amine (11)
5-Iodoisoxazolo[5,4-b]pyridin-3-amine (16)
5-Nitroisoxazolo[5,4-b]pyridin-3-amine (49)
5,6-Dichloroisoxazolo[5,4-b]pyridin-3-amine (67),
   and pharmaceutically acceptable salts thereof.
   (49) A compound as defined in paragraph (1), wherein the compound is 6-Chloro-5-fluoroisoxazolo[5,4-b]pyridin-3-amine (66) or pharmaceutically acceptable salts thereof.

In various embodiments relating to the treatment, prevention or kit, the dual inhibitor of IDO1 and TDO is 6-Chloro-5-fluoroisoxazolo[5,4-b]pyridin-3-amine (66) or a pharmaceutically acceptable salt thereof and the refractory cancer being treated or prevented is refractory to a platinum-based chemotherapeutic agent. For example, in various embodiments, the platinum-based chemotherapeutic agent is selected from: carboplatin, cisplatin, lobaplatin, oxaliplatin, picoplatin, nedaplatin, phenanthriplatin and/or satraplatin. In a further example, the platinum-based chemotherapeutic agent is selected from: cisplatin, carboplatin and/or oxaliplatin.

In various embodiments relating to the treatment, prevention or kit, the dual inhibitor of IDO1 and TDO is 6-Chloro-5-fluoroisoxazolo[5,4-b]pyridin-3-amine (66) or a pharmaceutically acceptable salt thereof, and the refractory cancer being treated or prevented is refractory to a platinum-based chemotherapy. For example, in various embodiments, the platinum-based chemotherapy comprises a step of administering a chemotherapeutic agent selected from: carboplatin, cisplatin, lobaplatin, oxaliplatin, picoplatin, nedaplatin, phenanthriplatin and/or satraplatin.

In various embodiments relating to the treatment, prevention or kit, the dual inhibitor of IDO1 and TDO is 6-Chloro-5-fluoroisoxazolo[5,4-b]pyridin-3-amine (66) or a pharmaceutically acceptable salt thereof, and the cancer being treated or prevented is selected from lung cancer, pancreatic cancer, breast cancer and ovarian cancer and is refractory to a platinum-based chemotherapeutic agent. For example, in various embodiments, the platinum-based chemotherapeutic agent is selected from: carboplatin, cisplatin, lobaplatin, oxaliplatin, picoplatin, nedaplatin, phenanthriplatin and/or satraplatin. In a further example, the platinum-based chemotherapeutic agent is selected from: cisplatin, carboplatin and/or oxaliplatin.

In various embodiments relating to the treatment, prevention or kit, the dual inhibitor of IDO1 and TDO is 6-Chloro-5-fluoroisoxazolo[5,4-b]pyridin-3-amine (66) or a pharmaceutically acceptable salt thereof, and the cancer being treated or prevented is selected from lung cancer, pancreatic cancer, breast cancer and ovarian cancer and is refractory to a platinum-based chemotherapy. For example, in various embodiments, the platinum-based chemotherapy comprises a step of administering a chemotherapeutic agent selected from: carboplatin, cisplatin, lobaplatin, oxaliplatin, picoplatin, nedaplatin, phenanthriplatin and/or satraplatin.

In various embodiments, the dual inhibitor of IDO1 and TDO has a cellular IDO1 IC₅₀ of less than 100 M as determined by a cell-based assay for IDO1 inhibition; and has a cellular TDO IC₅₀ of less than 100 M as determined by a cell-based assay for TDO inhibition. In various embodiment, the dual inhibitor of IDO1 and TDO described herein has a cellular IDO1 IC₅₀ of less 10 M as determined by a cell-based assay for IDO1 inhibition; and has a cellular TDO IC₅₀ of less than 10 M as determined by a cell-based assay for TDO inhibition. In various embodiment, the dual inhibitor of IDO1 and TDO described herein has a cellular IDO1 IC₅₀ of less 1 M as determined by a cell-based assay for IDO1 inhibition; and has a cellular TDO IC₅₀ of less than 1 M as determined by a cell-based assay for TDO inhibition.

In various embodiments, the dual inhibitor of IDO1 and TDO is formulated with an appropriate pharmaceutically acceptable carrier, for example an excipient, diluent, auxiliary or combinations thereof.

In various embodiments, the dual inhibitor of IDO1 and TDO is formulated or provided as a pharmaceutical composition comprising the dual inhibitor of IDO1 and TDO and a pharmaceutically acceptable carrier.

In various embodiments, the dual inhibitor of IDO1 and TDO is formulated or provided as a pharmaceutical composition comprising the dual inhibitor of IDO1 and TDO and a pharmaceutically acceptable carrier, for oral administration.

In various embodiments, the dual inhibitor of IDO1 and TDO is administered orally.

In various embodiments, the method, use or dual inhibitor for preventing the development of refractory cancer or a refractory cancer cell, comprises administering to a subject or the cell the dual inhibitor of IDO1 and TDO in combination with the anticancer agent and/or cancer therapy which the cancer or cancer cell is to be prevented from becoming refractory to, and optionally, wherein the method, use or dual inhibitor further comprises treating the cancer or cancer cell at risk of becoming refractory to the anticancer agent and/or therapy.

In various embodiments, the method, use or dual inhibitor for preventing the development of refractory cancer that is refractory to an anticancer agent and/or cancer therapy, comprises administering to a subject in need thereof the dual inhibitor of IDO1 and TDO in combination with the anticancer agent and/or cancer therapy. In various embodiments, the dual inhibitor of IDO1 and TDO and the anticancer agent and/or cancer therapy are administered simultaneously, sequentially, or separately. In various embodiments, the dual inhibitor of IDO1 and TDO and the anticancer agent and/or cancer therapy are administered as a single formulation or as separate formulations.

In various embodiments, the method, use or dual inhibitor further comprises a step of administering one or more additional agents selected from the group consisting of: anticancer agents, immune-modulating agents such as anticancer vaccines, modulators of immune checkpoint proteins, adoptive T cell immunotherapies (for example chimeric antigen receptor T cells (CART cells)), and radiotherapy, and wherein the additional agent is administered either before, during or after administration of the dual inhibitor of IDO1 and TDO. In various embodiments, the one or more additional agent is an immune-modulating agent selected from: an inhibitor of CTLA4, an anti-PD-1 antibody or an anti-PD-L1 antibody. For example, in various embodiments, the one or more additional agent is selected from: Ipilimumab, Cemiplimab, Nivolumab, Pembrolizumab, Atezolizumab, Avelumab and Durvalumab.

In various embodiments, the one or more additional agent is administered simultaneously, sequentially, or separately with the dual inhibitor of IDO1 and TDO. In various embodiments, the dual inhibitor of IDO1 and TDO and one or more additional agents are administered as a single formulation or as separate formulations.

Whenever a range is given in the specification, for example, a temperature range, a time range, or a composition range, all intermediate ranges and subranges, as well as all individual values included in the ranges given are intended to be included in the disclosure. It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

Other aspects of the invention may include suitable combinations of embodiments disclosed herein. Also, as will be appreciated by one of skill in the art, features and preferred embodiments of one aspect of the invention will also pertain to other aspects of the invention.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

While the invention is broadly as defined above, it is not limited thereto and also includes embodiments of which the following description provides examples. The invention will now be described in more detail.

### BRIEF DESCRIPTION OF THE FIGURES

The present invention will be described with reference to the accompanying Figures, in which:

FIG. 1 shows graphs demonstrating the % cell viability of human-derived cisplatin-sensitive lung cancer cell lines B, S, A and cisplatin-resistant lung cancer cell lines BC, SC and ALC after incubation with AT-0174.

FIGS. 2A-2C show antitumor activity of the selective IDO1 inhibitor epacadostat or the dual inhibitor AT-0174 in a syngeneic mouse model of lung cancer. (A) LLC-CR possessed higher basal level of T-reg (CD4⁺ CD25⁺) and lower natural killer (NK; CD3⁻CD49b⁺) cell populations when compared with parental tumor. (B) Anti-tumor activity of epacadostat or AT-0174 in parental vs, cisplatin-resistant cell allografts. Tumor growth was significantly reduced in the cisplatin-resistant tumor group treated with EPA or AT-0174 (*P=0.042, **P=0.014; using one way Anova with Tukey's multiple comparison test; n=5). (C) Significant reduction in tumor weight was observed in mice treated with AT-0174; *p=0,002 (n=5).

FIG. 3 shows that both epacadostat (EPA) and AT-0174 (AT) suppressed T reg and increased NK cell activity in CR tumor; however the dual inhibitor AT-0174 was more potent at enhancing immune activity in the tumor microenvironment. Higher NK cell activity and lower Treg populations were found in AT-0174 treatment group when compared to epacadostat (p=0.035 and p=0.02, respectively (n=5)).

FIGS. 4A-4C show kynurenine (KYN) and tryptophan (TRP) levels in mouse serum following treatment with epacadostat or AT-0174. (A) LLC-CR mice displayed a higher basal KYN/TRP ratio. Importantly, treatment AT-0174 in mice bearing cisplatin-resistant tumors significantly suppressed the KYN/TRP ratio (A; *p=0.002; n=5), and KYN (B; **p=0.03), and increased in TRP (C; ***p=0.02).

FIGS. 5A-5C show graphs demonstrating that AT-0174 increased survival to a greater extent than the selective agent (FIG. 5A), the anti-PD1 antibody given alone had a similar effect on survival as AT-0174 or epacadostat, but it only appeared to provide marked synergy with AT-0174 (FIG. 5B) with a lesser synergistic effect evident with epacadostat (FIG. 5C).

FIG. 6 shows body weight of mice administered either vehicle, oxaliplatin or oxaliplatin plus AT-1074 in a mouse PanO2 syngeneic model of pancreatic cancer.

FIG. 7 shows tumor volume of mice administered either vehicle, oxaliplatin or oxaliplatin plus AT-1074 in a mouse PanO2 syngeneic model of pancreatic cancer.

FIG. 8 shows relative tumor volume of mice administered either vehicle, oxaliplatin or oxaliplatin plus AT-1074 in a mouse PanO2 syngeneic model of pancreatic cancer.

FIG. 9 shows changes in immune cell populations within PanO2 Tumors from mice administered either vehicle, oxaliplatin or oxaliplatin plus AT-1074.

### DETAILED DESCRIPTION OF THE INVENTION

### Definitions

As used herein, “comprising” is synonymous with “including,” “containing,” or “characterized by,” and is inclusive or open-ended and does not exclude additional, unrecited elements or method steps. When interpreting each statement in this specification and claims that includes the term “comprising”, features other than that or those prefaced by the term may also be present. Related terms such as “comprise” and “comprises” are to be interpreted in the same manner.

As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

Unless otherwise stated, the singular forms “a,” “an,” and “the” include the plural reference.

In the disclosure and the claims, “and/or” means additionally or alternatively.

Moreover, any use of a term in the singular also encompasses plural forms.

It is to be recognised that certain compounds of the present invention may exist in one or more different enantiomeric or diastereomeric forms. It is to be understood that the enantiomeric or diastereomeric forms are included in the above aspects of the invention.

The term “halo” or “halogen” as used throughout the specification is to be taken as meaning a fluoro, chloro, bromo or iodo group.

It is to be understood that where variables of the Formula I as defined above are optionally substituted by one or more imidazolyl, piperazinyl, morpholinyl, thiomorpholinyl, piperidinyl, azepanyl, pyrrolidinyl and azetidinyl groups that the linkage to the relevant variable may be through either one of the available nitrogen or carbon ring atoms of these groups.

It is to be understood that the term “heteroaryl” includes both monocyclic and bicyclic ring systems, unless the context requires otherwise.

It is to be understood that the term “aryl” means an aromatic hydrocarbon such as phenyl or naphthyl.

It is to be understood that where a group is qualified as being “optionally substituted”, this means that the group can be either (a) unsubstituted or (b) substituted by the defined substituents.

It is to be understood that where reference is made throughout the specification to a C₁-C₆ alkyl or C₂-C₆ alkenyl group, these groups may be unbranched or branched. For example, it is intended that reference to a C₁-C₆ alkyl would include a tert-butyl (Me)₃C— group.

A “subject” refers to a warm-blooded animal. “Warm blooded animal” means any member of the Mammalia class including, but not limited to humans, non-human primates such as chimpanzees and other apes and monkey species, farm animals such as cattle, horses, sheep, goats, deer, and swine; domestic animals such as rabbits, dogs and cats; laboratory animals including rodents, such as rats, mice and guinea pigs, and the like. Preferably, the subject is a human.

As used herein, the term “chemotherapy” means the use of one or more chemotherapeutic agent in the treatment or prevention of cancer, for example to kill cancer cells or shrink tumours.

As used herein, the term “radiotherapy” means the use of high-energy radiation from x-rays, gamma rays, neutrons, protons, and other sources to kill cancer cells and shrink tumors. Radiation may come from a machine outside the body (external-beam radiation therapy), or it may come from radioactive material placed in the body near cancer cells (internal radiation therapy). Systemic radiotherapy uses a radioactive substance, such as a radiolabelled monoclonal antibody, that travels in the blood to tissues throughout the body. The terms irradiation and radiation therapy have the same meaning.

The expressions “treating refractory cancer”, “treatment of refractory cancer”, “treating a refractory cancer cell” and “treatment of a refractory cancer cell”, and similar expressions, as used herein, relate generally to treatment in which a desired therapeutic effect is achieved. The therapeutic effect may, for example, be inhibition, reduction, amelioration, halt, or prevention (for example of growth) of the refractory cancer. For example, in some embodiments, the therapeutic effect may be inhibiting, reducing, preventing, or halting cancer or cancer cells refractory to a platinum based chemotherapy (including inhibiting, reducing, preventing, or halting cancer or cancer cells refractory to a platinum based chemotherapeutic agent). The expressions include methods that produce one or more anticancer effects. “Anticancer effects” include, but are not limited to, anti-tumor effects, the response rate, the time to disease progression and the overall survival rate. “Anti-tumor” effects include but are not limited to inhibition of tumor growth, tumor growth delay, regression of tumor, shrinkage of tumor, increased time to regrowth of tumor on cessation of treatment, slowing of disease progression.

The expressions “preventing refractory cancer”, “preventing the development of refractory cancer”, “preventing the development of a refractory cancer cell”, and “preventing the development of cancer or a cancer cell refractory to an anticancer agent and/or cancer therapy”, and similar expressions, as used herein, mean preventing or inhibiting the development of cancer or a cancer cell that is refractory to a specific anticancer agent and/or cancer therapy from a cancer or cancer cell that is not refractory to said anticancer agent and/or therapy, or preventing or reducing the likelihood of the occurrence or recurrence of the refractory cancer or refractory cancer cell, and/or delaying development of the refractory cancer or refractory cancer cell. For example, in some embodiments, the expressions relate to preventing or inhibiting the development of refractory cancer that is refractory to a specific platinum based chemotherapeutic agent from a cancer that is not refractory to said specific platinum based chemotherapeutic agent. The expressions also include delaying the onset or recurrence of refractory cancer or delaying the occurrence or recurrence of the symptoms of refractory cancer.

“Therapeutically effective amount” means an amount of a compound that, when administered to a subject for treating or preventing a refractory cancer, is sufficient to effect such treatment or prevention for the refractory cancer. The “effective amount” will vary depending on the refractory cancer to be treated, the compound to be administered, the severity of the refractory cancer treated, the age and relative health of the subject, the route and form of administration, whether the treatment is monotherapy or combination therapy, the judgement of the attending clinician, and other factors.

The phrase “refractory cancer” or “refractory to (an anticancer agent and/or cancer therapy)”, and similar terms, as used herein means that a particular cancer or cancer cell either fails to respond favourably to or is resistant to a specific anticancer agent and/or cancer therapy, or alternatively, recurs or relapses after responding favourably to a specific anticancer agent and/or cancer therapy. Accordingly, for example, a cancer “refractory to a platinum based chemotherapeutic agent” means a cancer that fails to respond favourably to or is resistant to a platinum based chemotherapeutic agent, or recurs or relapses after responding favourably to such treatment.

Methods of determining or assessing a refractory cancer or a refractory cancer cell will be apparent to those skilled in the art. For example, to detect or identify refractory cancer or cancer that is refractory to (a treatment), subjects undergoing a cancer treatment or therapy can be carefully monitored for signs of resistance, non-responsiveness, recurrence or relapse of the cancer. This can be accomplished by monitoring the subject's cancer's response to the treatment. The response, lack of response, recurrence or relapse of the cancer to a therapy, for example, the initial therapy, can be determined by any suitable method practiced in the art. For example, this can be accomplished by assessing one or more anticancer effects, the lack of which indicates that the cancer is not responding to the cancer therapy, or that a relapse has occurred. For example, this can be accomplished by the assessment of tumor size and number. An increase in tumor size or, alternatively, tumor number, indicates that the tumor is not responding to the chemotherapy, or that a relapse has occurred. The determination can be made according to the “RECIST” criteria as described in detail in Eisenhauer, E A et al. European journal of cancer (Oxford, England: 7990) vol. 45,2 (2009): 228-47.

“Pharmaceutically acceptable” means that which is useful in preparing a pharmaceutical composition that is generally safe, non-toxic, and neither biologically nor otherwise undesirable, and includes that which is acceptable for veterinary as well as human pharmaceutical use.

Also within the scope of the invention are pharmaceutically acceptable salts of the compounds of the invention. “Pharmaceutically acceptable salts” of a compound means salts that are pharmaceutically acceptable, as defined herein, and that possess the desired pharmacological activity of the parent compound. Such salts include:
(a) acid addition salts formed with inorganic acids such as hydrochloric acid, hydrobromic acid, sulfuric acid, nitric acid, phosphoric acid and the like; or formed with organic acids such as acetic acid, methanesulfonic acid, maleic acid, tartaric acid, citric acid and the like; and
(b) salts formed when an acidic proton present in the parent compound either is replaced by a metal ion, e.g. an alkali metal ion, an alkaline earth ion, or an aluminium ion; or coordinates with an organic or inorganic base. Acceptable organic bases include ethanolamine, diethanolamine, N-methylglucamine, triethanolamine and the like. Acceptable inorganic bases include aluminium hydroxide, calcium hydroxide, potassium hydroxide, sodium carbonate and sodium hydroxide.

The inventors have surprisingly found that dual inhibitors of IDO1 and TDO are useful in treating or preventing refractory cancers.

In one aspect, the present invention provides for the treatment of refractory cancer in a subject, comprising administering to the subject in need thereof a therapeutically effective amount of a dual inhibitor of IDO1 and TDO.

In another aspect, the invention provides for the prevention of the development of refractory cancer, comprising administering to the subject in need thereof a therapeutically effective amount of a dual inhibitor of IDO1 and TDO.

In another aspect, the invention provides for the treatment of a refractory cancer cell, comprising administering a dual inhibitor of IDO1 and TDO to said cancer cell. In various embodiments, the refractory cancer cell is in vitro or in vivo.

In another aspect, the invention provides for the prevention of the development of a refractory cancer cell, the method comprising administering a dual inhibitor of IDO1 and TDO to said cancer cell. In various embodiments, the cancer cell is in vitro or in vivo.

In various embodiments, the refractory cancer or cancer cell is refractory to an anticancer agent and/or a cancer therapy.

### Refractory Cancer

Refractory cancers that may be treated or prevented in accordance with the present invention include, but are not limited to, cancers refractory to anticancer agents. Refractory cancer cells that may be treated or prevented in accordance with the present invention include, but are not limited to, cancer cells refractory to anticancer agents.

Anticancer agents will be apparent to those skilled in the art having regard, for example, to the refractory cancer or cancer cell to be treated or prevented. Numerous anticancer agents are known in the art. Examples of suitable anticancer agents include those listed in Cancer: Principles and Practice of Oncology, 7th Edition, Devita et al, Lippincott Williams & Wilkins, 2005, which is incorporated herein by reference.

In various embodiments, the refractory cancer or cancer cell is refractory to a platinum-based chemotherapeutic agent, for example, a platinum-complex. In various embodiments, the platinum-based chemotherapeutic agent is selected from: carboplatin, cisplatin, lobaplatin, oxaliplatin, picoplatin, nedaplatin, phenanthriplatin and/or satraplatin. For example, in various embodiments, the platinum-based chemotherapeutic agent is selected from cisplatin, carboplatin and/or oxaliplatin.

Accordingly, in various embodiments, the refractory cancer or cancer cell to be treated or prevented is refractory to a platinum-based chemotherapeutic agent selected from: carboplatin, cisplatin, lobaplatin, oxaliplatin, picoplatin, nedaplatin, phenanthriplatin and satraplatin. For example, in various embodiments, the platinum-based chemotherapeutic agent is cisplatin, carboplatin or oxaliplatin.

In other various embodiments, the refractory cancer to be treated or prevented in accordance with the present invention is refractory to an anticancer agent including, but are not limited to, a chemotherapeutic agent selected from the group comprising: alkaloids and natural products, including: camptothecin derivatives for example 9-aminocamptothecin, exatecan, irinotecan rubitecan and topotecan, podophyllum derivatives for example etoposide and teniposide, taxanes for example docetaxel, paclitaxel and paclitaxel poliglumex, vinca alkaloids for example vinblastine, vincristine, vindesine, vinflunine and vinorelbine, and others for example aplidine, elliptinium acetate, irofulven, ixabepilone, kahalalide F, midostaurin and trabectedin; alkylating agents, including alkyl sulfonates for example busulfan, improsulfan and piposulfan, aziridines for example carboquone, diaziquone, mitomycin-C and uredepa, ethylenimines and methylmelamines for example altretamine, triethylenemelamine, triethylenephosphoramide and triethylenethiophosphoramide, nitrogen mustards for example bendamustine, canfosfamide, chlorambucil, chlornaphazine, yclophosamide, cyclophosphamide estramustine, glufosfamide, ifosfamide, mechlorethamine, mechlorethamine oxide hydrochloride, melphalan, perfosfamide, prenimustine, trichlormethine, trofosfamide, and uracil mustard, nitrosoureas for example carmustine, chlorozotocin, fotemustine, lomustine, nimustine and ranimustine, and others for example dacarbazine, etoglucid, mitobronitol, mitolactol, pipobroman, procarbazine, lomustine and temozolomide; antibiotics and analogs, including actinomycins for example cactinomycin and dactinomycin, anthracyclines for example aclacinomycins, amrubicin, carubicin, daunorubicin, doxorubicin, epirubicin, idarubicin, pirarubicin, Valrubicin and zorubicin, and others for example bleomycins, mitomycins, peplomycin, plicamycin, porfiromycin, streptozocin, temsirolimus and zinostatin; antimetabolites, including folic acid analogs and antagonists for example denopterin, edatrexate, methotrexate, nolatrexed, pemetrexed, piritrexi, pteropterin, raltitrexed and trimetrexate, purine analogs for example cladribine, clofarabine, fludarabine, fludarabine phosphate 6-mercaptopurine, nelarabine, thiamiprine, thioguanine and tiazofurine, and pyrimidine analogs for example ancitabine, azacitidine 6-azauridine, capecitabine, carmofur, cytarabine, decitabine, doxifluridine, enocitabine, floxuridine, fluorouracil, gemcitabine, tegafur andtroxacitabine; enzymes for example L-asparaginase and ranpirnase; farnesyl transferase inhibitors for example lonafarnib, tipifarnib; immunomodulators for example aldesleukin, interferon-α, interferon-γ, lentinan, mepact, oregovomab, propagermanium, PSK®, roquinimex, sipuleucel-T, sizofiran, teceleukin and ubenimex; immunotoxins for example cintredekin besudotox and denileukin diftitox; monoclonal antibodies for example alemtuzumab, bevacizumab, cetuximab, edrecolomab, epratuzumab, gemtuzumab, ozogamicin, oregovomab, panitumumab, rituximab, tositumomab¹³¹I, ofatumumab, ipilimumab, pertuzumab, ramucirumab, obinutuzumab, nivolumab, Pembrolizumab, dinutuximab and trastuzumab; antibody drug conjugates for example Ibritumomab tiuxetan, Brentuximab vedotin and Ado-Trastuzumab emtansine; oligonucleotides for example aprinocarsen and oblimersen sodium; retinoids and analogs for example alitretinoin, bexarotene, fenretinidem, mofarotene and tarnibarotene; tyrosine kinase inhibitors for example canertinib, dasatinib, erlotinib, gefitinib, imatinib, lapatinib, sorafenib, sunitinib, axitinib, nilotinib, Pazopanib, Bosutinib, Cabozantinib, Ponatinib, Regorafenib and vatalanib; other inhibitors for example crizotinib, ruxolitinib, vandetanib, Vemurafenib Tofacitinib, Afatinib, Dabrafenib, Ibrutinib and Trametinib; and others for example amsacrine, arsenic trioxide, atrasentan, bisantrene, bortezomib, brostallicin, calcitriol, edotecarin, eflornithine, flavopiridol, gallium nitrate, hydroxyurea, liarozole, lonidamine, miltefosine, mitoguazone, mitoxantrone, nitracrine, pentostatin, perifosine, pixantrone, razoxane, seocalcitol, sobuzoxane, spirogermanium, tirapazamine and vorinostat. Such anticancer agents also include without limitation antineoplastic hormonal agents including androgens for example dromostanolone, epitiostanol, mepitiostane and testolactone; antiadrenals for example aminoglutethimide, mitotane, trilostane; antiandrogens, bicalutainide, flutamide and nilutamide; antiestrogens for example arzoxifene, droloxifene, fulvestrant, idoxifene, tamoxifen and toremifene; antiprogestins for example onapristone; aromatase inhibitors for example aminoglutethimide, anastrozole, exemestane, fadrozole, formestane, letrozole and vorozole; estrogens for example diethylstilbestrol, fosfestrol, hexestrol and polyestradiol phosphate; LH-RH analogs for example abarelix, buserelin, cetrorelix, goserelin, leuprolide and triptorelin; progestogens for example chlormadinone acetate, medroxyprogesterone and megestrol acetate; and somatostatin analogs for example lanreotide. Such anticancer agents also include without limitation antineoplastic photosensitisers for example 6-aminolevulinic acid, methyl aminolevulinate, motexafin lutetium, porfimer sodium, talaporfin and temoporfin; other anti-proliferative agents such as azathioprine, 5-azacytidine cladribine, 2′,2′-difluorodeoxycytidine, erythrohydroxynonyladenine, ethinyl estradiol, 5-fluorodeoxyuridine, 5-fluorodeoxyuridine monophosphate, fluoxymesterone, hydroxyprogesterone caproate, N-phosphonoacetyl-L-aspartate (PALA), semustine, tenipdside, testosterone propionate, thiotepa, trimethylmelamine and uridine. Additional chemotherapeutic agents include but are not limited to compounds listed on the cancer chemotherapy drug regimens in the 14^{th} Edition of the Merck Index (2006), which is hereby incorporated by reference, such as, mesna, prednisolone, prednisone, and raloxifen. For example, in various embodiments, the anticancer agent is a chemotherapeutic agent selected from: alkylating agents, including alkyl sulfonates for example busulfan, improsulfan and piposulfan, aziridines for example carboquone, diaziquone, mitomycin-C and uredepa, ethylenimines and methylmelamines for example altretamine, triethylenemelamine, triethylenephosphoramide and triethylenethiophosphoramide, nitrogen mustards for example bendamustine, canfosfamide, chlorambucil, chlornaphazine, cyclophosamide, estramustine, glufosfamide, ifosfamide, mechlorethamine, mechlorethamine oxide hydrochloride, melphalan, perfosfamide, prenimustine, trichlormethine, trofosfamide, and uracil mustard, nitrosoureas for example carmustine, chlorozotocin, fotemustine, lomustine, nimustine and ranimustine, and others for example dacarbazine, etoglucid, mitobronitol, mitolactol, pipobroman, procarbazine, lomustine and temozolomide; anthracyclines for example aclacinomycins, amrubicin, carubicin, daunorubicin, doxorubicin, epirubicin, idarubicin, pirarubicin, Valrubicin and zorubicin.

In various embodiments, the refractory cancer to be treated or prevented is refractory to a cancer therapy. For example, in various embodiments, a cancer therapy comprising the administration of one or more anticancer agent and/or a radiotherapy.

In various embodiments, the cancer therapy comprises the administration of one or more anticancer agent, for example, an anticancer agent selected from: a chemotherapeutic agent and/or a radiotherapeutic agent. For example, in various embodiments, a cancer therapy comprising the administration of one or more anticancer agent and wherein the anticancer agent is selected from those referenced in the discussion above in the context of cancers refractory to an anticancer agent.

In various embodiments, the cancer therapy is a platinum-based chemotherapy comprising the administration of one or more platinum-based chemotherapeutic agent, optionally in combination with one or more other anticancer agent. For example, in various embodiments, the platinum-based chemotherapy may comprise a step of administering one or more platinum-based chemotherapeutic agent selected from carboplatin, cisplatin, lobaplatin, oxaliplatin, picoplatin, nedaplatin, phenanthriplatin and satraplatin. In various embodiments, the platinum-based chemotherapy comprises a step of administering carboplatin, cisplatin and/or oxaliplatin.

In various embodiments, the cancer therapy is a radiotherapy. Accordingly, in various embodiments, the refractory cancer or cancer cell to be treated or prevented is refractory to a radiotherapy.

In various embodiments, the radiotherapy is external radiation treatment or internal radiation treatment.

Without being bound to theory, reactive oxygen species (ROS) can increase after exposure to physical agents such as ultraviolet rays and heat, and after chemotherapy and radiotherapy. There are multiple mechanisms of increasing ROS, such as by a direct generation of ROS or interfering with the antioxidant system.

Without being bound by theory, prolonged treatment of cancer with chemotherapeutic anticancer agents can induce ROS accumulation. One mechanism by which chemotherapy-resistant cells tolerate high ROS levels is by activation of the kynurenine (KYN) pathway during excessive oxidative stress, to mitigate ROS-induced damage. Once again, without being bound by theory, high ROS levels are a feature found in cisplatin resistant (CR) lung cancer cells.

Without being bound by theory, one mechanism by which the dual inhibitors of IDO1 and TDO of the present invention may exert a therapeutic effect is by causing further ROS accumulation in refractory cancer cells and causing ROS-induced cell death in said cells.

In various embodiments, the mechanism of resistance of the refractory cancer or cancer cell to be treated or prevented is resistance to ROS-induced cell death, for example, ROS-dependent programmed cell death (PCD).

Accordingly, the methods and uses described herein may be useful in treating or preventing refractory cancer, wherein the refractory cancer is refractory to an anticancer agent and/or cancer therapy that induces ROS accumulation within cancer cells, for example, an anticancer agent and/or cancer therapy that induces ROS-dependent programmed cell death (PCD) in cancer cells. In various embodiments, the anticancer agent and/or cancer therapy is characterized by inducing ROS accumulation within cancer cells and the refractory cancer is characterized by being resistant to said ROS accumulation.

A wide range of refractory cancers or cancer cells may be treated or prevented by the dual inhibitor of IDO1 and TDO of the present invention. Refractory cancers, or cells thereof, that may be treated or prevented in accordance with the present invention include, but are not limited to: colorectal cancer, breast cancer, melanoma, reproductive organ cancer, respiratory tract cancer, brain cancer, digestive tract cancer, urinary tract cancer, eye cancer, liver cancer, skin cancer, head and neck cancer, thyroid cancer, parathyroid cancer and/or their distant metastases. Those disorders also include lymphomas, sarcomas, and leukemias.

Examples of refractory breast cancer include, but are not limited to invasive ductal carcinoma, invasive lobular carcinoma, ductal carcinoma in situ, and lobular carcinoma in situ.

Examples of refractory cancers of the respiratory tract include, but are not limited to small-cell and non-small-cell lung carcinoma, as well as bronchial adenoma and pleuropulmonary blastoma.

Examples of refractory brain cancers include, but are not limited to glioblastoma, brain stem and hypophthalmic glioma, cerebellar and cerebral astrocytoma, medulloblastoma, ependymoma, as well as neuroectodermal and pineal tumor.

Tumors of the refractory male reproductive organs include, but are not limited to, prostate and testicular cancer.

Tumors of the refractory female reproductive organs include, but are not limited to endometrial, cervical, ovarian, ovarian adenocarcinoma, vaginal, and vulvar cancer, as well as sarcoma of the uterus.

Tumors of the refractory digestive tract include, but are not limited to anal, colon, colorectal, esophageal, gallblader, gastric, pancreatic, rectal, small-intestine, and salivary gland cancers.

Tumors of the refractory urinary tract include, but are not limited to bladder, penile, kidney, renal pelvis, ureter, and urethral cancers.

Refractory eye cancers include, but are not limited to, intraocular melanoma and retinoblastoma.

Examples of refractory liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Refractory skin cancers include, but are not limited to squamous cell carcinoma, Kaposi's sarcoma, malignant melanoma, Merkel cell skin cancer, and non-melanoma skin cancer.

Refractory head-and-neck cancers include, but are not limited to, laryngeal/hypopharyngeal/nasopharyngeal/oropharyngeal cancer, and lip and oral cavity cancer. Lymphomas include, but are not limited to AIDS-related lymphoma, non-Hodgkin's lymphoma, cutaneous T-cell lymphoma, Hodgkin's disease, and lymphoma of the central nervous system.

Refractory sarcomas include, but are not limited to, sarcoma of the soft tissue, osteosarcoma, malignant fibrous histiocytoma, lymphosarcoma, and rhabdomyosarcoma. Leukemias include, but are not limited to acute myeloid leukemia, acute lymphoblastic leukemia, chronic lymphocytic leukemia, chronic myelogenous leukemia, and hairy cell leukemia.

The refractory cancer may be a solid tumor or a liquid tumor. In various embodiments, the refractory cancer is a solid tumor.

These disorders have been well characterized in humans, but also exist with a similar etiology in other warm-blooded mammals, and can be treated by the compounds of the present invention.

Accordingly, in various embodiments, the invention provides methods of treating or preventing a refractory cancer, or cells thereof, wherein the cancer is selected from one or more of the cancers listed above and is refractory to an anticancer agent and/or cancer therapy.

In various embodiment, the refractory cancer or refractory cancer cell is a refractory breast cancer, refractory cancer of the respiratory tract, refractory brain cancer, refractory tumor of the male reproductive organs, refractory tumor of the female reproductive organs, refractory tumor of the digestive tract, refractory tumors of the urinary tract, refractory eye cancer, refractory liver cancer, refractory skin cancer, refractory head-and-neck cancer, refractory Lymphoma, refractory sarcoma or refractory leukemia, for example, those referenced above. In various embodiments, the cancer is a solid tumor. In another embodiment, the cancer is a liquid tumor.

In various embodiments, the invention provides methods of treating or preventing refractory lung cancer, refractory pancreatic cancer, refractory ovarian cancer or refractory breast cancer.

In various embodiments, in the method of treating refractory cancer or method of treating a refractory cancer cell, the subject or cancer cell has previously been administered an anticancer agent, and the refractory cancer or cancer cell is refractory to said anticancer agent.

The dual inhibitors of IDO1 and TDO described herein are also useful in preventing the development of refractory cancer or a refractory cancer cell. For example, in various embodiments, preventing the development of refractory cancer or a refractory cancer cell from a cancer or cancer cell that is not refractory to said anticancer agent and/or therapy.

In various embodiments, the methods of preventing the development of cancer or a cancer cell refractory to an anticancer agent and/or cancer therapy, comprise administering the dual inhibitor of IDO1 and TDO in combination with the anticancer agent and/or cancer therapy. The dual inhibitor and the anticancer agent and/or cancer therapy may be administered simultaneously, sequentially, or separately and may be administered as a single formulation or as separate formulations, as appropriate.

In various embodiments, the methods of preventing the development of refractory cancer or refractory cancer cell further comprise administering one or more additional anticancer agent and/or cancer therapy, in combination with the dual inhibitor of IDO1 and TDO and the anticancer agent and/or cancer therapy which the cancer or cancer cell is to be prevented from becoming refractory to. The dual inhibitor, the anticancer agent and/or cancer therapy, and the one or more additional anticancer agent and/or cancer therapy may be administered simultaneously, sequentially, or separately and may be administered as a single formulation or as separate formulations, as appropriate.

For example, in various embodiments, the invention provides a method of preventing the development of cancer or a cancer cell refractory to a platinum-based chemotherapeutic agent, comprising administering the dual inhibitor of IDO1 and TDO in combination with the platinum-based chemotherapeutic agent.

In another aspect, the invention provides methods of increasing the efficacy of an anticancer agent and/or cancer therapy, comprising administering to a subject with cancer a therapeutically effective amount of (a) the dual inhibitor of IDO1 and TDO and (b) the anticancer agent and/or therapy.

In another aspect, the invention provides methods of increasing the sensitivity of a refractory cancer or refractory cancer cell to an anticancer agent and/or cancer therapy, comprising administering to a subject or refractory cancer cell an effective amount of (a) the dual inhibitor of IDO1 and TDO and (b) the anticancer agent and/or therapy.

The dual inhibitors of IDO1 and TDO described herein are also expected to be useful in preventing the development of refractory cancer in a subject having cancer at risk of becoming refractory to an anticancer agent and/or cancer therapy. Accordingly, in some embodiments, the invention provides a method of preventing the development of refractory cancer in a subject having cancer at risk of becoming refractory to an anticancer agent and/or cancer therapy, comprising administering to the subject a therapeutically effective amount of (a) the dual inhibitor of IDO1 and TDO and (b) the anticancer agent and/or cancer therapy.

A cancer or a cancer cell at risk of becoming refractory to an anticancer agent and/or cancer therapy includes cancer or cancer cells where there is a significant risk or probability that the cancer or cancer cell will become refractory to the anticancer agent and/or cancer therapy. For example, it will be appreciated that in certain cancers treated with platinum based chemotherapeutic agents there is a significant risk of the cancer becoming refractory to said platinum based chemotherapeutic agent. In such situations, it would be advantageous to administer a dual inhibitor of IDO1 and TDO of the present invention in combination with the platinum based chemotherapeutic agent to prevent development of the refractory cancer.

The methods of preventing the development of refractory cancer or a refractory cancer cell described herein thus provide for or enable the treatment of cancers or cancer cells at risk of becoming refractory to the anticancer agent and/or therapy with the anticancer agent. In various embodiments, the methods of preventing the development of refractory cancer further comprise treating the cancer or cancer cell at risk of becoming refractory to the anticancer agent and/or therapy.

For example, in various embodiments, the method is for preventing the development of refractory cancer in a subject having cancer at risk of becoming refractory to a platinum based chemotherapeutic agent and comprises administering to the individual a therapeutically effective amount of (a) the dual inhibitor of IDO1 and TDO and (b) the platinum based chemotherapeutic agent.

In various embodiments, the method of preventing the development of refractory cancer comprises administering to the subject or cancer cell, the dual inhibitor of IDO1 and TDO in combination with an anticancer agent and/or cancer therapy, wherein the respective amounts of the dual inhibitor of IDO1 and TDO and the anticancer agent and/or cancer therapy are effective to prevent or delay the development of cell resistance to the anticancer agent and/or cancer therapy.

In various embodiments, the methods of treating refractory cancer or a refractory cancer cell that is refractory to an anticancer agent and/or cancer therapy, comprise administering the dual inhibitor of IDO1 and TDO in combination with the anticancer agent and/or cancer therapy. The dual inhibitor and the anticancer agent and/or cancer therapy may be administered simultaneously, sequentially, or separately and may be administered as a single formulation or as separate formulations, as appropriate.

In various embodiments, the anticancer agent and/or cancer therapy administered in combination with the dual inhibitor of IDO1 and TDO is an anticancer agent and/or cancer therapy as referenced in the discussion above in the context of cancers refractory to the anticancer agent and/or cancer therapy.

In various embodiments, the dual inhibitor of IDO1 and TDO and the anticancer agent and/or cancer therapy can optionally be administered in combination with one or more additional anticancer agent and/or cancer therapy.

The dual inhibitors of IDO1 and TDO described herein are also expected to be useful in treating refractory cancer either alone, or in combination with one or more additional anticancer agent and/or cancer therapy. Accordingly, in various embodiments, the method of treating refractory cancer or refractory cancer cell, comprises administering the dual inhibitor of IDO1 and TDO in combination with one or more additional anticancer agent and/or cancer therapy. The dual inhibitor and the one or more additional anticancer agent and/or cancer therapy may be administered simultaneously, sequentially, or separately and may be administered as a single formulation or as separate formulations, as appropriate.

In various embodiments, the one or more additional anticancer agent and/or cancer therapy is, for example, an anticancer agent and/or cancer therapy as referenced in the discussion above in the context of refractory cancers. For example, in various embodiments, the one or more additional anticancer agent may be a platinum based chemotherapeutic agent, such as a platinum complex. For example, in various embodiments, the platinum based chemotherapeutic agent is selected from: carboplatin, cisplatin, lobaplatin, oxaliplatin, picoplatin, nedaplatin, phenanthriplatin and/or satraplatin. In a particular example, the platinum based chemotherapeutic agent is selected from: cisplatin, carboplatin and/or oxaliplatin.

The dual inhibitor of IDO1 and TDO can also be administered in combination with a radiotherapy or radiotherapeutic agent.

In various embodiments, the one or more additional anticancer agent and/or cancer therapy is selected from the group consisting of: chemotherapeutic agents, immune-modulating agents such as anticancer vaccines, modulators of immune checkpoint proteins, adoptive T cell immunotherapies (for example chimeric antigen receptor T cells (CART cells)), and radiotherapy.

Suitable one or more additional anticancer agents will be apparent to those skilled in the art having regard, for example, to the cancer to be treated. Numerous anticancer agents are known in the art. Examples of suitable anticancer agents include those listed in Cancer: Principles and Practice of Oncology, 7^{th} Edition, Devita et al, Lippincott Williams & Wilkins, 2005, which is incorporated herein by reference.

In various embodiments, the one or more additional anticancer agent induces an increase in ROS within cancer cells. In various embodiments, the one or more additional anticancer agent induces reactive oxygen species (ROS) dependent programmed cell death (PCD) in cancer cells.

In various embodiments, the one or more additional anticancer agent is a chemotherapeutic agents selected from, but are not limited to, compounds listed on the cancer chemotherapy drug regimens in the 14^{th} Edition of the Merck Index (2006), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycin), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

In various embodiments, the one or more additional anticancer agent is a anti-proliferative agents, selected from, but are not limited to, BCNU, CCNU, DTIC, and actinomycin D. Still further anti-proliferative agents include but are not limited to those compounds acknowledged to be used in the treatment of neoplastic diseases in Goodman and Gilman's The Pharmacological Basis of Therapeutics (Eleventh Edition), editor Molinoff et al., publ. by McGraw-Hill, pages 1225-1287 (2006), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyladenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, tenipdside, testosterone propionate, thiotepa, trimethylmelamine, uridine, and vinorelbine.

In various embodiments, the one or more additional anticancer agent is an anti-proliferative agents selected from but are not limited to, other molecular targeted agents, which block the growth of cancer cells by interfering with specific targeted molecules needed for carcinogenesis and tumour growth. Examples include small molecule protein and lipid kinase inhibitors, monoclonal antibodies, molecularly targeted humanised monoclonal antibodies and monoclonal antibody drug conjugates. Examples of such inhibitors include: Rituximab, Trastuzumab, Alemtuzumab, Tositumomab-I131, Cetuximab, Ibritumomab tiuxetan, Bevacizumab, Panitumumab, Ofatumumab, Ipilimumab, Brentuximab vedotin, Pertuzumab, Ado-Trastuzumab emtansine, Ramucirumab, Obinutuzumab, Nivolumab, Pembrolizumab, Dinutuximab, Imatinib, Gefitinib, Erlotinib, Sorafenib, Dasatinib, Sunitinib, Lapatinib, Nilotinib, Pazopanib, Crizotinib, Ruxolitinib, Vandetanib, Vemurafenib, Axitinib, Bosutinib, Cabozantinib, Ponatinib, Regorafenib, Tofacitinib, Afatinib, Dabrafenib, Ibrutinib and Trametinib.

In various embodiments, the one or more additional anticancer agent is an immune modulating agents. For example, in various embodiments, the immune modulating agent is selected from, without limitation, anticancer vaccines, agents that modulate immune checkpoint proteins (such as CTLA4, PD1-4s and PD-L1) and adoptive T-cell therapies (such as CARTs). In various embodiments, the one or more additional anticancer agent is an inhibitor of CTLA4, an anti-PD-1 antibody or an anti-PD-L1 antibody. For example, in various embodiments, the one or more additional anticancer agent is an inhibitor of CTLA4 such as Ipilimumab, an anti-PD-1 antibody such as Cemiplimab, Nivolumab and/or Pembrolizumab, or an anti-PD-L1 antibody such as Atezolizumab, Avelumab and/or Durvalumab.

The dual inhibitor of IDO1 and TDO may be administered simultaneously, sequentially, or separately with the one or more additional anticancer agents, therapy and/or an immune modulating agent. The dual inhibitor of IDO1 and TDO and the one or more additional anticancer agents and/or an immune modulating agent may be administered as single formulation or as separate formulations.

Accordingly, the dual inhibitor of IDO1 and TDO can be administered either alone or in combination with one or more other such anticancer agents or therapies, either simultaneously or sequentially dependent upon the particular cancer to be treated.

In various embodiments, the one or more additional cancer therapy comprises a step of administering the one or more additional anticancer agent. In various embodiments, the one or more additional cancer therapy is a radiotherapy.

In various embodiments, the one or more additional cancer therapy is an immunotherapy selected from Ipilimumab (an inhibitor of CTLA4), Nivolumab and Pembrolizumab (both inhibitors of PD-1).

Dual inhibitors of IDO1 and TDO

In broad terms the invention relates to the use of dual inhibitors of IDO1 and TDO for treating or preventing refractory cancer.

The term “dual inhibitor of IDO1 and TDO”, and similar terms such as “dual inhibitors of IDO1 and TDO”, means a compound that inhibits the enzyme activity of IDO1 and inhibits the enzyme activity of TDO, in particular, a compound that inhibits the cellular enzyme activity of IDO1 and inhibits the cellular enzyme activity of TDO, for example, a compound that has a cellular IDO1 IC₅₀ of less than 100 M and a cellular TDO IC₅₀ of less than 100 M.

Unless indicated otherwise, the inhibition of cellular enzyme activity of IDO1 and the inhibition of cellular enzyme activity of TDO are each as determined by the methods described in the examples herein.

In various embodiments, the dual inhibitor of IDO1 and TDO described herein has an cellular IDO1 IC₅₀ of less than 100 μM, less than 90 μM, less than 80 μM, less than 70 μM, less than 60 μM, less than 50 μM, less than 40 μM, less than 30 μM, less than 20 μM, less than 15 μM, less than 10 M or less than 1 M as determined by the Cell-based assay for IDO1 inhibition described in the examples; and has a cellular TDO IC₅₀ of less than 100 μM, less than 90 μM, less than 80 μM, less than 70 μM, less than 60 μM, less than 50 μM, less than 40 μM, less than 30 μM, less than 20 μM, less than 15 μM, less than 10 M or less than 1 M as determined by the Cell-based assay for TDO inhibition described in the examples. In various embodiments, the dual inhibitor of IDO1 and TDO described herein has a cellular IDO1 IC₅₀ of less 10 M as determined by the Cell-based assay for IDO1 inhibition described in the examples; and has a cellular TDO IC₅₀ of less than 10 M as determined by the Cell-based assay for TDO inhibition described in the examples.

In various embodiments, the dual inhibitor of IDO1 and TDO described herein has a cellular IDO1 IC₅₀ of less than 10 μM, less than 9 μM, less than 8 μM, less than 7 μM, less than 6 μM, less than 5 μM, less than 4 μM, less than 3 μM, less than 2 μM, less than 1 μM, less than 0.9 μM, less than 0.8 μM, less than 0.7 μM, less than 0.6 μM, less than 0.5 μM, less than 0.4 μM, less than 0.3 μM, less than 0.2 M and/or less than 0.1 M as determined by the Cell-based assay for IDO1 inhibition described in the examples; and has a cellular TDO IC₅₀ of less than 10 μM, less than 9 μM, less than 8 μM, less than 7 μM, less than 6 μM, less than 5 μM, less than 4 μM, less than 3 μM, less than 2 μM, less than 1 μM, less than 0.9 μM, less than 0.8 μM, less than 0.7 μM, less than 0.6 μM, less than 0.5 μM, less than 0.4 μM, less than 0.3 μM, less than 0.2 M and/or less than 0.1 M as determined by the Cell-based assay for TDO inhibition described in the examples. In various embodiments, the dual inhibitor of IDO1 and TDO described herein has a cellular IDO1 IC₅₀ of less 1 M as determined by the Cell-based assay for IDO1 inhibition described in the examples; and has a cellular TDO ICD of less than 1 M as determined by the Cell-based assay for TDO inhibition described in the examples.

In various embodiments of the invention, the dual inhibitor of IDO1 and TDO described herein has a cellular IDO1 IC₅₀ of less than 1 μM, less than 0.9 μM, less than 0.8 μM, less than 0.7 μM, less than 0.6 μM, less than 0.5 μM, less than 0.4 μM, less than 0.3 μM, less than 0.2 M and/or less than 0.1 M as determined by the Cell-based assay for IDO1 inhibition described in the examples herein; and has a cellular TDO IC₅₀ of less than 1 μM, less than 0.9 μM, less than 0.8 μM, less than 0.7 μM, less than 0.6 μM, less than 0.5 μM, less than 0.4 μM, less than 0.3 μM, less than 0.2 M and/or less than 0.1 M as determined by the Cell-based assay for TDO inhibition described in the examples herein.

In various embodiments, the dual inhibitor of IDO1 and TDO described herein has cellular TDO inhibitory activity that is greater than, or equal to, the IDO1 inhibitory activity. In a particular embodiment, the dual inhibitor of IDO1 and TDO has a cellular IDO1 IC₅₀ that is higher or equal to the cellular TDO IC₅₀.

In various embodiments, the dual inhibitor of IDO1 and TDO is a 3-aminoisosoxazole, preferably a 3-aminoisosoxazole fused to a 6 membered aromatic ring comprising at least one ring nitrogen atom, such as certain compounds of Formula I as described herein.

As described in detail in PCT/IB2015/056129, which is incorporated herein by reference, compounds of Formula I have been found to be inhibitors of indoleamine 2,3-dioxygenase 1 (IDO1) and/or tryptophan2,3-dioxygenase (TDO).

In various embodiments, the dual inhibitor of IDO1 and TDO for use in the present invention is a compound of the general Formula I with IDO1 and TDO inhibitory activity.

The compounds as defined in paragraphs (1) to (49) of the summary of invention section and that have dual IDO1 and TDO inhibitory activity, may be used in any of the methods, uses and other aspects of the invention described herein. Accordingly, in various embodiments of the invention, the dual inhibitor of IDO1 and TDO is a compound as defined in paragraphs (1) to (49), and that has inhibitory IDO1 and TDO activity.

The dual inhibitors of IDO1 and TDO as defined in paragraphs (46) to (49) in the summary of invention section, may be used in any of the methods, uses and other aspects of the invention described herein. Accordingly, in various embodiments, the dual inhibitor of IDO1 and TDO is a compound as defined in paragraphs (46) to (49).

In various embodiments, the dual inhibitor of IDO1 and TDO is a compound as defined in paragraphs (48) to (49).

In various embodiments, the dual inhibitor of IDO1 and TDO is 6-Chloro-5-fluoroisoxazolo[5,4-b]pyridin-3-amine (also referred to herein as compound (66) or AT-0174).

Compounds of Formula I may be prepared by following the methods described in PCT International patent application number PCT/IB2015/056129 (published as WO2016024233), which is incorporated herein by reference, or procedures analogous thereto. The person skilled in the art would appreciate that other dual inhibitor of IDO1 and TDO are also suitable for use in the invention.

Other inhibitors of IDO1 and/or TDO and methods for their preparation are described in PCT International patent application number PCT/NZ2016/050135 (published as WO 2017034420), which is incorporated herein by reference.

Compounds as described in PCT/NZ2016/050135 that are dual inhibitors of IDO1 and TDO are suitable for use in the present invention. For example, PCT/NZ2016/050135 discloses the following compounds:
1-(5-Chloro-4,6-dimethylisoxazolo[5,4-b]pyridin-3-yl)-3-(4-(trifluoromethoxy)phenyl)urea (2B)
1-(5-Chloro-4,6-dimethylisoxazolo[5,4-b]pyridin-3-yl)-3-(2-chlorophenyl)urea (6B)
1-(5-Chloro-4,6-dimethylisoxazolo[5,4-b]pyridin-3-yl)-3-(3-chlorophenyl)urea (7B)
1-(5-Chloro-4,6-dimethylisoxazolo[5,4-b]pyridin-3-yl)-3-(4-chlorophenyl)urea (8B)
1-(5-Chloro-4,6-dimethylisoxazolo[5,4-b]pyridin-3-yl)-3-(2-(trifluoromethoxy)phenyl)urea (9B)
1-(5-Chloro-4,6-dimethylisoxazolo[5,4-b]pyridin-3-yl)-3-(3-(trifluoromethoxy)phenyl)urea (10B)
1-(5-Chloro-4,6-dimethylisoxazolo[5,4-b]pyridin-3-yl)-3-(3-methoxyphenyl)urea (12B)
N-(5-Chloro-4,6-dimethylisoxazolo[5,4-b]pyridin-3-yl)-2-phenylacetamide (18B)
N-(5-Chloro-4,6-dimethylisoxazolo[5,4-b]pyridin-3-yl)-2-(4-(trifluoromethoxy)phenyl)acetamide (19B)
Phenyl (5-chloro-4,6-dimethylisoxazolo[5,4-b]pyridin-3-yl)carbamate (4B)
1-(5-Chloro-4,6-dimethylisoxazolo[5,4-b]pyridin-3-yl)-3-(3-(trifluoromethyl)phenyl)urea (15B)
4-Fluorophenyl (5-chloro-4,6-dimethylisoxazolo[5,4-b]pyridin-3-yl)carbamate (5B)
N-(5-Chloro-4,6-dimethylisoxazolo[5,4-b]pyridin-3-yl)acetamide (20B),

and a pharmaceutically acceptance salts thereof.

Dual inhibitors of IDO1 and TDO suitable for use in the invention include Phenyl (5-chloro-4,6-dimethylisoxazolo[5,4-b]pyridin-3-yl)carbamate (4B) and 1-(5-Chloro-4,6-dimethylisoxazolo[5,4-b]pyridin-3-yl)-3-(3-(trifluoromethyl)phenyl)urea (15B).

### Administration

It will be appreciated by those skilled in the art that a particular method of therapy for administering the dual inhibitors of IDO1 and TDO, as described herein, will employ a selected route of administration which will in turn depend on a variety of factors, all of which are considered routinely when administering therapeutics. It will be further appreciated by one skilled in the art that the optimal course of treatment or prevention, i.e., the mode of treatment and the daily number of doses of a compound of this invention given for a defined number of days, can be ascertained by those skilled in the art using conventional treatment tests.

Therapeutic dosages of the dual inhibitors of IDO1 and TDO, as described herein, will likely be in the range of 1 mg to 2000 mg per day.

In various embodiments, the dose of the dual inhibitor of IDO1 and TDO is in the range of 1 to 100 mg per day, 100 to 200 mg per day, 200 to 300 mg per day, 300 to 400 mg per day, 400 to 500 mg per day, 500 to 600 mg per day, 600 to 700 mg per day, 700 to 800 mg per day, 800 to 900 mg per day, 900 to 1000 mg per day. In various embodiments, the dose of the dual inhibitor of IDO1 and TDO is in the range of 1000 to 1100 mg per day, 1100 to 1200 mg per day, 1200 to 1300 mg per day, 1300 to 1400 mg per day, 1400 to 1500 mg per day, 1500 to 1600 mg per day, 1600 to 1700 mg per day, 1700 to 1800 mg per day, 1800 to 1900 mg per day or 1900 to 2000 mg per day.

In various embodiments, the dose of the dual inhibitor of IDO1 and TDO is in the range of 1 mg to 50 mg per day, 50 mg to 100 mg per day, 201 mg to 250 mg per day, 250 mg to 300 mg per day, 300 mg to 350 mg per day, 350 mg to 400 mg per day, 400 mg to 450 mg per day, 450 mg to 500 mg per day, 500 mg to 550 mg per day, 550 mg to 600 mg per day, 600 mg to 650 mg per day, 650 mg to 700 mg per day, 700 mg to 750 mg per day, 750 mg to 800 mg per day, 800 mg to 850 mg per day, 850 mg to 900 mg per day, 900 mg to 950 mg per day, 950 mg to 1000 mg per day.

The specific dose level selected for any particular patient will depend upon a variety of factors, including the activity of the specific compound employed, the age, body weight, general health, sex, diet, time of administration, route of administration, and rate of excretion, drug combination and the severity of the condition undergoing therapy.

Dosage levels of the active ingredients in the pharmaceutical compositions may be varied so as to provide an amount of the active ingredient which is effective to achieve the desired therapeutic effect for a particular patient, composition, and mode of administration, without being toxic to the patient (an effective amount).

### Pharmaceutical Compositions

The dual inhibitor of indoleamine-2,3-dioxygenase (IDO1) and tryptophan-2,3-dioxygenase (TDO), may be formulated or provided for use as a pharmaceutical composition comprising the dual inhibitor of IDO1 and TDO, and a pharmaceutically acceptable carrier.

The term “pharmaceutically acceptable carrier” refers to a carrier (e.g. adjuvant, buffer, stabiliser or vehicle) that may be administered to a subject together with the dual inhibitor of IDO1 and TDO, which is generally safe. The pharmaceutically acceptable excipient, adjuvant, carrier, buffer or stabiliser should be nontoxic and should not interfere with the efficacy of the active ingredient. The term “pharmaceutically acceptable carrier” also including carriers suitable in veterinary as well as human pharmaceutical use. The precise nature of the carrier or other material will depend on the route of administration.

The dual inhibitor of IDO1 and TDO may be administered orally, topically, parenterally, by inhalation or spray or rectally in dosage unit formulations. The term ‘administration by injection’ includes intravenous, intramuscular, subcutaneous and parenteral injections, as well as use of infusion techniques. One or more compounds may be present in association with one or more non-toxic pharmaceutically acceptable carriers and if desired other active ingredients.

Compositions intended for oral use may be prepared according to any suitable method known to the art for the manufacture of pharmaceutical compositions. Such compositions may contain one or more agents selected from the group consisting of diluents, sweetening agents, flavoring agents, coloring agents and preserving agents in order to provide palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; and binding agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed. These compounds may also be prepared in solid, rapidly released form.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropyl methylcellulose, sodium alginate, polyvinylpyrrolidone, gum tragacanth and gum acacia; dispersing or wetting agents may be a naturally occurring phosphatide, for example, lecithin, or condensation products or an alkylene oxide with fatty acids, for example polyoxyethylene stearate, or condensation products of ethylene oxide with long chain aliphatic alcohols, for example heptadecaethylene oxycetanol, or condensation products of ethylene oxide with partial esters derived from fatty acids and hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, flavoring and coloring agents, may also be present.

The compounds may also be in the form of non-aqueous liquid formulations, e.g., oily suspensions which may be formulated by suspending the active ingredients in a vegetable oil, for example arachis oil, olive oil, sesame oil or peanut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents.

The compounds may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter and polyethylene glycols.

### Kits

The dual inhibitor of indoleamine-2,3-dioxygenase (IDO1) and tryptophan-2,3-dioxygenase (TDO) may be formulated or provided for use as a kit comprising one or more dual inhibitor of IDO1 and TDO; and optionally one or more additional therapeutic agent; and instructions for using the dual inhibitor in the methods described. In various embodiments, the therapeutic agent is an anticancer agent as described hereinabove.

The dual inhibitor of IDO1 and TDO is typically in the form of a pharmaceutical composition, and contained within a container. The container may be any vessel or other sealed or sealable apparatus that can hold the pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form and is made of a pharmaceutically acceptable material, for example a paper or cardboard box, a glass or plastic bottle or jar, a re-sealable bag, or a blister pack with individual doses for pressing out of the pack according to a therapeutic schedule. The container employed typically depends on the dosage form involved. More than one container can be used together in a single package for a single dosage form.

The kits may also comprise a device to administer or to measure out a unit dose of the pharmaceutical composition. The device may include, for example, an inhaler if the composition is an inhalable composition; a syringe and needle if the composition is an injectable composition; a syringe, spoon, pump, or a vessel with or without volume markings if the composition is an oral liquid composition; or any other measuring or delivery device appropriate to the dosage formulation of the composition present in the kit.

In various embodiments, the kits may comprise, for example in a separate vessel or container, one or more additional therapeutic agent, typically in the form of a pharmaceutical composition comprising the additional therapeutic agent and a pharmaceutically acceptable carrier. The additional therapeutic agent may be selected from any of those indicated herein for co-administration with a dual inhibitor of IDO1 and TDO as described herein.

The above methods should be considered in no way limiting and suitable variations or alternatives will be apparent to those skilled in the art.

### EXAMPLES

The following non-limiting examples are provided to illustrate the present invention and in no way limit the scope thereof.

### Example 1

### Enzymatic Assay for IDO1 Activity

Recombinant human IDO1 (rhIDO1) was expressed and purified from cultures of EC538 strain of E. coli transformed with pREP4 and pQE9-IDO plasmid. Reaction mixes were set up in 384-well microplates containing 50 mM phosphate buffer, 10 mM ascorbic, 10 μM methylene blue, 100 μg/mL catalase, 80 μM TRP, 0.01% Tween 20 (v/v) mixed with rhIDO1 (15 μL) at a final concentration of 9 nM in a total volume of 30 μL assay medium. The plates were incubated at 37° C. for 30 min, and the enzymatic reaction was terminated by adding piperidine (200 mM) and heated at 65° C. for 20 min. Fluorescence intensity was read at λₑₓ 400 nm and λₑₘ 500 nm. Test compounds were dissolved in 100% DMSO and pre-diluted in assay medium prior to adding rhIDO1. IDO1 inhibition (%) was calculated as

$$( \frac{( \vert uninhibited enzyme assay signal \vert - \vert inhibited enzyme signal \vert )}{( \vert uninhibited enzyme assay signal \vert - \vert assay medium signal \vert )} \times 100 )$$

All experiments were carried out in triplicates, and statistical Analyses were conducted in Prism v5 (Graphpad Software, Inc., La Jolla, CA, USA).

### Cell-Based Assay for IDO1 Inhibition

For the assay of inhibition of cellular IDO1 activity, Lewis Lung carcinoma cells transfected to express human IDO1 (LLTC-hIDO1) or murine (LLTC-mIDO1) were cultured with test compounds at 37° C., 5% CO₂ for 24 h. Culture supernatant from each well was then transferred into a fresh, flat-bottomed 96-well plate, mixed with trichloroacetic acid (10% final concentration) and incubated for 20 min at 60° C. Plates were then centrifuged (10 min at 2500 g) and the supernatants were then transferred and mixed 1:1 with 4-(dimethylamino)benzaldehyde (20 mg/mL in acetic acid) in a new plate. The absorbance of each well was read at 480 nm, and the concentration that inhibited 50% cellular enzyme activity was calculated.

The viability of the cells in each well in the same experiment was determined using the 3-(4,5-dimethylthiazolyl-2-yl)-2,5-diphenyltetrazolium bromide (MTT) colourimetric assay. After the removal of the supernatant for determination of IDO1 inhibition, the cells were incubated with MTT (500 μg/mL) until crystal formation was observed. Plates were centrifuged for 15 min at 2500 g, and then all the supernatant in then wells was discarded. DMSO (100 μL/well) was added to dissolve the crystals and then the absorbance in each well was measured at 570 nm. Cell viability in each well was expressed as a percentage of untreated controls. Triplicate cultures were used for all experiments unless stated otherwise.

### Cell-Based Assay for TDO Inhibition

For the assay of inhibition of cellular TDO, GL261 cells transfected to over-express full length human TDO were cultured with test compounds at 37° C., 5% CO₂ for 24 h. Culture supernatant from each well was then transferred into a fresh, flat-bottomed 96-well plate, and kynurenine content was determined as described above for the IDO1 assay, and the concentration that inhibited 50% cellular enzyme activity was calculated.

The viability of the cells in each well in the same experiment was determined using the 3-(4,5-dimethylthiazolyl-2-yl)-2,5-diphenyltetrazolium bromide (MTT) colourimetric assay.

Results of the assays are shown in the table 1 below.

### Compound Activity

TABLE 1

| | | Isolated | | | |
|---|---|---|---|---|---|
| | | IDO1 | | | |
| | | Enayme | Cellular | Cellular | |
| | | Assay | IDO1 | TDO | |
| | | IC₅₀ | IC₅₀ | IC₅₀ | Cytotoxicity |
| Compound | Structure | (μM) | (μM) | (μM) | IC₅₀ (μM) |
| 66 | 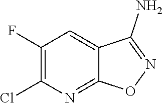 [SMILES: Nc1noc2nc(Cl)c(F)cc12] | A | A | A | D |
| 6 | 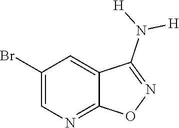 [SMILES: Nc1noc2ncc(Br)cc12] | C | A | A | D |
| 8 | 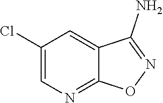 [SMILES: Nc1noc2ncc(Cl)cc12] | C | A | A | D |
| 11 | 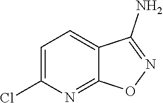 [SMILES: Nc1noc2nc(Cl)ccc12] | B | A | A | D |
| 16 | 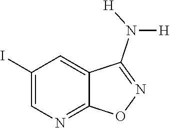 [SMILES: Nc1noc2ncc(I)cc12] | B | A | A | D |
| 49 | 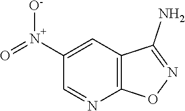 | B | A | A | D |
| 67 | 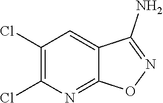 [SMILES: Nc1noc2nc(Cl)c(Cl)cc12] | B | B | A | D |
| 3 | 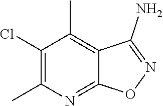 | B | A | C | C |
| 4 | 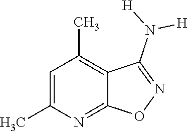 | C | B | C | D |
| 5 | 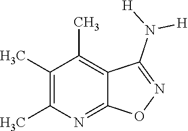 | C | B | C | D |
| 24 | 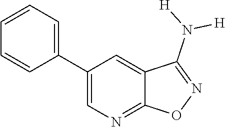 [SMILES: Nc1noc2ncc(-c3ccccc3)cc12] | D | C | B | D |
| 1 | 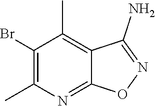 | B | A | C | D |
| 9 | 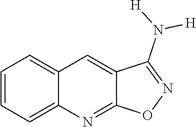 [SMILES: Nc1noc2nc3ccccc3cc12] | C | B | B | C |
| 10 | 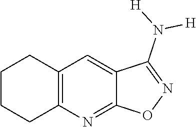 [SMILES: Nc1noc2nc3c(cc12)CCCC3] | D | B | C | D |
| 12 | 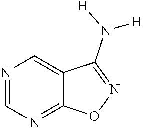 | D | B | B | D |
| 14 | 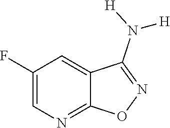 [SMILES: Nc1noc2ncc(F)cc12] | D | B | B | D |
| 15 | 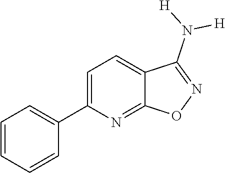 [SMILES: Nc1noc2nc(-c3ccccc3)ccc12] | D | B | C | D |
| 74 | 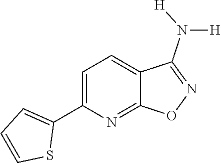 [SMILES: Nc1noc2nc(-c3cccs3)ccc12] | D | B | C | D |
| 41 | 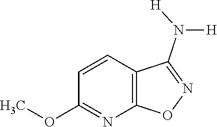 | D | B | C | D |
| 47 | 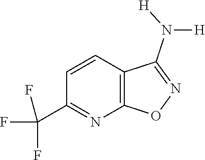 [SMILES: Nc1noc2nc(C(F)(F)F)ccc12] | A | B | B | D |
| 42 | 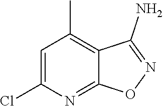 | A | A | B | D |
| 45 | 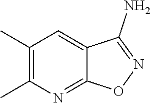 | D | C | B | D |
| 44 | 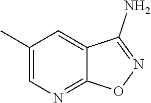 | D | C | A | D |
| 84 | 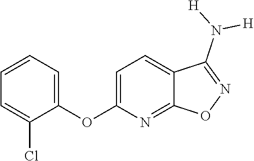 [SMILES: Nc1noc2nc(Oc3ccccc3Cl)ccc12] | C | C | B | D |
| 89 | 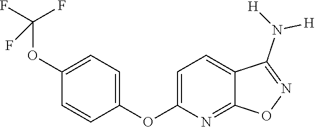 [SMILES: Nc1noc2nc(Oc3ccc(OC(F)(F)F)cc3)ccc12] | C | C | C | D |
| 95 | 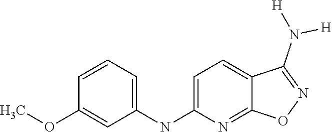 [SMILES: COc1cccc(Nc2ccc3c(N)noc3n2)c1] | D | C | C | D |
| 15B | 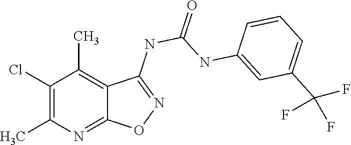 [SMILES: Cc1nc2onc(NC(=O)Nc3cccc(C(F)(F)F)c3)c2c(C)c1Cl] | D | B | C | D |
| 4B | 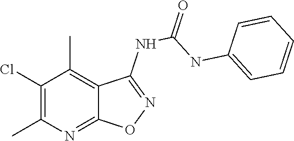 [SMILES: Cc1nc2onc(NC(=O)Nc3ccccc3)c2c(C)c1Cl] | B | A | C | D |

Activity IC₅₀ ranges: A: <1 μM, B: 1-10 μM, C: 10-100 μM, D: >100 μM

### Example 2—In Vitro Efficacy on Parental and Cisplatin-Resistant Human-Derived Lung Cancer Cells

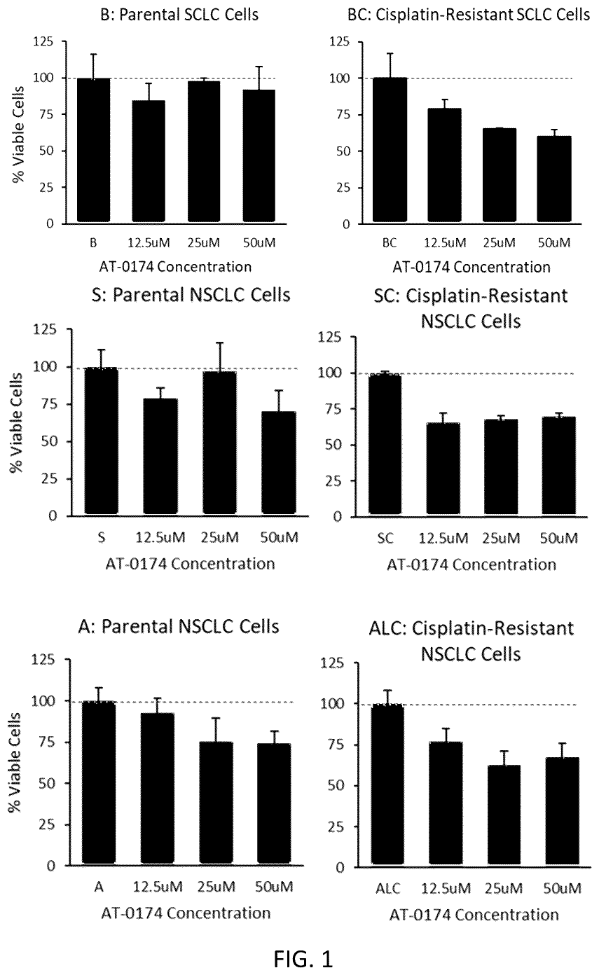

In this study 3 different human-derived lung cancer cell lines were exposed to vehicle control (parental cells B, S and A) or long-term incubation with cisplatin (BC, SC and ALC cells). Long-term incubation with cisplatin induced cisplatin-resistance in the BC, SC and ALC cell lines. AT-0174 killed cisplatin-treated cells with greater efficacy and potency than parental cells, indicating that AT-0174 would be more efficacious in patients who have developed cisplatin-resistance (see Table 2 and FIG. 1).

### Methods

### Growth Inhibition and Cytotoxicity Assay

Cells were seeded in 24-well dishes and treated with various concentrations of inhibitors (i.e. Epacadostat or AT-0174). Briefly, the culture media as well as the trypsinized cells were collected and this mixture was centrifuged at 400×g for 5 min. The supernatant was discarded, re-suspended in 1 mL of Hank's buffer, and assayed for live cells and death cells using trypan blue exclusion method.

### Statistical Analysis

Data from in vitro experiments were performed from three separate biological replicates which were isolated and analyzed in technical triplicates. Separate measurements using the two-tailed t-test and the results were expressed as mean±standard deviation.

### Results

TABLE 2
Increasing concentrations of AT-0174 induce cytotoxicity to
cisplatin-sensitive and cisplatin-resistant cells, in vitro.

| Cell | Cisplatin | | 12.5 μM | 25.0 μM | 50.0 μM |
|---|---|---|---|---|---|
| Designation | Resistant | Control | AT-0174 | AT-0174 | AT-0174 |
| B | No | 100 ± 16.2 | 84.8 ± 11.3 | 97.9 ± 2.1 | 92.2 ± 15.6 |
| S | No | 100 ± 11.3 | 78.0 ± 7.1 | 96.8 ± 19.1 | 70.2 ± 14.0 |
| A | No | 100 ± 7.8 | 92.8 ± 8.5 | 75.1 ± 14.1 | 74.4 ± 7.1 |
| BC | Yes | 100 ± 17.0 | 78.8 ± 6.4 | 65.3 ± 0.7 | 59.9 ± 4.9 |
| SC | Yes | 100 ± 1.6 | 65.7 ± 6.4 | 68.1 ± 2.1 | 70.0 ± 2.1 |
| ALC | Yes | 100 ± 9.2 | 76.7 ± 4.2 | 62.5 ± 4.9 | 67.3 ± 13.4 |

### Example 3—Inhibiting the Kynurenine Pathway In Vivo with an IDO1/TDO Dual Inhibitor (AT-0174) to Mitigate Immune Suppression in the Tumor Microenvironment of Cisplatin Resistant Lung Cancer

This study investigated and compared the in vivo antitumor effect of the selective IDO1 inhibitor epacadostat and the dual IDO1/TDO inhibitor AT-1074 on cisplatin resistant (CR) cell tumours in a syngeneic mouse model of lung cancer.

### Methods

### Cell Lines and Reagents

Cisplatin resistant mouse cell lines (LLC-CR) were derived from LLC (mouse Lewis lung cancer cells (epidermoid carcinoma), ATCC) through treatment with increasing doses of cisplatin intermittently. LLC-CR clones were maintained in half GI₅₀ dosage concentration. Briefly, parental LLC cells were seeded (4×10⁴) in 6 well plates and treated with clinical grade cisplatin (Miami VA Hospital) for 24 h with the 50% growth inhibition (GI₅₀) concentration of 0.4 μg/ml. The cultures were observed daily and allowed to grow until they reached an initial cell density. One to 2-fold increases in GI₅₀ concentration were observed within 1-2 weeks. After the cells recovered from cisplatin toxicity, they were treated again with increasing dosage of cisplatin for 24 h. These cells are seeded at 500-1000 cells per dish and cultured for 5-7 days. Clones are selected and expanded as well as tested for cisplatin sensitivity. Similar processes of exposure are carried out for a third and fourth time to generate higher resistant clones. At the end, the resistance clones (LLC-CR) were maintained in media completed with 1 μg/ml (half GI₅₀ dosage) of cisplatin. LLC-CR were developed to possess 3-5-fold resistance to cisplatin and carboplatin (˜2 μg/ml and 1.3 μg/ml, respectively), with concomitantly increased IDO1 activity and sensitivity to the IDO1 inhibitor (Nguyen D J M et al., Mol Cancer Res. 2020:18(1):105-117).

### Animal Studies

LLC or LLC-CR (1×10⁶) cells were subcutaneously injected into the flank of C57B6/J male and female mice (5 mice per group, age 6-8 weeks, Jackson laboratory) and allowed to grow for 3 days (average tumor size=50 mm³) before being treated with the selective IDO1 inhibitor epacadostat at 200 mg/kg daily or with AT-0174 at 170 mg/kg daily (a dual inhibitor of IDO1 and TDO enzymes). Both compounds were administered via oral gavage (P.O.) and diluted with 95% water+5% methylcellulose (0.5%) and stored at −20° C. Compound was prepared every 5 days. Methyl cellulose (95+5%) was used as control (“Veh”). Tumor growth was evaluated every 2 days by measuring tumor volume with calipers according to the following formula: tumor volume=width²×length×0.5. At the end of the study, mice were sacrificed and the blood, tumor, and lymph nodes were collected and processed for KYN and tryptophan (TRP) measurement and immune cell assessment respectively.

### Flow Cytometry Immune Cell Analysis

Tumors or lymph nodes were collected from mice and homogenized using syringe plungers. Mesh filters (40 μM) were placed on 15 mL or 50 mL tubes and the tissue suspensions were filtered. Cell filtrates were spun down at 1000 g. Red blood cell lysis buffer (1% ammonium oxalate) was added into the cell pellets and resuspended. Cell pellets were washed using PBS and isolated. Tumors were isolated using EasySep™ Mouse TIL (CD45) Positive Selection Kit (Stemcell Technologies). To detect regulatory T cells (T-regs) or natural killer cells (NKs), EasySep™ Mouse CD25 Regulatory T Cell Positive Selection Kit and EasySep™ Mouse CD49b Positive Selection Kit were used, respectively. Isolated cells were stained according to manufacturer's instructions. Cells were also fixed with 2% paraformaldehyde and analysis was done using the CytoFLEX Flow Cytometer (Beckman). All flow antibodies were obtained from Biolegend.

### KYN and TRP Measurements

Blood samples were collected via cardiac puncture from mice at the time of sacrifice and the Biochrom 30 amino acid analyzer (using ion exchange chromatography) was used to analyze free amino acids in samples. Briefly, 200 μL of serum extract and equal volume of 30% v/v deproteinization agent (sulfosalicylic acid) were mixed and vortexed for 10 s. Samples were then incubated at 55° C. for 20 min, spin down at 2500 g for 20 min at 4° C. Supernatant were collected, filtered through a 0.22 μm filter, and injected into the Biochrom 30. Values were reported compared to normal ranges in controls. Samples used in ion exchange chromatography are separated in an analytical fashion with an ion exchange column and buffers of increasing pH and ionic strength. After post-column derivatization with Ninhydrin, and colorimetric intensity recordings at 570 nm and 440 nm, the amino acids separated by chromatography were detected. Amino acid concentration was calculated by comparing the peak area of a particular amino acid to the peak area of an internal standard-AEC of known concentration, and then multiplying by its specific response factor from calibration.

### Results

### Effect of IDO1 or Dual IDO1/TDO Inhibition on the Tumor Growth in Syngeneic Mouse Models.

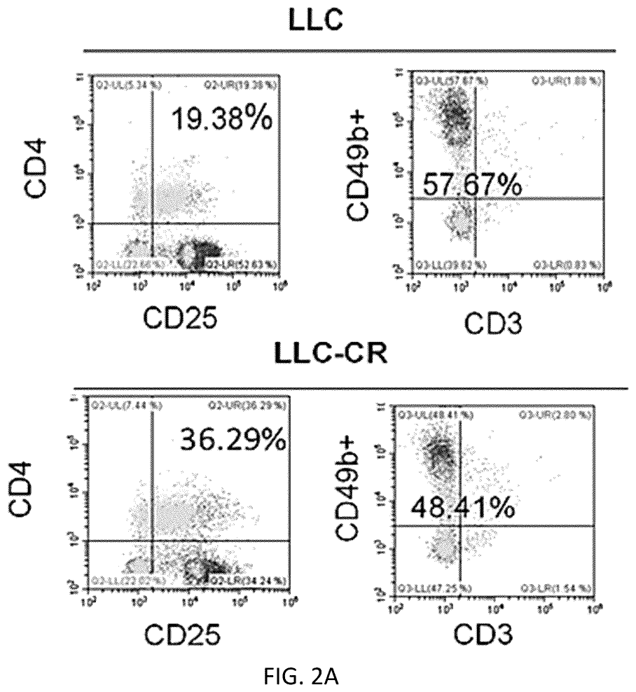

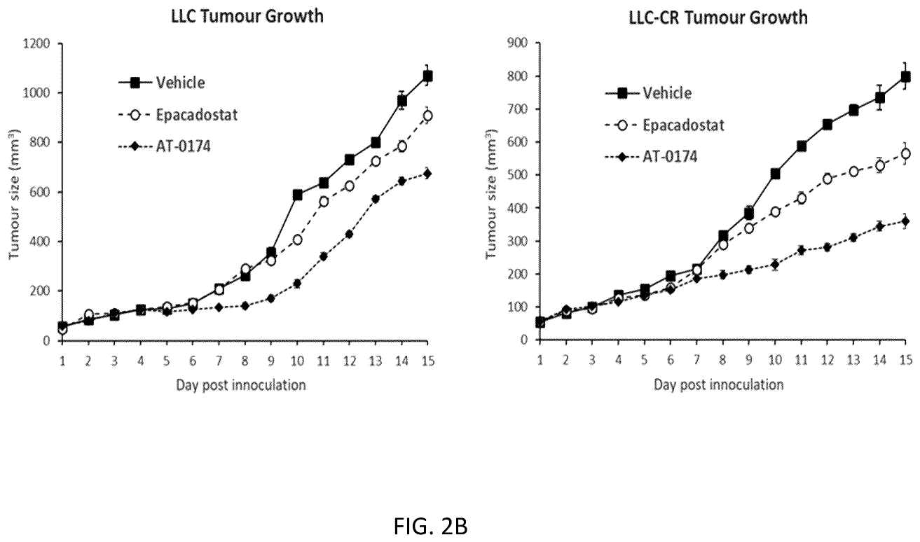

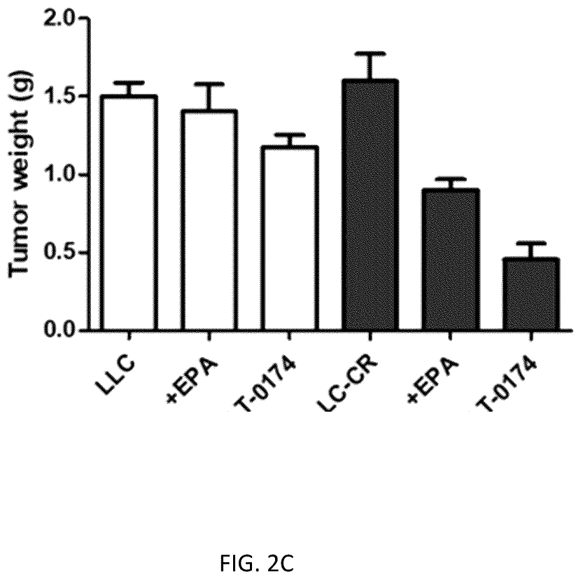

In this example, LLC-CR from the Lewis lung mouse cell line (LLC; see methods) was used to determine, in vivo, the effect of IDO1 or dual IDO1/TDO inhibition on tumor growth. Mice were inoculated with either LLC or LLC-CR and treated with epacadostat (200 mg/kg P.O. 1/day) or AT-0174 (170 mg/kg P.O. 1/day). C57B6/J mice allografted with LLC-CR showed an increase in T-reg (CD4′ CD25′) population demonstrating the role of the kynurenine pathway (KP) and the immunosuppressive tumor microenvironment (TME) in CR tumors (n=5; p<0.02) (FIG. 2A).

Selective IDO1 inhibition with epacadostat diminished tumor growth in CR allografts (Table 3 and FIG. 2B) with a reduction of the total tumor weight when compared with LLC mouse tumors. However, AT-0174 induced a significantly greater marked reduction in tumor size (Table 3 and FIG. 2B), as well as tumor weight in LLC-CR allograft mice (FIG. 2C). Together, the data indicated that the dual IDO1/TDO inhibitor was superior to the selective IDO1 inhibitor in inhibiting the kynurenine pathway.

TABLE 3
AT-0174 and epacadostat on tumor growth of LLC and LLC-CR tumors
in mice (tumor volume shown, mm³), showing greatest effect is seen
with AT-0174 in cisplatin-resistant lung cancer cell tumors.

| | | |
|---|---|---|
| Control | AT-0174 | Epacadostat |

| | | | | | | |
|---|---|---|---|---|---|---|
| Day | Mean | SEM | Mean | SEM | Mean | SEM |

LLC (cisplatin-sensitive cell tumours)

| | | | | | | |
|---|---|---|---|---|---|---|
| 1 | 57.0 | 2.0 | 58.0 | 2.5 | 47.0 | 3.0 |
| 2 | 86.0 | 2.9 | 79.0 | 3.3 | 108.0 | 5.8 |
| 3 | 105.0 | 3.2 | 110.0 | 3.2 | 110.0 | 4.5 |
| 4 | 124.0 | 4.0 | 125.0 | 2.2 | 124.0 | 1.9 |
| 5 | 129.0 | 2.9 | 115.2 | 4.4 | 137.0 | 3.0 |
| 6 | 149.0 | 2.9 | 124.0 | 1.9 | 151.0 | 1.9 |
| 7 | 210.0 | 4.5 | 135.0 | 2.2 | 208.0 | 3.7 |
| 8 | 265.0 | 7.4 | 140.6 | 1.7 | 290.0 | 11.8 |
| 9 | 355.0 | 20.0 | 171.0 | 5.6 | 325.0 | 11.2 |
| 10 | 590.0 | 12.7 | 230.0 | 9.4 | 409.0 | 16.8 |
| 11 | 638.0 | 10.8 | 340.0 | 18.7 | 563.0 | 13.8 |
| 12 | 732.0 | 9.2 | 430.0 | 14.6 | 625.0 | 11.2 |
| 13 | 800.0 | 15.8 | 571.8 | 9.5 | 725.0 | 11.2 |
| 14 | 970.0 | 37.4 | 645.0 | 22.9 | 785.0 | 15.0 |
| 15 | 1070.0 | 40.6 | 675.0 | 33.5 | 910.0 | 23.2 |

LLC-CR (cisplatin-resistant cell tumours)

| | | | | | | |
|---|---|---|---|---|---|---|
| 1 | 54.0 | 2.9 | 57.0 | 2.0 | 57.0 | 3.0 |
| 2 | 81.0 | 1.9 | 93.0 | 3.7 | 89.0 | 5.1 |
| 3 | 100.0 | 3.2 | 102.0 | 3.7 | 96.0 | 2.4 |
| 4 | 135.0 | 2.2 | 115.0 | 4.5 | 126.0 | 2.9 |
| 5 | 154.0 | 1.9 | 135.0 | 2.2 | 137.0 | 3.4 |
| 6 | 194.0 | 7.9 | 153.0 | 3.0 | 158.0 | 4.6 |
| 7 | 216.0 | 5.1 | 185.0 | 4.5 | 212.0 | 3.7 |
| 8 | 318.0 | 9.3 | 198.0 | 2.0 | 291.0 | 4.6 |
| 9 | 385.0 | 15.0 | 213.0 | 4.4 | 340.0 | 6.3 |
| 10 | 505.0 | 20.0 | 228.0 | 3.4 | 390.0 | 6.1 |
| 11 | 588.0 | 15.9 | 272.0 | 20.4 | 430.0 | 9.4 |
| 12 | 655.0 | 25.5 | 280.0 | 14.6 | 490.0 | 12.7 |
| 13 | 698.0 | 19.9 | 310.0 | 10.0 | 511.0 | 17.2 |
| 14 | 735.0 | 10.0 | 345.0 | 16.6 | $30.0 | 14.6 |
| 15 | 800.0 | 17.7 | 360.0 | 20.3 | 565.0 | 18.7 |

Tumor Immune Cell and TRP/KYN Assessments

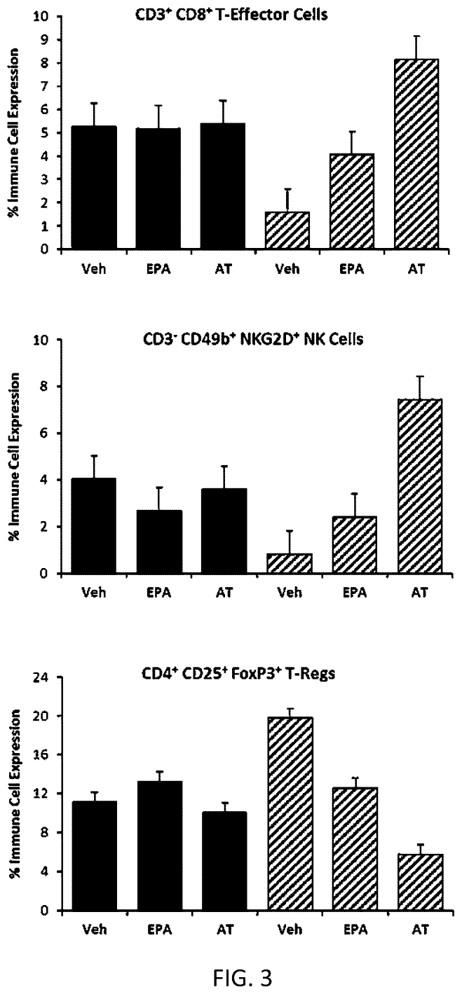

Anti-tumor immunity is reliant on the checks and balances between cytotoxic cells (e.g. NK) and immune suppressive cells (e.g. T-regs). As shown in FIG. 2A, LLC-CR possessed significantly higher T-regs frequency, thus suggesting that LLC-CR adapted to evade immune surveillance. To determine whether the inhibition of the kynurenine pathway could restore anti-tumor immunity in the CR tumor model, T-effector, T-reg and NK populations after epacadostat (EPA) or AT-0174 (AT) treatments were assayed. Populations of T-regs (CD4⁺ CD25⁺ FoxP3⁺) were significantly reduced in LLC-CR; but notably, while T-effector cells (CD3⁺ CD8⁺) and activated NK cells (CD3⁻ CD49b⁺ NKG2D⁺) were found to be significantly higher in treatment groups (Table 4 and FIG. 3). In all cases, AT-1074 had a greater pro-immune effect on these immune cell populations than the selective IDO1 inhibitor epacadostat.

TABLE 4
Effect of AT-0174 and epacadostat on immune cell expression in LLC and LLC-CR tumors in mice,
showing greatest effect is seen with AT-0174 in cisplatin-resistant lung cancer cell tumors

| | | LLC | | | LLC-CR | | |
|---|---|---|---|---|---|---|---|
| | | Vehicle | Epacadostat | AT-0174 | Vehicle | Epacadostat | AT-0174 |
| CD3⁺ CD8⁺ T-Effector | | 5.26 ± 0.56 | 5.16 ± 0.65 | 5.38 ± 0.79 | 1.60 ± 0.39 | 4.06 ± 0.63 | 8.13 ± 0.94 |
| CD4⁺ CD25⁺ FoxP3⁺ | T-regs | 11.12 ± 1.00 | 13.27 ± 0.83 | 10.05 ± 0.79 | 19.79 ± 1.03 | 12.60 ± 0.94 | 5.75 ± 1.53 |
| CD3⁺ CD49⁺ NKG2D⁺ | NK Cells | 4.03 ± 0.36 | 2.66 ± 0.57 | 3.58 ± 0.70 | 0.82 ± 0.19 | 2.38 ± 0.42 | 7.44 ± 0.62 |

This is important since it has been shown that kynurenine not only plays a critical role in the reprogramming of naïve T cells to T-reg cells; kynurenine can also impair NK cells' ability to kill target cells (Della Chiesa M, Carlomagno S, Frumento G, Balsamo M, Cantoni C, Conte R, et al. The tryptophan catabolite L-kynurenine inhibits the surface expression of NKp46- and NKG2D-activating receptors and regulates NK-cell function. Blood. 2006; 108(13):4118-25.). Thus, the data clearly indicated that AT-0174 can enhance activity of NK cells by modulating the NKG2D receptor in cisplatin-resistant cells.

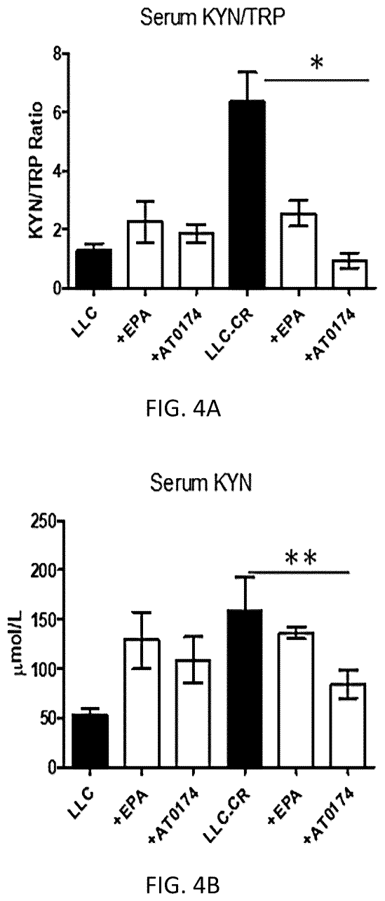

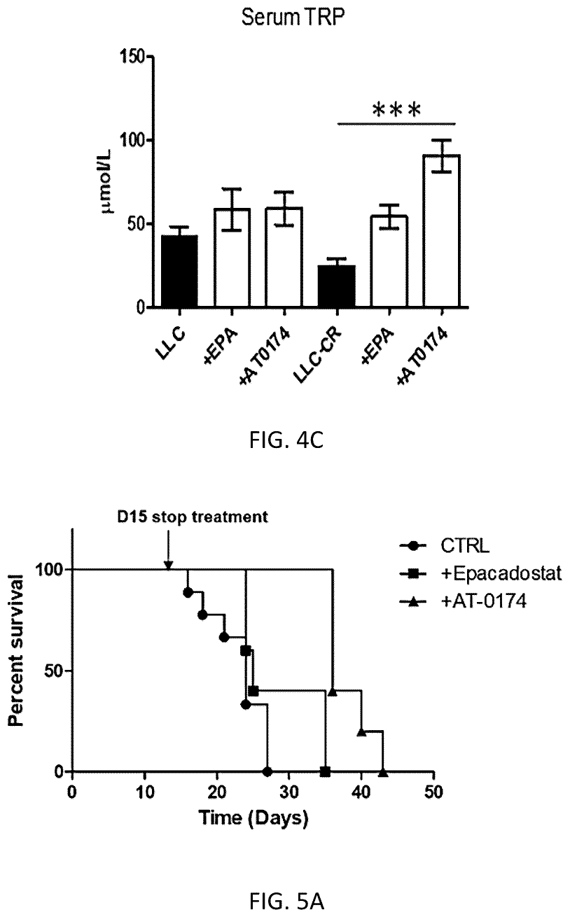

Consistent with this finding, serum KYN/TRP ratio, a biomarker of the IDO1 activity, was significantly decreased in LLC-CR treated with AT-0174 (FIGS. 4A-4C). Concomitantly, increase serum TRP levels were found in AT-0174 when compared to the epacadostat treatment group, confirming a sustained in vivo inhibition of Trp degradation by TDO in the normal tissues.

TABLE 5
Effect of AT-0174 and epacadostat on tryptophan, kynurenine
and the KYN/TRP ratio in LLC and LLC-CR tumors in mice

| | LLC | | | LLC-CR | | |
|---|---|---|---|---|---|---|
| | Vehicle | Epacadostat | AT-0174 | Vehicle | Epacadostat | AT-0174 |
| Kynurenine | 52.8 ± 2.8 | 74.6 ± 7.7 | 64.2 ± 9.3 | 138.8 ± 18.2 | 81.8 ± 11.9 | 50 ± 9.7 |
| Tyrptophan | 42.0 ± 2.8 | 58.6 ± 5.6 | 59.2 ± 4.4 | 24.8 ± 1.8 | 43 ± 5.8 | 85.8 ± 8.3 |
| KYN/TRP Ratio | 1.28 ± 0.11 | 1.35 ± 0.23 | 1.11 ± 0.17 | 4.85 ± 0.77 | 2.01 ± 0.32 | 1.18 ± 0.31 |

### Example 4—Effect of AT-0174 on Survival of Mice with Cisplatin-Resistant Syngeneic Lung Cancer Tumors, with and without Co-Treatment with Anti PD-1 Antibodies

To investigate the synergy of immune response on tumors between the dual IDO1/TDO inhibitor AT-0174 and an immune checkpoint inhibitor, the compound was tested in mice with cisplatin-resistant lung cancer tumors with and without with anti-PD1 antibody co-treatment.

LLC-CR (1×10⁶) cells were subcutaneously injected into the flank of C57B6/J mice (6 mice per group, age 8 weeks) and allowed to grow for 3 days (average tumor size=50 mm³) before being treated with IDO1 inhibitor epacadostat at 200 mg/kg daily (a supra-maximal dose) or with AT-0174 at 170 mg/kg. Both compounds were administered via oral gavage (P.O.) in 95% water+5% methylcellulose (0.5%). Anti-mouse PD-1 MAb clone RMPi-14 was used as checkpoint inhibitor and administered via intraperitoneal injection (i.p.) at a dose of 10 mg/kg every 3 days.

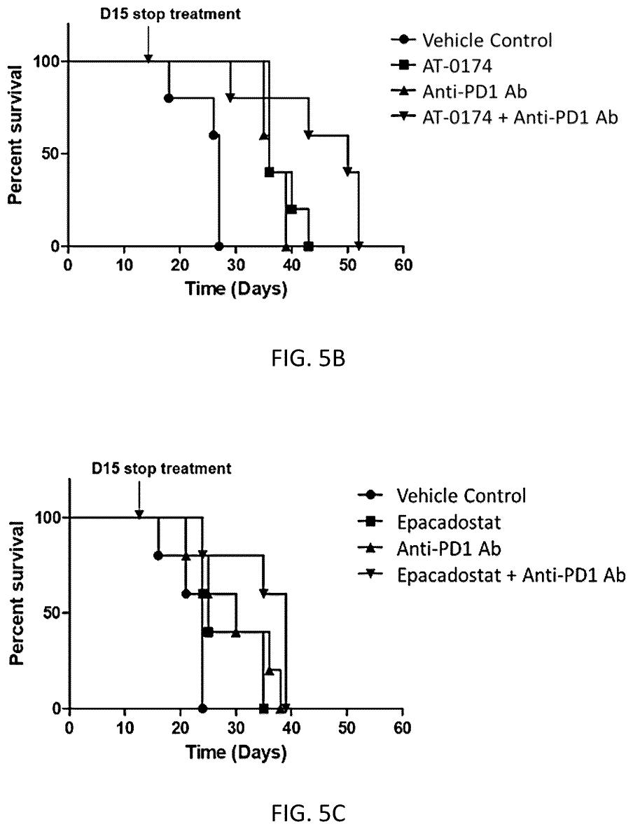

Comparing the effect of the dual inhibitor AT-0174 with the selective IDO1 inhibitor epacadostat when each were given alone, it was observed that AT-0174 increased survival to a greater extent than the selective agent (Table 6 and FIG. 5A). The anti-PD1 antibody given alone had a similar effect on survival as AT-0174 or epacadostat, but it only appeared to provide marked synergy with AT-0174 (FIG. 5B) with a lesser synergistic effect evident with epacadostat (FIG. 5C). These data demonstrate that the effects previously observed on tumour growth and immune cell changes with the tumor microenvironment translate to a survival benefit in the same chemotherapy-resistant mouse model.

TABLE 6
Survival data of mice with cisplatin-resistant
LLC tumors following various drug treatments

| | | | Median | Maximal |
|---|---|---|---|---|
| Treatment | N | First Death | Survival Time | Survival |
| Vehicle control | 12 | 16 days | 24 days | 27 days |
| Epacadostat | 6 | 24 days | 25 days | 35 days |
| Epacadostat + Anti PD1 | 6 | 24 days | 39 days | 39 days |
| AT-0174 | 6 | 36 days | 36 days | 43 days |
| AT-0174 + Anti PD1 | 6 | 29 days | 50 days | 52 days |

### Example 5—Evaluation of the In Vivo Efficacy of AT-0174 in a Mouse Subcutaneous Pan02 Syngeneic Model of Pancreatic Cancer

In this study, the efficacy of the test compound AT-0174 combined with Oxaliplatin was evaluated in the treatment of Pan02 syngeneic model in female C57BL/6 mice.

### Methods and Materials

### Abbreviations

Dosing schedule

| | |
|---|---|
| Qd | Every day |
| BIW | Twice weekly |

Route of administration (ROA)

| | |
|---|---|
| i.p. | Intraperitoneal (ly) |
| p.o. | Oral(ly) |
| BW | Body weight |
| BWL | Body weight loss |

### Study Materials

Oxaliplatin (Action: Positive Control; Storage conditions: Room temperature)
AT-0174 (Action: Test Article; Storage conditions: 2-8° C.)
5% Glucose (Action: Vehicle for oxaliplatin)
5% DMSO+95% water (Action: Vehicle for AT-0174)

### Animals

Female C57BL/6 mice were used in the study, with ad libitum access to rodent food and water throughout the study period.

### Cell Culture and Inoculation

Murine pancreatic Pan02 cancer cells were cultured in a 37° C. incubator containing 5% CO₂ with 10% FBS in DMEM medium. The cells were sub-cultured within 10 passages before inoculated into the mice. 1*10⁶ PANC02 cells in 100 μL serum-free medium mixed with Matrigel (v/v 1:1) were subcutaneously inoculated at the right flank of each mouse under anesthetized with 2-5% isoflurane before inoculation.

Treatment Groups The dosing regimen is indicated in Table 7.

TABLE 7
Grouping and dosing regimen

| Group | N | Test Articles | Dose (mg/kg) | Dosing Regimen |
|---|---|---|---|---|
| 1 | 6 | Vehicle 1 | N/A | i.p. BIW*10 times |
| 2 | 6 | Oxaliplatin | 6 | i.p. BIW*10 times |
| 3 | 6 | Oxaliplatin + | 6 + 120 | i.p. BIW*10 times + |
| | | AT-0174 | | p.o. QD*Day 7-32 |

N: animal number per group

Dosing volume: adjusted dosing volume based on body weight (10 uL/g)

### Test Article Dosing Solution Preparation

### Detailed Instructions on Formulation and Storage

| | | Concentration |
|---|---|---|
| Compound | Preparation instruction | (mg/mL) |
| Oxaliplatin | Weighed 2.40 mg Oxaliplatin, added in 4.00 mL 5% | 0.6 |
| 6 mg/kg | Glucose, mixed well to get a clear solution. | |
| AT-0174 | Weighed 72.00 mg AT-0174, added in 0.30 mL | 12 |
| 120 mg/kg | DMSO, mixed well and then added 5.70 mL water, | |
| | and mixed well | |

### Clinical Observations

Each mouse was observed for changes in general appearance and behavior every day after grouping for 4 weeks. After finishing administration, mice were observed for another week.

### Measurements Taken

Body weight: Mice body weights were measured and recorded three times per week after grouping. Animals were also weighed in the event of accidental death or near-death euthanasia.

Tumor volume: The tumor volume (V) was calculated as follows: V=(length×width²)/2. Tumor volume was measured and recorded three times per week after grouping.

The individual relative tumor volume (RTV) was calculated as follows: RTV=Vt/V0, where Vt is the volume on each day, and V0 is the volume at the beginning of the treatment.

### FACS Analysis

On Day 34, all tumor-bearing mice were sacrificed, and tumors were collected for FACS analysis. FACS panels were:

Panel A: L/D, CD45, CD3, CD4, CD8, CD11b, CD25, IFN-γ, Foxp3.

Panel B: L/D, CD45, CD3, CD11b, NKG2D, F4/80.

Flow cytometry was conducted using a Beckman Cytoflex LX and antibodies from Thermofisher and Biolegend, using standard methods.

### Statistics

Comparisons between two groups were made by Dunnett's multi-comparison test, p<0.05 was considered as significant.

### Results

### Body Weight and Clinical Observations

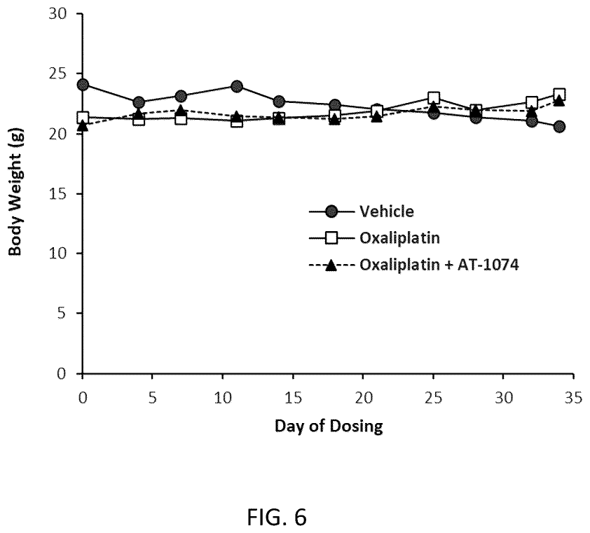

All animals tolerated dose treatments well. Slight body weight loss was observed when Oxaliplatin mono-treatment and recovered after Day 14. Body weights of the mice are shown in Table 8 and FIG. 6. These results confirm that mice tolerated administration of the Oxaliplatin and Oxaliplatin+AT-0174 without significant weight loss compared to the control.

TABLE 8
Body weight (g) of mice with syngeneic Pan02
tumors, following administration of either vehicle,
oxaliplatin or oxaliplatin plus AT-0174

| | | |
|---|---|---|
| | | Oxaliplatin + |
| Vehicle | Oxaliplatin | AT-0174 |

| Day | Mean | SEM | Mean | SEM | Mean | SEM |
|---|---|---|---|---|---|---|
| 0 | 20.67 | 0.37 | 21.40 | 0.43 | 20.68 | 0.38 |
| 4 | 21.05 | 0.46 | 21.21 | 0.37 | 21.69 | 0.40 |
| 7 | 21.37 | 0.38 | 21.33 | 0.51 | 21.99 | 0.40 |
| 11 | 21.77 | 0.37 | 21.10 | 0.47 | 21.42 | 0.59 |
| 14 | 22.03 | 0.42 | 21.28 | 0.43 | 21.40 | 0.50 |
| 18 | 22.38 | 0.53 | 21.50 | 0.50 | 21.26 | 0.53 |
| 21 | 22.75 | 0.66 | 21.93 | 0.47 | 21.46 | 0.58 |
| 25 | 23.96 | 0.61 | 23.00 | 0.44 | 22.29 | 0.62 |
| 28 | 23.18 | 0.73 | 22.00 | 0.69 | 22.00 | 0.56 |
| 32 | 22.66 | 1.30 | 22.65 | 0.49 | 21.94 | 0.80 |
| 34 | 24.13 | 0.73 | 23.31 | 0.51 | 22.78 | 0.61 |

### Tumor Volume (TV) and Relative Tumor Volume (RTV)

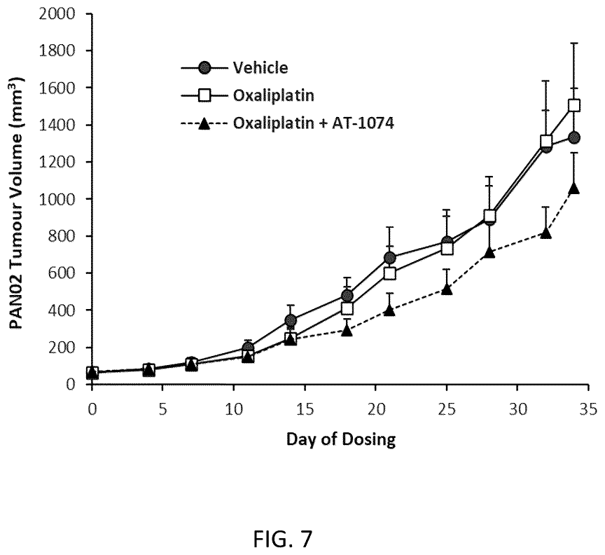

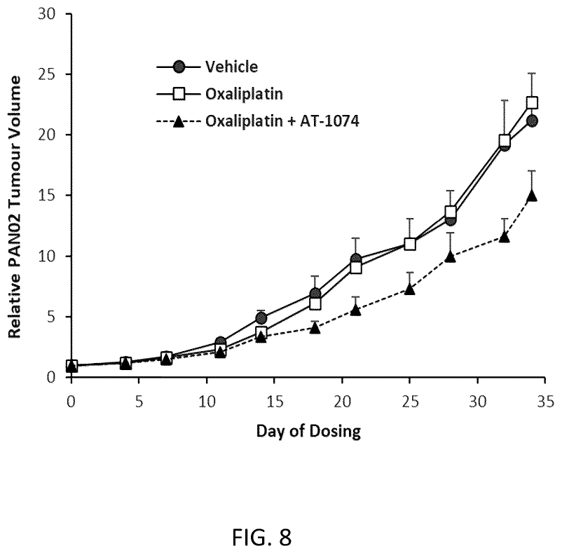

The results of tumor volume in the tumor bearing mice were shown in Table 9 and FIG. 7. The results of relative tumor volume in the tumor bearing mice are shown in Table 10 and FIG. 8. These data show that, compared with the vehicle group, the chemotherapy oxaliplatin at 6 mg/kg BIW provided little anti-tumor response during the dosing period, with the small effect seen early in the treatment period lost beyond day 12. Compared with the vehicle group, the test compound AT-0174 at 120 mg/kg QD combined with oxaliplatin at 6 mg/kg BIW showed a marked decrease in tumor volume by day 32.

TABLE 9
Tumor Volume (mm³) of mice with syngeneic Pan02
tumors, following administration of either vehicle,
oxaliplatin or oxaliplatin plus AT-0174

| | | | | | Oxaliplatin + | |
|---|---|---|---|---|---|---|
| | Vehicle | | Oxaliplatin | | AT-0174 | |
| Day | Mean | SEM | Mean | SEM | Mean | SEM |
| 0 | 67.40 | 5.70 | 63.84 | 6.23 | 69.46 | 5.76 |
| 4 | 85.19 | 12.42 | 80.36 | 15.64 | 84.57 | 14.62 |
| 7 | 119.66 | 24.71 | 108.77 | 20.43 | 109.01 | 19.67 |
| 11 | 198.28 | 38.51 | 152.24 | 26.68 | 151.11 | 31.99 |
| 14 | 345.70 | 83.38 | 250.08 | 59.31 | 242.64 | 55.82 |
| 18 | 481.19 | 96.71 | 410.18 | 115.88 | 295.18 | 56.50 |
| 21 | 683.25 | 164.75 | 601.42 | 144.14 | 402.82 | 89.07 |
| 25 | 768.79 | 172.76 | 735.54 | 171.43 | 517.86 | 103.92 |
| 28 | 891.04 | 178.42 | 912.95 | 207.78 | 714.27 | 157.13 |
| 32 | 1283.65 | 194.34 | 1316.29 | 317.72 | 818.21 | 137.12 |
| 34 | 1334.04 | 261.93 | 1507.92 | 332.91 | 1059.82 | 191.37 |

TABLE 10
Relative tumor volume of mice with syngeneic Pan02
tumors, following administration of either vehicle,
oxaliplatin or oxaliplatin plus AT-0174

| | | |
|---|---|---|
| | | Oxaliplatin + |
| Vehicle | Oxaliplatin | AT-0174 |

| Day | Mean | SEM | Mean | SEM | Mean | SEM |
|---|---|---|---|---|---|---|
| 0 | 1.00 | 0.00 | 1.00 | 0.00 | 1.00 | 0.00 |
| 4 | 1.24 | 0.08 | 1.22 | 0.17 | 1.19 | 0.12 |
| 7 | 1.72 | 0.19 | 1.64 | 0.21 | 1.51 | 0.15 |
| 11 | 2.89 | 0.43 | 2.33 | 0.30 | 2.08 | 0.29 |
| 14 | 4.89 | 0.75 | 3.72 | 0.64 | 3.32 | 0.50 |
| 18 | 6.96 | 1.11 | 6.11 | 1.39 | 4.12 | 0.52 |
| 21 | 9.72 | 1.73 | 9.09 | 1.75 | 5.61 | 0.99 |
| 25 | 11.04 | 1.94 | 11.01 | 2.02 | 7.32 | 1.28 |
| 28 | 12.99 | 2.31 | 13.70 | 2.43 | 9.96 | 1.92 |
| 32 | 19.22 | 3.01 | 19.54 | 3.59 | 11.59 | 1.53 |
| 34 | 21.22 | 3.92 | 22.69 | 3.82 | 15.01 | 2.01 |

### FACS Analysis Results

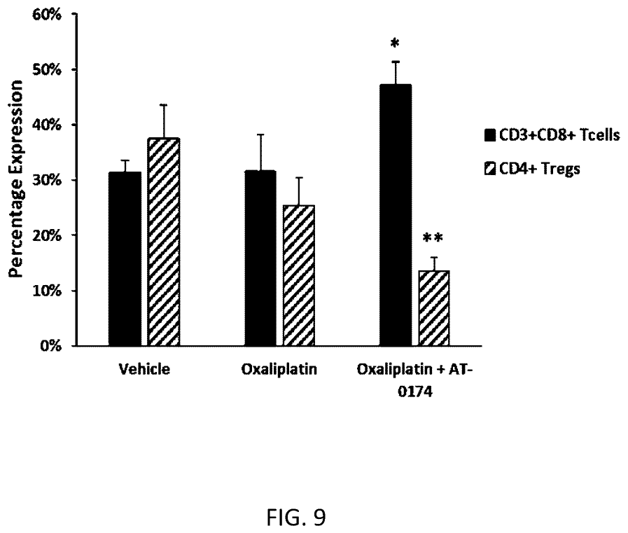

Immune phenotyping in PanO2 tumors is shown in Table 11 and FIG. 9.

This data shows that combination treatment with oxaliplatin+AT-0174 had a greater effect on immune cell changes than oxaliplatin alone, significantly attenuating Treg expression.

TABLE 11
Immune cell changes in response to oxaliplatin or oxaliplatin plus
AT-0174, in the tumor microenvironment of Pan02 tumors in mice

| | | | Oxaliplatin + |
|---|---|---|---|
| | Vehicle control | Oxaliplatin | AT-0174 |
| CD4+ Tregs | 37.44% | 25.40% | 13.50% |
| CD8+ CD3+ Tcells | 31.44% | 31.62% | 47.22% |

When a group of materials, compositions, components or compounds is disclosed herein, it is understood that all individual members of those groups and all subgroups thereof are disclosed separately. When a Markush group or other grouping is used herein, all individual members of the group and all combinations and subcombinations possible of the group are intended to be individually included in the disclosure. Every formulation or combination of components described or exemplified herein can be used to practice the invention, unless otherwise stated.

All references cited herein are hereby incorporated by reference in their entirety to the extent that there is no inconsistency with the disclosure of this specification. Some references provided herein are incorporated by reference to provide details concerning sources of starting materials, additional starting materials, additional reagents, additional methods of synthesis, additional methods of analysis, additional biological materials, additional cells, and additional uses of the invention. All headings used herein are for convenience only. All patents and publications mentioned in the specification are indicative of the levels of skill of those skilled in the art to which the invention pertains and are herein incorporated by reference to the same extent as if each individual publication, patent or patent application was specifically and individually indicated to be incorporated by reference. References cited herein are incorporated by reference herein in their entirety to indicate the state of the art as of their publication or filing date and it is intended that this information can be employed herein, if needed, to exclude specific embodiments that are in the prior art.

Although the invention has been described by way of example and with reference to particular embodiments, it is to be understood that modifications and/or improvements may be made without departing from the scope of the invention.

1. A method of treating refractory cancer or preventing the development of refractory cancer in a subject, the method comprising administering to the subject in need thereof a therapeutically effective amount of a dual inhibitor of indoleamine-2,3-dioxygenase (IDO1) and tryptophan-2,3-dioxygenase (TDO).

2. Use of a dual inhibitor of IDO1 and TDO in the manufacture of a medicament for treating refractory cancer or preventing the development of refractory cancer in a subject.

3. A dual inhibitor of indoleamine-2,3-dioxygenase (IDO1) and tryptophan-2,3-dioxygenase (TDO) for use in the treatment of refractory cancer or preventing the development of refractory cancer in a subject.

4. A method of treating a refractory cancer cell or preventing the development of a refractory cancer cell, the method comprising administering a dual inhibitor of IDO1 and TDO to said cancer cell.

5. Use of a dual inhibitor of IDO1 and TDO in the manufacture of a medicament for treating a refractory cancer cell or preventing the development of a refractory cancer cell.

6. A dual inhibitor of IDO1 and TDO for use treating a refractory cancer cell or preventing the development of a refractory cancer cell.

7. The method, use or dual inhibitor of any one of claims 4 to 6 wherein the refractory cancer cell is in vitro or in vivo.

8. The method, use or dual inhibitor of any one of the preceding claims wherein the refractory cancer or cancer cell is refractory to an anticancer agent and/or cancer therapy.

9. The method, use or dual inhibitor of any one of the preceding claims wherein the refractory cancer or cancer cell is refractory to a cancer therapy that comprises a step of administering one or more anticancer agent.

10. The method, use or dual inhibitor of any one of the preceding claims wherein the subject or refractory cancer cell has previously been administered an anticancer agent or cancer therapy, and the refractory cancer or cancer cell is refractory to the anticancer agent or cancer therapy.

11. The method, use or dual inhibitor of any one of claims 8 to 10 wherein the anticancer agent is selected from: a chemotherapeutic agent or a radiotherapeutic agent.

12. The method, use or dual inhibitor of claim 11 wherein the anticancer agent is a platinum-based chemotherapeutic agent.

13. The method, use or dual inhibitor of claim 12 wherein the platinum-based chemotherapeutic agent is selected from: carboplatin, cisplatin, lobaplatin, oxaliplatin, picoplatin, nedaplatin, phenanthriplatin and satraplatin.

14. The method, use or dual inhibitor of claim 12 wherein the platinum-based chemotherapeutic agent is cisplatin, carboplatin or oxaliplatin.

15. The method, use or dual inhibitor of claim 12 wherein the platinum-based chemotherapeutic agent is cisplatin or oxaliplatin.

16. The method, use or dual inhibitor of any one of claims 1 to 8 or 10 wherein the refractory cancer or cancer cell is refractory to a radiotherapy.

17. The method, use or dual inhibitor of any one of claims 8 to 16 wherein the anticancer agent and/or cancer therapy induces ROS accumulation within cancer cells.

18. The method, use or dual inhibitor of claim 17 wherein the refractory cancer or refractory cancer cell is resistant to said ROS accumulation within the cancer cells.

19. The method, use or dual inhibitor of any one of the preceding claims wherein the refractory cancer or refractory cancer cell is colorectal cancer, breast cancer, melanoma, reproductive organ cancer, respiratory tract cancer, brain cancer, digestive tract cancer, urinary tract cancer, eye cancer, liver cancer, skin cancer, head and neck cancer, thyroid cancer, parathyroid cancer and/or their distant metastases.

20. The method, use or dual inhibitor of any one of the preceding claims wherein the refractory cancer or refractory cancer cell is lymphoma, sarcoma or leukemia.

21. The method, use or dual inhibitor of any one of the preceding claims wherein the refractory cancer or refractory cancer cell is selected from the group consisting of: refractory breast cancer selected from: invasive ductal carcinoma, invasive lobular carcinoma, ductal carcinoma in situ, and lobular carcinoma in situ; refractory cancer of the respiratory tract selected from: small-cell and non-small-cell lung carcinoma, bronchial adenoma and pleuropulmonary blastoma; refractory brain cancer selected from: glioblastoma, brain stem and hypophthalmic glioma, cerebellar and cerebral astrocytoma, medulloblastoma, ependymoma, neuroectodermal and pineal tumor; refractory tumor of the male reproductive organs selected from: prostate and testicular cancer; refractory tumor of the female reproductive organs selected from: endometrial, cervical, ovarian, ovarian adenocarcinoma, vaginal, and vulvar cancer and sarcoma of the uterus; refractory tumor of the digestive tract selected from: anal, colon, colorectal, esophageal, gallblader, gastric, pancreatic, rectal, small-intestine, and salivary gland cancer; refractory tumors of the urinary tract selected from: bladder, penile, kidney, renal pelvis, ureter, and urethral cancer; refractory Eye cancer selected from: intraocular melanoma and retinoblastoma; refractory liver cancer selected from: hepatocellular carcinoma (liver cell carcinomas with or without fibrolamellar variant), cholangiocarcinoma (intrahepatic bile duct carcinoma), and mixed hepatocellular cholangiocarcinoma; refractory skin cancer selected from: squamous cell carcinoma, Kaposi's sarcoma, malignant melanoma, Merkel cell skin cancer, and non-melanoma skin cancer; refractory head-and-neck cancer selected from laryngeal/hypopharyngeal/nasopharyngeal/oropharyngeal cancer, and lip and oral cavity cancer; refractory lymphoma selected from: AIDS-related lymphoma, non-Hodgkin's lymphoma, cutaneous T-cell lymphoma, Hodgkin's disease, and lymphoma of the central nervous system; refractory sarcoma selected from: sarcoma of the soft tissue, osteosarcoma, malignant fibrous histiocytoma, lymphosarcoma, and rhabdomyosarcoma; and refractory leukemia selected from: acute myeloid leukemia, acute lymphoblastic leukemia, chronic lymphocytic leukemia, chronic myelogenous leukemia, and hairy cell leukemia, and/or their distant metastases.

22. The method, use or dual inhibitor of any one of the preceding claims wherein the refractory cancer or refractory cancer cell is selected from lung cancer, pancreatic cancer, breast cancer and ovarian cancer.

23. The method, use or dual inhibitor of any one of the preceding claims wherein the refractory cancer is a solid tumor.

24. The method, use or dual inhibitor of claim 8 or 9 wherein the anticancer agent is a platinum-based chemotherapeutic agent and the refractory cancer is selected from: lung cancer, pancreatic cancer, breast cancer and ovarian cancer.

25. The method, use or dual inhibitor of claim 24 wherein the anticancer agent is selected from cisplatin, carboplatin or oxaliplatin.

26. The method, use or dual inhibitor of claim 25 wherein the anticancer agent is cisplatin or oxaliplatin.

27. The method, use or dual inhibitor of any one of the preceding claims wherein the dual inhibitor of IDO1 and TDO is a compound of Formula I or a pharmaceutically acceptable salt thereof, wherein: 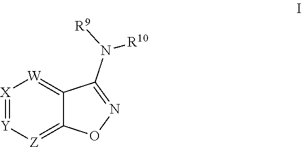
W is CR¹, N or N-oxide;
X is CR², N or N-oxide;
Y is CR³, N or N-oxide;
Z is CR⁴, N or N-oxide;
and where at least one of W, X, Y, and Z is N or N-oxide;
R¹, R², R³ and R⁴ are each independently selected from the following groups: H, halo, R, —OH, —OR, —OC(O)H, —OC(O)R, —OC(O)NH₂, —OC(O)NHR, —OC(O)NRR, —OP(O)(OH)₂, —OP(O)(OR)₂, —NO₂, —NH₂, —NHR, —NRR, —NHC(O)H, —NHC(O)R, —NRC(O)R, —NHC(O)NH₂, —NHC(O)NRR, —NRC(O)NHR, —SH, —SR, —S(O)H, —S(O)R, —SO₂R, —SO₂NH₂, —SO₂NHR, —SO₂NRR, —CF₃, —CHF₂, —CH₂F, —OCF₃, —OCHF₂, —CN, —C—CH, —C—CR, —CH═CHR, —CH═CRR, —CR═CHR, —CR═CRR, —CO₂H, —CO₂R, —CHO, —C(O)R, —C(O)NH₂, —C(O)NHR, —C(O)NRR, —CONHSO₂H, —CONHSO₂R, —CONRSO₂R, cyclic C₃-C₇ alkylamino, imidazolyl, C₁-C₆-alkylpiperazinyl, morpholinyl and thiomorpholinyl;
or R¹ and R² taken together, or R² and R³ taken together, or R³ and R⁴ taken together can form a saturated or a partially saturated or a fully unsaturated 5- or 6-membered ring of carbon atoms optionally including 1 to 3 heteroatoms selected from O, N and S, and the ring is optionally substituted independently with 1 to 4 substituents selected from R;
each R is independently selected from any of the groups defined in paragraphs (a) and (b) below:
(a) an optionally substituted C₁₋₆ alkyl group, an optionally substituted C₂₋₆ alkenyl group, an optionally substituted C₂₋₆ alkynyl group and an optionally substituted C₃₋₇ cyclic alkyl group; wherein the one or more optional substituents for each of said alkyl, alkenyl, alkynyl and cyclic alkyl groups are each independently selected from the following groups: halo, —OH, —OR⁵, —OC(O)R⁵, —OC(O)NH₂, —OC(O)NHR⁵, —OC(O)NR⁵R⁵, —OP(O)(OH)₂, —OP(O)(OR⁵)₂, —NO₂, —NH₂, —NHR⁵, —NR⁵R⁵, —N⁺(O⁻)R⁵R⁵, —NHC(O)H, —NHC(O)R⁵, —NR⁵C(O)R⁵, —NHC(O)NH₂, —NHC(O)NR⁵R⁵, —NR⁵C(O)NHR⁵, —SH, —SR⁵, —S(O)H, —S(O)R⁵, —SO₂R⁵, —SO₂NH₂, —SO₂NHR⁵, —SO₂NR⁵R⁵, —CF₃, —CHF₂, —CH₂F, —OCF₃, —OCHF₂, —CN, —CO₂H, —CO₂R⁵, —CHO, —C(O)R⁵, —C(O)NH₂, —C(O)NHR⁵, —C(O)NR⁵R⁵, —CONHSO₂H, —C(O)NHSO₂R⁵, —C(O)NR⁵SO₂R⁵, cyclic C₃-C₇ alkylamino, imidazolyl, piperazinyl, morpholinyl, thiomorpholinyl, piperidinyl, azepanyl, pyrrolidinyl and azetidinyl; wherein each of the groups imidazolyl, piperazinyl, morpholinyl, thiomorpholinyl, piperidinyl, azepanyl, pyrrolidinyl and azetidinyl are optionally substituted by one or more of the following groups: C₁₋₆ alkyl, C₂₋₆ alkenyl, C₂₋₆ alkynyl, C₃₋₇ cyclic alkyl, halo, —OH, —OR⁷, —OC(O)R⁷, —OC(O)NH₂—OC(O)NHR⁷, —OC(O)NR⁷R⁷, —OP(O)(OH)₂, —OP(O)(OR⁷)₂, —NO₂, —NH₂, —NHR⁷, —NR⁷R⁷, —N⁺(O⁻) R⁷R⁷, —NHC(O)H, —NHC(O)R⁷, —NR⁷C(O)R⁷, —NHC(O)NH₂, —NHC(O)NR⁷R⁷, —NR⁷C(O)NHR⁷, —SH, —SR⁷, —S(O)H, —S(O)R⁷, —SO₂R⁷, —SO₂NH₂, —SO₂NHR⁷, —SO₂NR⁷R⁷, —CF₃, —CHF₂, —CH₂F, —OCF₃, —OCHF₂, —CN, —CO₂H, —CO₂R⁷, —CHO, —C(O)R⁷, —C(O)NH₂, —C(O)NHR⁷, —C(O)NR⁷R⁷, —CONHSO₂H, —C(O)NHSO₂R⁷, —C(O)NR⁷SO₂R⁷, an optionally substituted aryl, and an optionally substituted heteroaryl group having up to 12 carbon atoms and having one or more heteroatoms in its ring system which are each independently selected from O, N and S; and wherein the one or more optional substituents for each of said aryl and heteroaryl groups are each independently selected from the following groups: C₁₋₆ alkyl, C₂₋₆ alkenyl, C₂₋₆ alkynyl or C₃₋₇ cyclic alkyl, halo, —OH, —OR⁸, —OC(O)R⁸, —OC(O)NH₂, —OC(O)NHR⁸, —OC(O)NR⁸R⁸, —OP(O)(OH)₂, —OP(O)(OR⁸)₂, —NO₂, —NH₂, —NHR⁸, —NR⁸R⁸, —N⁺(O)R⁸R⁸, —NHC(O)H, —NHC(O)R⁸, —NR⁸C(O)R⁸, —NHC(O)NH₂, —NHC(O)NR⁸R⁸, —NR⁸C(O)NHR⁸, —SH, —SR⁸, —S(O)H, —S(O)R⁸, —SO₂R⁸, —SO₂NH₂, —SO₂NHR⁸, —SO₂NR⁸R⁸, —CF₃, —CHF₂, —CH₂F, —OCF₃, —OCHF₂, —CN, —CO₂H, —CO₂R⁸, —CHO, —C(O)R⁸, —C(O)NH₂, —C(O)NHR⁸, —C(O)NR⁸R⁸, —CONHSO₂H, —C(O)NHSO₂R⁸, and —C(O)NR⁸SO₂R⁸; wherein each R⁵, R⁷ and R⁸ is independently selected from a C₁₋₆ alkyl group, a C₂₋₆ alkenyl group, a C₂₋₆ alkynyl group and a C₃₋₇ cyclic alkyl group; and
(b) an optionally substituted aryl, and an optionally substituted heteroaryl group having up to 12 carbon atoms and having one or more heteroatoms in its ring system which are each independently selected from O, N and S; and wherein the one or more optional substituents are each independently selected from the same optional substituents as those defined in (a) above for R;
R⁹ and R¹⁰ are each independently selected from any of the groups defined in paragraphs (a) and (b) below:
(a) H, an optionally substituted C₁₋₆ alkyl group, an optionally substituted C₂₋₆ alkenyl group, an optionally substituted C₂₋₆ alkynyl group, and an optionally substituted C₃₋₇ cyclic alkyl group; wherein the one or more optional substituents for each of said alkyl, alkenyl, alkynyl and cyclic alkyl are each independently selected from the following groups: halo, —OH, —OR¹¹, —OC(O)R¹¹, —OC(O)NH₂, —OC(O)NHR¹¹, —OC(O)NR¹¹R¹¹, —OP(O)(OH)₂, —OP(O)(OR¹¹)₂, —NO₂, —NH₂, —NHR¹¹, —NR¹¹R¹¹, —N⁺(O)R¹¹R¹¹, —NHC(O)H, —NHC(O)R¹¹, —NR¹¹C(O)R¹¹, —NHC(O)NH₂, —NHC(O)NR¹¹R¹¹, —NR¹¹C(O)NHR¹¹, —SH, —SR¹¹, —S(O)H, —S(O)R¹¹, —SO₂R¹¹, —SO₂NH₂, —SO₂NHR¹¹, —SO₂NR¹¹R¹¹, —CF₃, —CHF₂, —CH₂F, —OCF₃, —OCHF₂, —CN, —CO₂H, —CO₂R¹¹, —CHO, —C(O)R¹¹, —C(O)NH₂, —C(O)NHR¹¹, —C(O)NR¹¹R¹¹, —CONHSO₂H, —C(O)NHSO₂R¹¹, —C(O)NR¹¹SO₂R¹¹, cyclic C₃-C₇ alkylamino, imidazolyl, piperazinyl, morpholinyl, thiomorpholinyl, piperidinyl, azepanyl, pyrrolidinyl and azetidinyl; wherein each of the groups cyclic C₃-C₇ alkylamino, imidazolyl, piperazinyl, morpholinyl, thiomorpholinyl, piperidinyl, azepanyl, pyrrolidinyl and azetidinyl are optionally substituted by one or more of the following groups: C₁₋₆ alkyl, C₂₋₆ alkenyl, C₂₋₆ alkynyl, C₃₋₇ cyclic alkyl, halo, —OH, —OR¹³, —OC(O)R¹³, —OC(O)NH₂, —OC(O)NHR¹³, —OC(O)NR¹³R¹³, —OP(O)(OH)₂, —OP(O)(OR¹³)₂, —NO₂, —NH₂, —NHR¹³, —NR¹³R¹³—N⁺(O)R¹³R¹³, —NHC(O)H, —NHC(O)R¹³, —NR¹³C(O)R¹³, —NHC(O)NH₂, —NHC(O)NR¹³R¹³, —NR¹³C(O)NHR¹³, —SH, —SR¹³, —S(O)H, —S(O)R¹³, —SO₂R¹³, —SO₂NH₂, —SO₂NHR¹³, —SO₂NR¹³R¹³, —CF₃, —CHF₂, —CH₂F, —OCF₃, —OCHF₂, —CN, —CO₂H, —CO₂R¹³, —CHO, —C(O)R¹³, —C(O)NH₂, —C(O)NHR¹³, —C(O)NR¹³R¹³, —CONHSO₂H, —C(O)NHSO₂R¹³, and —C(O)NR¹³SO₂R¹³;
wherein each R¹¹ and R¹³ is independently selected from a C₁₋₆ alkyl group, a C₂₋₆ alkenyl group, a C₂₋₆ alkynyl group and a C₃₋₇ cyclic alkyl group; and
(b) an optionally substituted aryl, and an optionally substituted heteroaryl group having up to 12 carbon atoms and having one or more heteroatoms in its ring system which are each independently selected from O, N and S; and wherein the one or more optional substituents for each of said aryl and heteroaryl are each independently selected from the same optional substituents as those defined in (a) above for R⁹ and R¹⁰;
or
(c) R⁹ and R¹⁰ taken together can form a partially saturated or a fully unsaturated 5- or 6-membered ring of carbon atoms optionally including 1 to 3 heteroatoms selected from O, N and S, and the ring can be optionally substituted independently with 1 to 5 substituents selected from the same optional substituents as those defined in (a) above for R⁹ and R¹⁰;
and a pharmaceutically acceptable carrier.

28. The method, use or dual inhibitor of claim 27, wherein Z is N or N-oxide, such as N, W is CR¹, X is CR² and Y is CR³.

29. The method, use or dual inhibitor of claim 27, wherein X is N or N-oxide, such as N, W is CR¹, Y is CR³ and Z is CR⁴.

30. The method, use or dual inhibitor of claim 27, wherein X and Z are both N or N-oxide, such as N, W is CR¹ and Y is CR³.

31. The method, use or dual inhibitor of any one of claims 27 to 30, wherein R¹, R², R³ and R⁴, where present, are each independently selected from the group consisting of H, halogen, optionally substituted C₁-C₆ alkyl, —O—R wherein R is selected from optionally substituted C₁-C₆ alkyl and optionally substituted aryl (such as phenyl), —NHR wherein R is optionally substituted aryl, an optionally substituted aryl, and an optionally substituted heteroaryl group having up to 12 carbon atoms and having one or more heteroatoms in its ring system which are each independently selected from O, N and S.

32. The method, use or dual inhibitor of claim 31, wherein R¹, R², R³ and R⁴, where present, are each independently selected from the group consisting of H, halogen, —CF₃, —CHF₂, —OCF₃, —OCHF₂, C₁₋₆ alkyl, such as methyl, substituted aryl, substituted heteroaryl, —OR wherein R is optionally substituted aryl, and —NHR wherein R is optionally substituted aryl.

33. The method, use or dual inhibitor of claim 31, wherein one or two of R¹, R², R³ and R⁴, where present, is H, and the others of R¹, R², R³ and R⁴ that are not H are independently selected from the group consisting of halogen, —CF₃, —CHF₂, —OCF₃, —OCHF₂, C₁₋₆ alkyl, such as methyl, substituted aryl, substituted heteroaryl, —OR wherein R is optionally substituted aryl, and —NHR wherein R is optionally substituted aryl.

34. The method, use or dual inhibitor of any one of claims 31 to 33, wherein R³ is present and selected from the group consisting of halogen, —OR wherein R is optionally substituted aryl, and —NHR wherein R is optionally substituted aryl.

35. The method, use or dual inhibitor of claim 28, wherein Z is N or N-oxide, such as N, W is CR¹, X is CR² and Y is CR³, and R³ is selected from the group consisting of halogen, —O—R wherein R is optionally substituted aryl, and —NHR wherein R is optionally substituted aryl.

36. The method, use or dual inhibitor of claim 28, wherein Z is N or N-oxide, such as N, W is CR¹, X is CR² and Y is CR³, R¹ is H, and one or both of R² and R³ are other than H.

37. The method, use or dual inhibitor of claim 36, wherein each of R² and R³ that is other than H is independently selected from the group consisting of halogen, optionally substituted C₁-C₆ alkyl, —OR wherein R is selected from optionally substituted C₁-C₆ alkyl and optionally substituted aryl, —NHR wherein R is optionally substituted aryl; an optionally substituted aryl, such as substituted phenyl, and an optionally substituted heteroaryl group.

38. The method, use or dual inhibitor of claim 36, wherein each of R² and R³ that is other than H is each independently selected from the group consisting of halogen, —CF₃, —CHF₂, —OCF₃, —OCHF₂, C₁₋₆ alkyl such as methyl, substituted aryl, substituted heteroaryl, —OR wherein R is optionally substituted aryl, and —NHR wherein R is optionally substituted aryl.

39. The method, use or dual inhibitor of any one of claims 27 to 30, wherein R¹ and R² taken together, or R² and R³ taken together, or R³ and R⁴ taken together form a saturated or a partially saturated or a fully unsaturated 5- or 6-membered ring of carbon atoms optionally including 1 to 3 heteroatoms selected from O, N or S and the ring is optionally substituted with 1 to 4 substituents independently selected from R, and those of R¹, R², R³ and R⁴ that are not part of the ring, are independently selected from: H, halo, optionally substituted C₁-C₆ alkyl, O—R wherein R is optionally substituted C₁-C₆ alkyl, an optionally substituted aryl and an optionally substituted heteroaryl group having up to 12 carbon atoms and having one or more heteroatoms in its ring system which are each independently selected from O, N and S.

40. The method, use or dual inhibitor of any one of claims 27 to 39 wherein R⁹ and R¹⁰ are independently selected from the group consisting of H, an optionally substituted C₁₋₆ alkyl group, an optionally substituted aryl, such as substituted phenyl, and an optionally substituted heteroaryl group having up to 12 carbon atoms and having one or more heteroatoms in its ring system which are each independently selected from O, N and S.

41. The method, use or dual inhibitor of any one of claims 27 to 40, wherein R⁹ and R¹⁰ are both H.

42. The method, use or dual inhibitor of claim 28, wherein Z is N or N-oxide, such as N, W is CR¹, X is CR² and Y is CR³, and R⁹ and R¹⁰ are both H.

43. The method, use or dual inhibitor of claim 42, wherein R¹, R² and R³ are each independently selected from the group consisting of H, halogen, —CF₃, —CHF₂, —OCF₃, —OCHF₂, C₁₋₆ alkyl, such as methyl, substituted aryl, substituted heteroaryl, —OR wherein R is optionally substituted aryl, and —NHR wherein R is optionally substituted aryl.

44. The method, use or dual inhibitor of claim 42, wherein one or two of R¹, R² and R³ is H, and the others of R¹, R² and R³ that are not H are independently selected from the group consisting of halogen, —CF₃, —CHF₂, —OCF₃, —OCHF₂, C₁₋₆ alkyl, such as methyl, substituted aryl, substituted heteroaryl, —OR wherein R is optionally substituted aryl, and —NHR wherein R is optionally substituted aryl.

45. The method, use or dual inhibitor of claim 42, wherein R¹ is H, and one of both of R² and R³ is other than H, wherein each of R² and R³ that is not H is independently selected from the group consisting of halogen, —CF₃, —CH F₂, —OCF₃, —OCHF₂, C₁₋₆ alkyl, such as methyl, substituted aryl, substituted heteroaryl, —OR wherein R is optionally substituted aryl, and —NHR wherein R is optionally substituted aryl.

46. The method, use or dual inhibitor of claim 42, wherein R³ is selected from the group consisting of halogen, —OR wherein R is optionally substituted aryl, and —NHR wherein R is optionally substituted aryl.

47. The method, use or dual inhibitor of claim 42, wherein R¹ is H, and R² and R³ form a saturated or a partially saturated or a fully unsaturated 5- or 6-membered ring of carbon atoms optionally including 1 to 3 heteroatoms selected from O, N and S and the ring is optionally substituted with 1 to 4 substituents independently selected from R.

48. The method, use or dual inhibitor of any one of claims 27 to 30 wherein W is CR¹ and R¹ is selected from H, halo, —CHF₂, —CF₃ or methyl; and Z is N or N-oxide, such as N, X is CR² and Y is CR³; X is N or N-oxide, such as N, Y is CR³ and Z is CR⁴; or X and Z are both N or N-oxide, such as N, and Y is CR³.

49. The method, use or dual inhibitor of any one of claims 27 to 30 wherein W is CR¹ and R¹ is selected from H, halo, —CHF₂, —CF₃ or methyl; R⁹ and R¹⁰ are each H; and Z is N or N-oxide, such as N, X is CR² and Y is CR³; X is N or N-oxide, such as N, Y is CR³ and Z is CR⁴; or X and Z are both N or N-oxide, such as N, and Y is CR³.

50. The method, use or dual inhibitor of any one of claims 27 to 30 wherein W is CR¹ and R¹ is selected from H, halo, —CHF₂, —CF₃ or methyl; R⁹ and R¹⁰ are each H; Z is N or N-oxide, such as N, X is CR² and Y is CR³; and R² and R³ are each independently selected from: H, halogen, —CF₃, —CHF₂, —OCF₃, —OCHF₂, —NO₂, C₁₋₆ alkyl, such as methyl, optionally substituted aryl, optionally substituted heteroaryl, —OR wherein R is optionally substituted aryl, and —NHR wherein R is optionally substituted aryl; or R² and R³ taken together can form a saturated or a partially saturated or a fully unsaturated 5- or 6-membered ring of carbon atoms and the ring is optionally substituted with 1 to 4 substituents independently selected from R.

51. The method, use or dual inhibitor of any one of claims 27 to 30 wherein W is CR¹ and R¹ is selected from H, halo, —CHF₂, —CF₃ or methyl.

52. The method, use or dual inhibitor of any one of claims 27 to 30 and 48 to 51 wherein W is CR¹ and R¹ is H, F, —CHF₂, —CF₃ or methyl.

53. The method, use or dual inhibitor of any one of claims 27 to 30 and 48 or 52 wherein W is CR¹ and R¹ is H or F.

54. The method, use or dual inhibitor of any one of claims 27 to 30 and 48 to 52 wherein W is CR¹ and R¹ is H or methyl.

55. The method, use or dual inhibitor of any one of claims 27 to 30 and 48 to 54 wherein W is CR¹ and R¹ is H.

56. The method, use or dual inhibitor of any one of claims 27 to 30 and 51 to 55 wherein R⁹ and R¹⁰ are independently selected from H, an optionally substituted C₁₋₆ alkyl group, an optionally substituted aryl, such as substituted phenyl, and an optionally substituted heteroaryl group having up to 12 carbon atoms and having one or more heteroatoms in its ring system which are each independently selected from O, N and S.

57. The method, use or dual inhibitor of any one of claims 27 to 30 and 51 to 56 wherein R⁹ and R¹⁰ are each H.

58. The method, use or dual inhibitor of any one of claims 48 to 57, wherein Z is N or N-oxide, such as N, X is CR² and Y is CR³.

59. The method, use or dual inhibitor of any one of claims 48 to 57, wherein X is N or N-oxide, such as N, Y is CR³ and Z is CR⁴.

60. The method, use or dual inhibitor of any one of claims 48 to 57, wherein X and Z are both N or N-oxide, such as N, and Y is CR³.

61. The method, use or dual inhibitor of any one of claims 27 to 30 and 51 to 58 wherein R² and R³ are each independently selected from: H, halogen, —CF₃, —CHF₂, —OCF₃, —OCHF₂, —NO₂, C₁₋₆ alkyl, such as methyl, optionally substituted aryl, optionally substituted heteroaryl, —OR wherein R is optionally substituted aryl, and —NHR wherein R is optionally substituted aryl; or R² and R³ taken together can form a saturated or a partially saturated or a fully unsaturated 5- or 6-membered ring of carbon atoms and the ring is optionally substituted with 1 to 4 substituents independently selected from R.

62. The method, use or dual inhibitor of any one of claims 27 to 30, 50 to 58, and 61 wherein R² and R³ are each independently selected from: H, halogen, —CF₃, —CHF₂, —OCF₃, —OCHF₂, —NO₂, C₁₋₆ alkyl, such as methyl, optionally substituted aryl, optionally substituted heteroaryl, —OR wherein R is optionally substituted aryl, and —NHR wherein R is optionally substituted aryl; or R² and R³ taken together can form a saturated or a partially saturated or a fully unsaturated 6-membered ring of carbon atoms and the ring is optionally substituted with 1 to 4 substituents independently selected from R.

63. The method, use or dual inhibitor of any one of claims 27 to 30, 50 to 58, 61 and 62 wherein R² and R³ are each independently selected from: H, halogen, —CF₃, —CHF₂, —OCF₃, —OCHF₂, —NO₂, C₁₋₆ alkyl, such as methyl, optionally substituted aryl, optionally substituted heteroaryl, —OR wherein R is optionally substituted aryl, and —NHR wherein R is optionally substituted aryl; or R² and R³ taken together can form a saturated or a partially saturated or a fully unsaturated 6-membered ring of carbon atoms.

64. The method, use or dual inhibitor of any one of claims 27 to 30, 50 to 58 and 61 to 63 wherein R² and R³ are each independently selected from: H, halo, —CF₃, —CHF₂, —NO₂, and methyl.

65. The method, use or dual inhibitor of any one of claims 27 to 30, 50 to 58, and 61 to 64 wherein R² and R³ are each independently selected from: H, halo, —CF₃, and —NO₂.

66. The method, use or dual inhibitor of any one of claims 27 to 30, 50 to 57, and 60 to 65 wherein one of R² and R³ is H and the other is a group other than H.

67. The method, use or dual inhibitor of claim 27, wherein W is CR¹ and R¹ is H or F; R⁹ and R¹⁰ are each H; Z is N or N-oxide, such as N, X is CR² and Y is CR³; and R² and R³ are each independently selected from: H, halo, —CF₃, —CHF₂, —NO₂, and methyl.

68. The method, use or dual inhibitor of claim 27, wherein W is CR¹ and R¹ is H; R⁹ and R¹⁰ are each H; Z is N or N-oxide, such as N, X is CR² and Y is CR³; and R² and R³ are each independently selected from: H, halo, —CF₃, and —NO₂.

69. The method, use or dual inhibitor of claim 27, 48, 49, 51 to 57 and 59, wherein X is N or N-oxide, such as N, Y is CR³ and Z is CR⁴; and R³ and R⁴ are each independently selected from the group of substituents listed for R² and R³ in any one of claims 61 to 68.

70. The method, use or dual inhibitor of claim 27, wherein X and Z are both N or N-oxide, such as N, and Y is CR³; and R³ is selected from the group of substituents listed for R² and R³ in any one of claims 61 to 68.

71. The method, use or dual inhibitor of any one of claims 1 to 27 wherein the dual inhibitor of IDO1 and TDO is selected from the group consisting of:
5-Bromo-4,6-dimethylisoxazolo[5,4-b]pyridin-3-amine (1)
Isoxazolo[5,4-b]pyridin-3-amine (2)
5-Chloro-4,6-dimethylisoxazolo[5,4-b]pyridin-3-amine (3)
4,6-Dimethylisoxazolo[5,4-b]pyridin-3-amine (4)
4,5,6-Trimethylisoxazolo[5,4-b]pyridin-3-amine (5)
5-Bromoisoxazolo[5,4-b]pyridin-3-amine (6)
6-Methylisoxazolo[5,4-b]pyridin-3-amine (7)
5-Chloroisoxazolo[5,4-b]pyridin-3-amine (8)
Isoxazolo[5,4-b]quinolin-3-amine (9)
5,6,7,8-Tetrahydroisoxazolo[5,4-b]quinolin-3-amine (10)
6-Chloroisoxazolo[5,4-b]pyridin-3-amine (11)
Isoxazolo[5,4-d]pyrimidin-3-amine (12)
5-Fluoroisoxazolo[5,4-b]pyridin-3-amine (14)
6-Phenylisoxazolo[5,4-b]pyridin-3-amine (15)
5-Iodoisoxazolo[5,4-b]pyridin-3-amine (16)
Isoxazolo[4,5-c]pyridin-3-amine (17)
N⁶,N⁶-Dimethylisoxazolo[5,4-b]pyridine-3,6-diamine (18)
N⁴,N⁴-Dimethylisoxazolo[5,4-b]pyridine-3,4-diamine (19)
5-(3-Methoxyphenyl)isoxazolo[5,4-b]pyridin-3-amine (22)
5-(2-Methoxyphenyl)isoxazolo[5,4-b]pyridin-3-amine (23)
5-Phenylisoxazolo[5,4-b]pyridin-3-amine (24)
5-(Pyridin-3-yl)isoxazolo[5,4-b]pyridin-3-amine (26)
5-(Pyridin-4-yl)isoxazolo[5,4-b]pyridin-3-amine (27)
2-(3-Aminoisoxazolo[5,4-b]pyridin-5-yl)phenol (28)
4-(3-Aminoisoxazolo[5,4-b]pyridin-5-yl)phenol (29)
5-(4-Fluorophenyl)isoxazolo[5,4-b]pyridin-3-amine (30)
5-(3-Fluorophenyl)isoxazolo[5,4-b]pyridin-3-amine (31)
5-(2,4-difluorophenyl)isoxazolo[5,4-b]pyridin-3-amine (32)
5-(3,5-Difluoro-2-methoxyphenyl)isoxazolo[5,4-b]pyridin-3-amine (33)
5-(2,4-Dichlorophenyl)isoxazolo[5,4-b]pyridin-3-amine (34)
5-(2,3,4-Trichlorophenyl)isoxazolo[5,4-b]pyridin-3-amine (35)
5-(4-(Trifluoromethylphenyl)isoxazolo[5,4-b]pyridin-3-amine (36)
5-(3-Aminophenyl)isoxazolo[5,4-b]pyridin-3-amine (37)
Methyl 3-(3-aminoisoxazolo[5,4-b]pyridin-5-yl)benzoate (38)
5-(6-Fluoropyridin-3-yl)isoxazolo[5,4-b]pyridin-3-amine (39)
5-(2-Chloro-4-(trifluoromethyl)phenyl)isoxazolo[5,4-b]pyridin-3-amine (40)
6-Methoxyisoxazolo[5,4-b]pyridin-3-amine (41)
6-Chloro-4-methylisoxazolo[5,4-b]pyridin-3-amine (42)
Isoxazolo[5,4-b]pyridine-3,6-diamine (43)
5-Methylisoxazolo[5,4-b]pyridin-3-amine (44)
5,6-Dimethylisoxazolo[5,4-b]pyridin-3-amine (45)
6-Methyl-4-(trifluoromethyl)isoxazolo[5,4-b]pyridin-3-amine (46)
6-(Trifluoromethyl)isoxazolo[5,4-b]pyridin-3-amine (47)
6-Isopropylisoxazolo[5,4-b]pyridin-3-amine (48)
5-Nitroisoxazolo[5,4-b]pyridin-3-amine (49)
Ethyl 3-amino-6-(trifluoromethyl)isoxazolo[5,4-b]pyridine-5-carboxylate (50)
4-Methoxyisoxazolo[5,4-b]pyridin-3-amine (51)
5-(Difluoromethoxy)-4,6-dimethylisoxazolo[5,4-b]pyridin-3-amine (52)
Ethyl 3-amino-6-methylisoxazolo[5,4-b]pyridine-5-carboxylate (53)
Ethyl 3-amino-6-(difluoromethyl)isoxazolo[5,4-b]pyridine-5-carboxylate (54)
5-Fluoro-6-morpholinoisoxazolo[5,4-b]pyridin-3-amine (55)
6-(Furan-2-yl)isoxazolo[5,4-b]pyridin-3-amine (58)
6,7,8,9-Tetrahydro-5H-cyclohepta[b]isoxazolo[4,5-e]pyridin-3-amine (60)
6,6-Dimethyl-5,6,7,8-tetrahydroisoxazolo[5,4-b]quinolin-3-amine (61)
7,8-Dihydro-5H-isoxazolo[5,4-b]pyrano[3,4-e]pyridin-3-amine (62)
6-(Methylthio)isoxazolo[5,4-d]pyrimidin-3-amine (63)
6-Methylisoxazolo[5,4-d]pyrimidin-3-amine (64)
6-Chloro-5-fluoroisoxazolo[5,4-b]pyridin-3-amine (66)
5,6-Dichloroisoxazolo[5,4-b]pyridin-3-amine (67)
6-Chloro-4-(trifluoromethyl)isoxazolo[5,4-b]pyridin-3-amine (68)
5-(3-Methoxyprop-1-yn-1-yl)isoxazolo[5,4-b]pyridin-3-amine (69)
6-(4-Fluorophenyl)isoxazolo[5,4-b]pyridin-3-amine (71)
6-(2,4-Difluorophenyl)isoxazolo[5,4-b]pyridin-3-amine (73)
6-(2-Thienyl)isoxazolo[5,4-b]pyridin-3-amine (74)
6-(Methylthio)isoxazolo[5,4-b]pyridin-3-amine (79)
6-(Methylsulfonyl)isoxazolo[5,4-b]pyridin-3-amine (80)
Methyl 3-aminoisoxazolo[5,4-b]pyridine-6-carboxylate (82)
6-Phenoxyisoxazolo[5,4-b]pyridin-3-amine (83)
6-(2-Chlorophenoxy)isoxazolo[5,4-b]pyridin-3-amine (84)
6-(3-Chlorophenoxy)isoxazolo[5,4-b]pyridin-3-amine (85)
6-(4-Chlorophenoxy)isoxazolo[5,4-b]pyridin-3-amine (86)
6-(2-(Trifluoromethoxy)phenoxy)isoxazolo[5,4-b]pyridin-3-amine (87)
6-(3-(Trifluoromethoxy)phenoxy)isoxazolo[5,4-b]pyridin-3-amine (88)
6-(4-(Trifluoromethoxy)phenoxy)isoxazolo[5,4-b]pyridin-3-amine (89)
6-(2-Methoxyphenoxy)isoxazolo[5,4-b]pyridin-3-amine (90)
6-(3-Methoxyphenoxy)isoxazolo[5,4-b]pyridin-3-amine (91)
6-(4-Methoxyphenoxy)isoxazolo[5,4-b]pyridin-3-amine (92)
6-(3-(Trifluoromethyl)phenoxy)isoxazolo[5,4-b]pyridin-3-amine (93)
N⁶-Phenylisoxazolo[5,4-b]pyridine-3,6-diamine (94)
N⁶-(3-Methoxyphenyl)isoxazolo[5,4-b]pyridine-3,6-diamine (95) and
N⁶-(4-Methoxyphenyl)isoxazolo[5,4-b]pyridine-3,6-diamine (96),
and pharmaceutically acceptable salts thereof,
and pharmaceutically acceptable salts thereof.

72. The method, use or dual inhibitor of any one of claims 1 to 27 wherein the dual inhibitor of IDO1 and TDO is selected from the group consisting of:
6-Chloro-5-fluoroisoxazolo[5,4-b]pyridin-3-amine (66)
5-Bromoisoxazolo[5,4-b]pyridin-3-amine (6)
5-Chloroisoxazolo[5,4-b]pyridin-3-amine (8)
6-Chloroisoxazolo[5,4-b]pyridin-3-amine (11)
5-Iodoisoxazolo[5,4-b]pyridin-3-amine (16)
5-Nitroisoxazolo[5,4-b]pyridin-3-amine (49)
5,6-Dichloroisoxazolo[5,4-b]pyridin-3-amine (67)
5-Chloro-4,6-dimethylisoxazolo[5,4-b]pyridin-3-amine (3)
4,6-Dimethylisoxazolo[5,4-b]pyridin-3-amine (4)
4,5,6-Trimethylisoxazolo[5,4-b]pyridin-3-amine (5)
6-Methylisoxazolo[5,4-b]pyridin-3-amine (7)
5-Phenylisoxazolo[5,4-b]pyridin-3-amine (24)
5-Bromo-4,6-dimethylisoxazolo[5,4-b]pyridin-3-amine (1)
Isoxazolo[5,4-b]quinolin-3-amine (9)
5-(4-Fluorophenyl)isoxazolo[5,4-b]pyridin-3-amine (30)
5,6,7,8-Tetrahydroisoxazolo[5,4-b]quinolin-3-amine (10)
Isoxazolo[5,4-d]pyrimidin-3-amine (12)
5-Fluoroisoxazolo[5,4-b]pyridin-3-amine (14)
6-Phenylisoxazolo[5,4-b]pyridin-3-amine (15)
6-(2-Thienyl)isoxazolo[5,4-b]pyridin-3-amine (74)
6-Methoxyisoxazolo[5,4-b]pyridin-3-amine (41)
6-(Trifluoromethyl)isoxazolo[5,4-b]pyridin-3-amine (47)
6-Chloro-4-methylisoxazolo[5,4-b]pyridin-3-amine (42)
5,6-Dimethylisoxazolo[5,4-b]pyridin-3-amine (45)
5-Methylisoxazolo[5,4-b]pyridin-3-amine (44)
6-(2-Chlorophenoxy)isoxazolo[5,4-b]pyridin-3-amine (84)
6-(4-(Trifluoromethoxy)phenoxy)isoxazolo[5,4-b]pyridin-3-amine (89)
N⁶-(3-Methoxyphenyl)isoxazolo[5,4-b]pyridine-3,6-diamine (95),
and pharmaceutically acceptable salts thereof
wherein the compound is both a dual inhibitor of IDO1 and TDO.

73. The method, use or dual inhibitor of any one of claims 1 to 27 wherein the dual inhibitor of IDO1 and TDO is selected from:
6-Chloro-5-fluoroisoxazolo[5,4-b]pyridin-3-amine (66)
5-Bromoisoxazolo[5,4-b]pyridin-3-amine (6)
5-Chloroisoxazolo[5,4-b]pyridin-3-amine (8)
6-Chloroisoxazolo[5,4-b]pyridin-3-amine (11)
5-Iodoisoxazolo[5,4-b]pyridin-3-amine (16)
5-Nitroisoxazolo[5,4-b]pyridin-3-amine (49)
5,6-Dichloroisoxazolo[5,4-b]pyridin-3-amine (67),
and pharmaceutically acceptable salts thereof.

74. The method, use or dual inhibitor of any one of claims 1 to 27 wherein the dual inhibitor of IDO1 and TDO is 6-Chloro-5-fluoroisoxazolo[5,4-b]pyridin-3-amine (66).

75. The method, use or dual inhibitor of claim 8 or 9 wherein the dual inhibitor of IDO1 and TDO is 6-Chloro-5-fluoroisoxazolo[5,4-b]pyridin-3-amine (66), or a pharmaceutically acceptable salt thereof, and wherein the anticancer agent is a platinum-based chemotherapeutic agent.

76. The method, use or dual inhibitor of claim 75 wherein the anticancer agent is selected from carboplatin, cisplatin, lobaplatin, oxaliplatin, picoplatin, nedaplatin, phenanthriplatin and satraplatin.

77. The method, use or dual inhibitor of claim 76 wherein the anticancer agent is selected from cisplatin, carboplatin and/or oxaliplatin.

78. The method, use or dual inhibitor of any one of claims 75 to 77 wherein the refractory cancer is selected from: lung cancer, pancreatic cancer, breast cancer and ovarian cancer.

79. The method, use or dual inhibitor of any one of claims 75 to 78 wherein the refractory cancer is a solid tumor.

80. The method, use or dual inhibitor of any one of the preceding claims wherein the dual inhibitor of IDO1 and TDO has a cellular IDO1 IC₅₀ of less than 100 μM as determined by a Cell-based assay for IDO1 inhibition; and has a cellular TDO IC₅₀ of less than 100 μM as determined by a Cell-based assay for TDO inhibition.

81. The method, use or dual inhibitor of any one of the preceding claims wherein the dual inhibitor of IDO1 and TDO is formulated or provided as a pharmaceutical composition comprising the dual inhibitor of IDO1 and TDO and a pharmaceutically acceptable carrier.

82. The method, use or dual inhibitor of claim 81 wherein the dual inhibitor of IDO1 and TDO is formulated or provided as a pharmaceutical composition comprising the dual inhibitor of IDO1 and TDO and a pharmaceutically acceptable carrier, for oral administration.

83. The method, use or dual inhibitor of any one of the preceding claims wherein the dual inhibitor of IDO1 and TDO is administered orally.

84. The method, use or dual inhibitor of any one of the preceding claims for preventing the development of refractory cancer or a refractory cancer cell, comprising administering to the subject or the cell the dual inhibitor of IDO1 and TDO in combination with the anticancer agent and/or cancer therapy which the cancer or cancer cell is to be prevented from becoming refractory to.

85. The method, use or dual inhibitor of claim 84 wherein the subject has cancer at risk of becoming refractory to the anticancer agent and/or therapy, or wherein the cancer cell is a cancer cell at risk of becoming refractory to the anticancer agent and/or therapy.

86. The method, use or dual inhibitor of claim 85 further comprising treating the cancer or cancer cell at risk of becoming refractory to the anticancer agent and/or therapy.

87. The method, use or dual inhibitor of any one of claims 84 to 86, wherein the anticancer agent and/or cancer therapy and the dual inhibitor of IDO1 and TDO are administered simultaneously, sequentially, or separately and, optionally, are administered as a single formulation or as separate formulations.

88. The method, use or dual inhibitor of any one of claims 85 to 87 wherein the anticancer agent and/or cancer therapy is selected from the anticancer agent and/or cancer therapy recited in any one of claims 12 to 17.

89. The method, use or dual inhibitor of any one of the preceding claims further comprising a step of administering one or more additional agents selected from the group consisting of: anticancer agents, immune-modulating agents such as anticancer vaccines, modulators of immune checkpoint proteins, adoptive T cell immunotherapies (for example chimeric antigen receptor T cells (CART cells)), and radiotherapy, and wherein the additional agent is administered either before, during or after administration of the dual inhibitor of IDO1 and TDO.

90. The method, use or dual inhibitor of claim 89, wherein the one or more additional agents is administered simultaneously or sequentially with the dual inhibitor of IDO1 and TDO.

91. The method, use or dual inhibitor of claim 89 or 90 wherein the one or more additional agents is an anticancer agent selected from the anticancer agents recited in any one of claims 12 to 17.

92. The method, use or dual inhibitor of claim 89 or 90 wherein the one or more additional agent is an immune-modulating agent selected from: an inhibitor of CTLA4, an anti-PD-1 antibody or an anti-PD-L1 antibody.

93. The method, use or dual inhibitor of claim 92 wherein the one or more additional agent is selected from: Ipilimumab, Cemiplimab, Nivolumab, Pembrolizumab, Atezolizumab, Avelumab and Durvalumab.

94. A kit comprising the dual inhibitor of indoleamine-2,3-dioxygenase (IDO1) and tryptophan-2,3-dioxygenase (TDO) of any one of the preceding claims; and optionally one or more additional therapeutic agent; and instructions for using the dual inhibitor in the method of the any one of the preceding claims.
